SEQUENCE LISTING (387 sequences)

SEQ ID NO: 1 - protein, 124 aa, unidentified
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, unidentified [sequence omitted]

SEQ ID NO: 3 - protein, 8 aa, unidentified
GFTFRDYA

SEQ ID NO: 4 - protein, 8 aa, unidentified
ISGSGGNT

SEQ ID NO: 5 - protein, 18 aa, unidentified
AKDRLSITIRPRYYGLDV

SEQ ID NO: 6 - protein, 11 aa, unidentified
QSLLYSIGYNY

SEQ ID NO: 7 - skipped

SEQ ID NO: 8 - protein, 9 aa, unidentified
MQALQTPYT

SEQ ID NO: 9 - protein, 452 aa, unidentified
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 219 aa, unidentified
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 207 aa, unidentified
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 12 - protein, 109 aa, unidentified
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 13 - protein, 109 aa, unidentified
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 14 - protein, 109 aa, unidentified
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 15 - protein, 109 aa, unidentified
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 16 - protein, 109 aa, unidentified
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 17 - protein, 109 aa, unidentified
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 18 - protein, 109 aa, unidentified
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 19 - protein, 109 aa, unidentified
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 20 - protein, 109 aa, unidentified
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 21 - protein, 109 aa, unidentified
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 22 - protein, 109 aa, unidentified
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 23 - protein, 109 aa, unidentified
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 24 - protein, 109 aa, unidentified
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 25 - protein, 109 aa, unidentified
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 26 - protein, 109 aa, unidentified
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 27 - protein, 109 aa, unidentified
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 28 - protein, 109 aa, unidentified
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 29 - protein, 109 aa, unidentified
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 30 - protein, 109 aa, unidentified
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 31 - protein, 109 aa, unidentified
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 32 - protein, 115 aa, unidentified
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, unidentified
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, unidentified
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 35 - protein, 115 aa, unidentified
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 36 - protein, 115 aa, unidentified
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 37 - protein, 115 aa, unidentified
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, unidentified
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, unidentified
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 40 - protein, 115 aa, unidentified
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 41 - protein, 115 aa, unidentified
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, unidentified
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, unidentified
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, unidentified
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 45 - protein, 115 aa, unidentified
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 46 - protein, 115 aa, unidentified
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 47 - protein, 115 aa, unidentified
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, unidentified
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, unidentified
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, unidentified
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 51 - protein, 115 aa, unidentified
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 52 - protein, 115 aa, unidentified
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 53 - protein, 115 aa, unidentified
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 54 - protein, 115 aa, unidentified
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, unidentified
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, unidentified
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 57 - protein, 115 aa, unidentified
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 58 - protein, 115 aa, unidentified
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 59 - protein, 115 aa, unidentified
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, unidentified
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, unidentified
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, unidentified
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 63 - protein, 115 aa, unidentified
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 64 - protein, 115 aa, unidentified
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 65 - protein, 115 aa, unidentified
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 66 - protein, 115 aa, unidentified
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 67 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 68 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 69 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 70 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 71 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 72 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 73 - protein, 116 aa, unidentified; identical to SEQ ID NO: 69

SEQ ID NO: 74 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 75 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 76 - protein, 111 aa, unidentified; identical to SEQ ID NO: 74

SEQ ID NO: 77 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 78 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 79 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 80 - protein, 111 aa, unidentified; identical to SEQ ID NO: 74

SEQ ID NO: 81 - protein, 116 aa, unidentified; identical to SEQ ID NO: 79

SEQ ID NO: 82 - protein, 111 aa, unidentified; identical to SEQ ID NO: 78

SEQ ID NO: 83 - protein, 116 aa, unidentified; identical to SEQ ID NO: 69

SEQ ID NO: 84 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 85 - protein, 116 aa, unidentified; identical to SEQ ID NO: 75

SEQ ID NO: 86 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 89 - protein, 116 aa, unidentified; identical to SEQ ID NO: 87

SEQ ID NO: 90 - protein, 111 aa, unidentified; identical to SEQ ID NO: 84

SEQ ID NO: 91 - protein, 116 aa, unidentified; identical to SEQ ID NO: 75

SEQ ID NO: 92 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 93 - protein, 116 aa, unidentified; identical to SEQ ID NO: 69

SEQ ID NO: 94 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 95 - protein, 116 aa, unidentified; identical to SEQ ID NO: 69

SEQ ID NO: 96 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 97 - protein, 116 aa, unidentified; identical to SEQ ID NO: 69

SEQ ID NO: 98 - protein, 111 aa, unidentified; identical to SEQ ID NO: 78

SEQ ID NO: 99 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 100 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 101 - protein, 116 aa, unidentified; identical to SEQ ID NO: 99

SEQ ID NO: 102 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 103 - protein, 116 aa, unidentified; identical to SEQ ID NO: 99

SEQ ID NO: 104 - protein, 111 aa, unidentified; identical to SEQ ID NO: 78

SEQ ID NO: 105 - protein, 116 aa, unidentified; identical to SEQ ID NO: 77

SEQ ID NO: 106 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 107 - protein, 116 aa, unidentified
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 108 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 109 - protein, 116 aa, unidentified; identical to SEQ ID NO: 77

SEQ ID NO: 110 - protein, 111 aa, unidentified
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 111 - protein, 116 aa, unidentified
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 112 - protein, 111 aa, unidentified
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 113 - protein, 116 aa, unidentified
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 114 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 115 - protein, 116 aa, unidentified
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 116 - protein, 111 aa, unidentified
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 117 - protein, 116 aa, unidentified; identical to SEQ ID NO: 77

SEQ ID NO: 118 - protein, 111 aa, unidentified; identical to SEQ ID NO: 114

SEQ ID NO: 119 - protein, 116 aa, unidentified
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 120 - protein, 111 aa, unidentified
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 121 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 122 - protein, 111 aa, unidentified
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 123 - protein, 116 aa, unidentified
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 124 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 125 - protein, 116 aa, unidentified; identical to SEQ ID NO: 77

SEQ ID NO: 126 - protein, 111 aa, unidentified
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 127 - protein, 116 aa, unidentified; identical to SEQ ID NO: 77

SEQ ID NO: 128 - protein, 111 aa, unidentified
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 129 - protein, 116 aa, unidentified; identical to SEQ ID NO: 77

SEQ ID NO: 130 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 131 - protein, 116 aa, unidentified
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 132 - protein, 111 aa, unidentified
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 133 - protein, 116 aa, unidentified
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 134 - protein, 111 aa, unidentified; identical to SEQ ID NO: 114

SEQ ID NO: 135 - protein, 116 aa, unidentified; identical to SEQ ID NO: 77

SEQ ID NO: 136 - protein, 111 aa, unidentified; identical to SEQ ID NO: 124

SEQ ID NO: 137 - protein, 116 aa, unidentified; identical to SEQ ID NO: 107

SEQ ID NO: 138 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 139 - protein, 116 aa, unidentified
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 140 - protein, 111 aa, unidentified
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 141 - protein, 116 aa, unidentified; identical to SEQ ID NO: 107

SEQ ID NO: 142 - protein, 111 aa, unidentified; identical to SEQ ID NO: 114

SEQ ID NO: 143 - protein, 116 aa, unidentified; identical to SEQ ID NO: 113

SEQ ID NO: 144 - protein, 111 aa, unidentified
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 145 - protein, 116 aa, unidentified; identical to SEQ ID NO: 77

SEQ ID NO: 146 - protein, 111 aa, unidentified; identical to SEQ ID NO: 138

SEQ ID NO: 147 - protein, 116 aa, unidentified
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 148 - protein, 111 aa, unidentified
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 149 - protein, 116 aa, unidentified
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 150 - protein, 111 aa, unidentified
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 151 - protein, 116 aa, unidentified
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 152 - protein, 111 aa, unidentified
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 153 - protein, 116 aa, unidentified
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 154 - protein, 121 aa, unidentified
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 155 - protein, 127 aa, unidentified
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 156 - protein, 116 aa, unidentified
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 157 - protein, 116 aa, unidentified
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 158 - protein, 116 aa, unidentified
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 159 - protein, 116 aa, unidentified
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 160 - protein, 116 aa, unidentified
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 161 - protein, 116 aa, unidentified
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 162 - protein, 116 aa, unidentified
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 163 - protein, 116 aa, unidentified
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 164 - protein, 116 aa, unidentified
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 165 - protein, 116 aa, unidentified
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 166 - protein, 116 aa, unidentified
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 167 - protein, 116 aa, unidentified
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 168 - protein, 110 aa, unidentified
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 169 - protein, 110 aa, unidentified
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 170 - protein, 110 aa, unidentified
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 171 - protein, 110 aa, unidentified
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 172 - protein, 110 aa, unidentified
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 173 - protein, 110 aa, unidentified
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 174 - protein, 110 aa, unidentified
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 175 - protein, 110 aa, unidentified
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 176 - protein, 110 aa, unidentified
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 177 - protein, 110 aa, unidentified
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 178 - protein, 110 aa, unidentified; identical to SEQ ID NO: 177

SEQ ID NO: 179 - protein, 110 aa, unidentified
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 180 - protein, 110 aa, unidentified
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 181 - protein, 110 aa, unidentified; identical to SEQ ID NO: 177

SEQ ID NO: 182 - protein, 111 aa, unidentified
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 183 - protein, 111 aa, unidentified
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 184 - protein, 111 aa, unidentified
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 185 - protein, 111 aa, unidentified
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 186 - protein, 111 aa, unidentified
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 187 - protein, 111 aa, unidentified
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 188 - protein, 117 aa, unidentified
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 189 - protein, 128 aa, unidentified
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 190 - protein, 122 aa, unidentified
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 191 - protein, 128 aa, unidentified
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 192 - protein, 117 aa, unidentified
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 193 - protein, 124 aa, unidentified
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 194 - protein, 117 aa, unidentified
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 195 - protein, 116 aa, unidentified
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 196 - protein, 117 aa, unidentified
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 197 - protein, 122 aa, unidentified
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 198 - protein, 107 aa, unidentified
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 199 - protein, 107 aa, unidentified
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 200 - protein, 107 aa, unidentified
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 201 - protein, 107 aa, unidentified
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 202 - protein, 107 aa, unidentified
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 203 - protein, 107 aa, unidentified
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 204 - protein, 107 aa, unidentified
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 205 - protein, 107 aa, unidentified
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 206 - protein, 107 aa, unidentified
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 207 - protein, 107 aa, unidentified
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK

SEQ ID NO: 208 - protein, 120 aa, unidentified
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVKVGYRGGMDVWGQGTTVTVSS

SEQ ID NO: 209 - protein, 120 aa, unidentified
EVQLLESGGGLVQPGGSLRLSCAASGFTLSSYAMHWVRQAPGKGLEYVSGISPSGSSTYYANSVKGRFTISRDNPKNTLFLQMSSLRAEDTAVYYCVRSKVRYRGGMDVWGQGTTVTVSS

SEQ ID NO: 210 - protein, 120 aa, unidentified
EVQLLESGGGLVQPGGSLRLSCAASGFTLSSYAMHWVRQAPGKGLEYVSGISPSGVSTYYANSVKGRFTISRDNPKNTLFLQMSSLRAEDTAVYYCVRVKVKYRGGMDVWGQGTTVTVSS

SEQ ID NO: 211 - protein, 120 aa, unidentified
EVQLLESGGGLVQPGGSLRLSCAASGFTLSSYAMHWVRQAPGKGLEYVSGISPTSGSTYYANSVKGRFTISRDNPKNTLFLQMSSLRAEDTAVYYCVRVKVRYRGGMDVWGQGTTVTVSS

SEQ ID NO: 212 - protein, 120 aa, unidentified
EVQLLESGGGLVQPGGSLRLSCAASGFTLSSYAMHWVRQAPGKGLEYVSGISPTGTSTYYANSVKGRFTISRDNPKNTLFLQMSSLRAEDTAVYYCVRVKGAYRGGMDVWGQGTTVTVSS

SEQ ID NO: 213 - protein, 120 aa, unidentified
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKVAYRGGMDVWGQGTTVTVSS

SEQ ID NO: 214 - protein, 120 aa, unidentified
EVQLLESGGGLVQPGGSLRLSCAASGFTLSSYAMHWVRQAPGKGLEYVSGISPSSTSTYYANSVKGRFTISRDNPKNTLFLQMSSLRAEDTAVYYCVRVKVLYRGGMDVWGQGTTVTVSS

SEQ ID NO: 215 - protein, 120 aa, unidentified
EVQLLESGGGLVQPGGSLRLSCAASGFTLSSYAMHWVRQAPGKGLEYVSGISPSSASTYYANSVKGRFTISRDNPKNTLFLQMSSLRAEDTAVYYCVRVKSKYRGGMDVWGQGTTVTVSS

SEQ ID NO: 216 - protein, 120 aa, unidentified
EVQLLESGGGLVQPGGSLRLSCAASGFTLSSYAMHWVRQAPGKGLEYVSGISGNSASTYYANSVKGRFTISRDNPKNTLFLQMSSLRAEDTAVYYCVRVKLKYRGGMDVWGQGTTVTVSS

SEQ ID NO: 217 - protein, 120 aa, unidentified
EVQLLESGGGLVQPGGSLRLSCAASGFTLSSYAMHWVRQAPGKGLEYVSGISHSGTSTYYANSVKGRFTISRDNPKNTLFLQMSSLRAEDTAVYYCVRVRVLYRGGMDVWGQGTTVTVSS

SEQ ID NO: 218 - protein, 120 aa, unidentified; identical to SEQ ID NO: 210

SEQ ID NO: 219 - protein, 120 aa, unidentified
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVFVRYRGGMDVWGQGTTVTVSS

SEQ ID NO: 220 - protein, 120 aa, unidentified
EVQLLESGGGLVQPGGSLRLSCAASGFTLSSYAMHWVRQAPGKGLEYVSGISPTSASTYYANSVKGRFTISRDNPKNTLFLQMSSLRAEDTAVYYCVRVKGRYRGGMDVWGQGTTVTVSS

SEQ ID NO: 221 - protein, 120 aa, unidentified
EVQLLESGGGLVQPGGSLRLSCAASGFTLSSYAMHWVRQAPGKGLEYVSGISPTGGSTYYANSVKGRFTISRDNPKNTLFLQMSSLRAEDTAVYYCVRVKGRYRGGMDVWGQGTTVTVSS

SEQ ID NO: 222 - protein, 120 aa, unidentified
EVQLLESGGGLVQPGGSLRLSCAASGFTLSSYAMHWVRQAPGKGLEYVSGISHSGNSTYYANSVKGRFTISRDNPKNTLFLQMSSLRAEDTAVYYCVRVKRRYRGGMDVWGQGTTVTVSS

SEQ ID NO: 223 - protein, 120 aa, unidentified
EVQLLESGGGLVQPGGSLRLSCAASGFTLSSYAMHWVRQAPGKGLEYVSGISPSSNSTYYANSVKGRFTISRDNPKNTLFLQMSSLRAEDTAVYYCVRVKVRYRGGMDVWGQGTTVTVSS

SEQ ID NO: 224 - protein, 120 aa, unidentified
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKPAYRGGMDVWGQGTTVTVSS

SEQ ID NO: 225 - protein, 120 aa, unidentified
EVQLLESGGGLVQPGGSLRLSCAASGFTLSSYAMHWVRQAPGKGLEYVSGISYSSASTYYANSVKGRFTISRDNPKNTLFLQMSSLRAEDTAVYYCVRVKVRYRGGMDVWGQGTTVTVSS

SEQ ID NO: 226 - protein, 108 aa, unidentified
ETTLTQSPDTLPLSPGDRASLSCRASQSVSSAYLAWYQQKPGQAPRLLIYGTSRRATGVPGRFSGSGSGTDFTLTISRLEPEDFAVYYCQLYGSSSVTFGQGTKLEIK

SEQ ID NO: 227 - protein, 108 aa, unidentified
EIVLTQSPGTLSLSPGERATLSCRASQGISSAYLAWYQQKPGQAPRLLIYGTSRRATGIPDRFSGSGSGTDFTLTISRLEPEDFAVYYCQLYGATSVTFGQGTKLEIK

SEQ ID NO: 228 - protein, 108 aa, unidentified; identical to SEQ ID NO: 227

SEQ ID NO: 229 - protein, 108 aa, unidentified
EIVLTQSPGTLSLSPGERATLSCRASQSVSSAYLAWYQQKPGQAPRLLIYGTSRRATGIPDRFSGSGSGTDFTLTISRLEPEDFAVYYCQLYGASSVTFGQGTKLEIK

SEQ ID NO: 230 - protein, 108 aa, unidentified; identical to SEQ ID NO: 229

SEQ ID NO: 231 - protein, 108 aa, unidentified; identical to SEQ ID NO: 227

SEQ ID NO: 232 - protein, 108 aa, unidentified; identical to SEQ ID NO: 229

SEQ ID NO: 233 - protein, 108 aa, unidentified
EIVLTQSPGTLSLSPGERATLSCRASQSISTAYLAWYQQKPGQAPRLLIYGTSRRATGIPDRFSGSGSGTDFTLTISRLEPEDFAVYYCQLYGASSVTFGQGTKLEIK

SEQ ID NO: 234 - protein, 108 aa, unidentified
EIVLTQSPGTLSLSPGERATLSCRASQDISSAYLAWYQQKPGQAPRLLIYGTSRRATGIPDRFSGSGSGTDFTLTISRLEPEDFAVYYCQLYGATSVTFGQGTKLEIK

SEQ ID NO: 235 - protein, 108 aa, unidentified
EIVLTQSPGTLSLSPGERATLSCRASQDVSSAYLAWYQQKPGQAPRLLIYGTSRRATGIPDRFSGSGSGTDFTLTISRLEPEDFAVYYCQLYGATSVTFGQGTKLEIK

SEQ ID NO: 236 - protein, 108 aa, unidentified
EIVLTQSPGTLSLSPGERATLSCRASQNISTAYLAWYQQKPGQAPRLLIYGTSRRATGIPDRFSGSGSGTDFTLTISRLEPEDFAVYYCQLYGATSVTFGQGTKLEIK

SEQ ID NO: 237 - protein, 108 aa, unidentified
EIVLTQSPGTLSLSPGERATLSCRASQDASNAYLAWYQQKPGQAPRLLIYGTSRRATGIPDRFSGSGSGTDFTLTISRLEPEDFAVYYCQLYGSSSVTFGQGTKLEIK

SEQ ID NO: 238 - protein, 108 aa, unidentified
EIVLTQSPGTLSLSPGERATLSCRASQGVSSAYLAWYQQKPGQAPRLLIYGTSRRATGIPDRFSGSGSGTDFTLTISRLEPEDFAVYYCQLYGRSSVTFGQGTKLEIK

SEQ ID NO: 239 - protein, 108 aa, unidentified
EIVLTQSPGTLSLSPGERATLSCRASQNISTAYLAWYQQKPGQAPRLLIYGTSRRATGIPDRFSGSGSGTDFTLTISRLEPEDFAVYYCQLYGTSSVTFGQGTKLEIK

SEQ ID NO: 240 - protein, 108 aa, unidentified
EIVLTQSPGTLSLSPGERATLSCRASQSVSTAYLAWYQQKPGQAPRLLIYGTSRRATGIPDRFSGSGSGTDFTLTISRLEPEDFAVYYCQLYGATSVTFGQGTKLEIK

SEQ ID NO: 241 - protein, 108 aa, unidentified; identical to SEQ ID NO: 234

SEQ ID NO: 242 - protein, 108 aa, unidentified
EIVLTQSPGTLSLSPGERATLSCRASQGVSTAYLAWYQQKPGQAPRLLIYGTSRRATGIPDRFSGSGSGTDFTLTISRLEPEDFAVYYCQLYGATSVTFGQGTKLEIK

SEQ ID NO: 243 - protein, 108 aa, unidentified
EIVLPQSPGTLSLSPGERATLSCRASQGVSSAYLAWYQQKPGQAPRLLIYGTSRRATGIPDRFSGSGSGTDFTLTISRLEPEDFAVYYCQLYGSTSVTFGQGTKLEIK

SEQ ID NO: 244 - protein, 118 aa, unidentified
EVQLVESGGGLVQPKGSLKLSCAASGFTFNTYGMHWVRQAPGKGLEWVAHIRSKSSNYATYYADSVKDRFTISRDDSQSMLYLQMNNLKTEDTAMYYCVRWFRAMDYWGQGTSVTVSS

SEQ ID NO: 245 - protein, 120 aa, unidentified
EVQLIESGGGLVQPKGSLKLSCAASGFTFNMYAMDWVRQAPGKGLEWVARIRSKGSNFETNYADSVKDRFTISRDDSQSMVYLQMINLKTEDTAMYYCVRHRGGAWFAYWGQGTLVSVSA

SEQ ID NO: 246 - protein, 121 aa, unidentified
QVQLVETGGGLVRPGNSLKLSCVTSGFTFSNYRMHWLRQPPGKRLEWIAVITVKSNNYGANYAESVKGRFAISRDDSKSSVYLEMNRLREEDTATYFCSRERAYGNPFDYWGQGTTLTVSS

SEQ ID NO: 247 - protein, 118 aa, unidentified
EVQLVESGGGLVQPKGSLKLSCAASGFTFNMYAMNWVRQAPGQGLEWVARIRSKSNNYATYYADSVKDRFIISRDDSESMVYLQMSNLRAADTAMYYCVRHLRAMDYWGQGTSVTVSS

SEQ ID NO: 248 - protein, 118 aa, unidentified
EVQLVESGGGLVQPKGSLKLSCAASGFSFNMYAMNWVRQAPGKGLEWVARIRTKSNHYSTYYADSVKDRFTISRDDSASMFYLQMNNLKTEDTAMYFCVRHLRAMDYWGQGTSVTVSS

SEQ ID NO: 249 - protein, 120 aa, unidentified
EVQLIESGGGLVQPKGSLKLSCAASGFTFNMYAMDWVRQAPGKGLEWVARIRSKGSNFETNYADSVKDRFTISRDDSQSMVYLQMNNLKTEDTAMYYCVRHRGGAWFAYWGQGTLVTVSA

SEQ ID NO: 250 - protein, 118 aa, unidentified
EVQLVESGGGLVRPKGSLKLSCAASGFSFNTYAMNWVRQAPGKGLEWIVWIRSKSHNYATYYADSVKDRFTISRDDSESMLYLQMNNLKTEDTAMYYCVRHLRAMDYWGQGTSVTVSS

SEQ ID NO: 251 - protein, 118 aa, unidentified
EVRLVESGGGLVQPKGSLKLSCEASGFSFNMYAMNWVRQAPGKGLEWITHIRSKSNNYATYYADSVKDRFIISRDDSESMVYLQMNNLKTEDTAMYYCVRLLRALDYWGQGTSVTVSS

SEQ ID NO: 252 - protein, 118 aa, unidentified
EVQLVESGGGLVQPKGSLKLSCAASGFTFNMYGMHWVRQAPGKGLEWVAHIRSKSSNYATYYADSVKDRLTISRDDSQSMLYLQMNNLKTEDTAMYYCVRWFRAMDYWGQGTSVTVSS

SEQ ID NO: 253 - protein, 118 aa, unidentified
EVQLVESGGGLVKPGGSLRLSCAASGFTFSMYGMHWVRQAPGKGLEWVGHIRSKSSNYATYYADSVKDRFTISRDDSKNTLYLQMNSLKTEDTAVYYCTTWFRAMDYWGQGTLVTVSS

SEQ ID NO: 254 - protein, 118 aa, unidentified
EVQLVESGGGLVQPGGSLRLSCAASGFTFSMYGMHWVRQAPGKGLEWVSHIRSKSSNYATYYADSVKDRFTISRDNAKNSLYLQMNSLRAEDTAVYYCARWFRAMDYWGQGTLVTVSS

SEQ ID NO: 255 - protein, 118 aa, unidentified
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTLVTVSS

SEQ ID NO: 256 - protein, 118 aa, unidentified
EVQLVESGGGLVQPGGSLRLSCAASGFTFSMYGMHWVRQAPGKGLEWVGHIRSKSSNYATYYADSVKDRFTISRDDSKNSLYLQMNSLKTEDTAVYYCARWFRAMDYWGQGTLVTVSS

SEQ ID NO: 257 - protein, 118 aa, unidentified; identical to SEQ ID NO: 254

SEQ ID NO: 258 - protein, 118 aa, unidentified
EVQLVESGGGLEQPGGSLRLSCAGSGFTFRMYGMHWVRQAPGKGLEWVSHIRSKSSNYATYYADSVKDRFTISRDNSKNTLYLQMNSLRAEDTAVYYCAKWFRAMDYWGQGTTVTVSS

SEQ ID NO: 259 - protein, 118 aa, unidentified
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTTVTVSS

SEQ ID NO: 260 - protein, 107 aa, unidentified
DIVMTQSHKFMSTSVGDRVSITCKASQDVSTAVAWYQEKPGQSPKLLIYWASTRHTGVPDRFTGSGSGTDYTLTISSVQAEDLALYYCQQHYSTPLTFGAGTKLELK

SEQ ID NO: 261 - protein, 111 aa, unidentified
DIVVTQSPASLAVSLGQRATISCRASKSVSTSGYSYMHWYQQKPGQPPKLLIYLASNLESGVPARFSGSGSGTDFTLNIHPVEEEDVAIYYCQHSRELPLTFGAGTKLELK

SEQ ID NO: 262 - protein, 107 aa, unidentified
DIQMTQSPSSLSASLGERVSLTCRASQEISGYLSWLQQKPDGTIKRLIYAASTLDSGVPKRFSGSRSGSDYSLTISSLESEDFADYYCLQYGSYPYTFGGGTKLEIK

SEQ ID NO: 263 - protein, 111 aa, unidentified
DIVLTQSPASLTVSLGQRATISCRASKSVSTSGYSYMHWYQQKPGQPPKLLIYLASNLESGVPARFSGSGSGTDFTLNIHPVEEEDAATYYCQHSRELPITFGSGTKLEIK

SEQ ID NO: 264 - protein, 111 aa, unidentified
DIVLTQSPASLVVSLGQRATISCRASQSVSTSGYSYMHWYQQKPGQPPKLLIYLASNVQSGVPARFSGSGSGTDFTLNIHPVEEEDVATYYCHHNRDLPFTFGSGTKLEIK

SEQ ID NO: 265 - protein, 111 aa, unidentified; identical to SEQ ID NO: 261

SEQ ID NO: 266 - protein, 111 aa, unidentified
DIVLTQSPASLAVSLGQRATISCRASKSVSASGYSYMHWYQQKPGQPPKLLIYLASNLQSGVPARFSGSGSGTDFTLNIHPVEEEDAATYYCQHSRELPPTFGGGTKLEIK

SEQ ID NO: 267 - protein, 111 aa, unidentified
DIVLTQSPASLAVFLGQRATISCRASKSVSTSGYSYMHWYQQKAGQPPKLLIYLASNLESGVPARFSGSGSGTDFTLNIHPVEEEDAATYYCHHSRELPITFGSGTKLEMK

SEQ ID NO: 268 - protein, 107 aa, unidentified; identical to SEQ ID NO: 260

SEQ ID NO: 269 - protein, 107 aa, unidentified
EIVLTQSPGTLSLSPGERATLSCKASQDVSTAVAWYQQKPGQAPRLLIYWASTRHTGIPDRFSGSGSGTDFTLTISRLEPEDFAVYYCQQHYSTPLTFGQGTKVEIK

SEQ ID NO: 270 - protein, 107 aa, unidentified
DIVMTQSPLSLPVTPGEPASISCKASQDVSTAVAWYLQKSGQSPQLLIYWASTRHTGVPDRFSGSGSGTDFTLKISRVEAEDVGFYYCQQHYSTPLTFGQGTKLEIK

SEQ ID NO: 271 - protein, 107 aa, unidentified
DIVMTQSPDSLAVSLGERATINCKASQDVSTAVAWYQQKPGQPPKLLIYWASTRHTGVPDRFSGSGSGTDFTLTISSLQAEDVAVYYCQQHYSTPLTFGGGTKVEIK

SEQ ID NO: 272 - protein, 107 aa, unidentified
DIQMTQSPSSLSASVGDRVTITCKASQDVSTAVAWYQQKPGKVPKLLIYWASTRHTGVPSRFSGSGSGTDFTLTISSLQPEDVATYYCQQHYSTPLTFGGGTKVEIK

SEQ ID NO: 273 - protein, 107 aa, unidentified
DIQLTQSPSFLSASVGDRVTITCKASQDVSTAVAWYQQKPGKAPKLLIYWASTRHTGVPSRFSGSGSGTEFTLTISSLQPEDFATYYCQQHYSTPLTFGGGTKVEIK

SEQ ID NO: 274 - protein, 107 aa, unidentified
DIQMTQSPSSLSASVGDRVTITCKASQDVSTAVAWYQQKPGKAPKLLLYWASTRHTGVPSRFSGSGSGTDYTLTISSLQPEDFATYYCQQHYSTPLTFGGGTKVEIK

SEQ ID NO: 275 - protein, 107 aa, unidentified
AIRMTQSPFSLSASVGDRVTITCKASQDVSTAVAWYQQKPAKAPKLFIYWASTRHTGVPSRFSGSGSGTDYTLTISSLQPEDFATYYCQQHYSTPLTFGGGTKVEIK

SEQ ID NO: 276 - protein, 118 aa, unidentified
QVQLQQSGTELVKPGASVKMSCKASVNTFTGYNMHWIKQTPGQGLEWIGGLHPGNGDSSYNQKFKGRATLTDKSSNTAYMQLSSLTSEDSAVYYCALTTAGRAWFPYWGQGTLVTVSA

SEQ ID NO: 277 - protein, 107 aa, unidentified
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPPTFGGGTKLEIK

SEQ ID NO: 278 - protein, 119 aa, unidentified
QVQLQQSGTELVKPGASVKMSCKASVNTFTGYNMHWIKQTPGQGLEWIGGLHPGNGDSSYNQKFKGRATLTADKSSNTAYMQLSSLTSEDSAVYYCALTTAGRAWFAYWGQGTLVTVSA

SEQ ID NO: 279 - protein, 106 aa, unidentified
DIQMTQTTSSLSASLGDRVTISCRASQDISNYLNWYQQKPDGTVKLLIYYTSRLHSGVPSRFSGSGSGTDYSLTISNLEQEDIATYFCQQGNTLHTFGGGTKLEIK

SEQ ID NO: 280 - protein, 119 aa, unidentified
QVQLQQSGAELVKPGASVKMSCKASVNTFAGYNMHWVKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 281 - protein, 107 aa, unidentified
DIQMIQSTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 282 - protein, 119 aa, unidentified
QVQLQQSGAELVKPGASVKMSCKASVNTFTGYNMHWVKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 283 - protein, 107 aa, unidentified
DIQMIQSTSSLSASLGDRVTISCRASQDISNYLNWYQQKPDGTVKLLIYYTSRLHSGVPSRFSGSGSGTDYSLTISNLEQEDIATYFCQQGNTLPLTFGAGTKLELK

SEQ ID NO: 284 - protein, 119 aa, unidentified
QVQLQQSGAELVKPGASVKMSCKASVNTFTGYNMHWVKQTPGQGLEWIGGLHPGNGDTSYNQKFKGKATLTADKSSNTAYMQLSSLTSEDSAVYYCALTTAGRAWFPYWGQGTLVTVSA

SEQ ID NO: 285 - protein, 107 aa, unidentified
DIQMIQSTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLELK

SEQ ID NO: 286 - protein, 119 aa, unidentified
QVQLQQSGAELVKPGASVKMSCKASVNTFTGYNMHWIKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 287 - protein, 107 aa, unidentified; identical to SEQ ID NO: 285

SEQ ID NO: 288 - protein, 119 aa, unidentified
EVQLQQSGAELVKPGASVKMSCKASVNIFTGYNMHWVKQTPGQGLEWIGGLHPGNGDTSYNQKFKDKATLTADKSSNTAYMQLSSLTSEDSAVYYCALTTAGRAWFAYWGQGTLVTVSA

SEQ ID NO: 289 - protein, 107 aa, unidentified
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLELK

SEQ ID NO: 290 - protein, 119 aa, unidentified
EVKLVESGGGLVKPGGSLKLSCAASGFTFSRYAMSWVRQTPEKRLEWVATISSGGSYTNYADSVKGRFTISRDNVKNTLYLQMNSLRAEDTAVYYCARATARATEFAYWGQGTLVTVSS

SEQ ID NO: 291 - protein, 106 aa, unidentified
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPPTFGGGTKLEL

SEQ ID NO: 292 - protein, 119 aa, unidentified; identical to SEQ ID NO: 288

SEQ ID NO: 293 - protein, 107 aa, unidentified
DIQMTQSTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIK

SEQ ID NO: 294 - protein, 119 aa, unidentified
EVQLKESGAELVKPGASVKMSCKASVNIFTGYNMHWVKQTPGQGLEWIGGLHPGNGDTSYNQKFKDKATLTADRSSNTAYMQLSSLTSEDSAVYYCALTTAGRAWFAYWGQGTLVTVSA

SEQ ID NO: 295 - protein, 107 aa, unidentified; identical to SEQ ID NO: 293

SEQ ID NO: 296 - protein, 119 aa, unidentified
QVQLKQSGAELVKPGASVKMSCTASVNIFTGYNMHWIKQTPGQGLEWIGGLHPGNGDTSYNQKFKDKATLTADRSSNTAYMQLSSLTSEDSAVYYCALTTAGRAWFAYWGQGTLVTVSA

SEQ ID NO: 297 - protein, 107 aa, unidentified
DIQMNQSTSSLSASLGDRVTISCRASQDISNYLNWYQQKPDGTIKLLIYYTSRLYSGVPSRFSGSGSGTDYSLTISNLEEEDIATYFCQQGNTIPYTFGGGTKLEIK

SEQ ID NO: 298 - protein, 119 aa, unidentified
EVQLQQSGAELVKPGASVKMSCKASVNIFTGYNMHWVKQTPGQGLEWIGGLHPGNGDTSYNQKFKDKATLTADRSSNTAYMQLSSLTSEDSAVYYCALTTAGRAWFAYWGQGTLVTVSA

SEQ ID NO: 299 - protein, 107 aa, unidentified
DIQMTQSTSSLSASLGDRVTISCRASQDISNYLNWYQQKPDGTIKLLIYYTSRLHSGVPSRFSGSGSGTDYSLTISNLEEEDIATYFCQQGNTLPWTFGGGTKLEIK

SEQ ID NO: 300 - protein, 119 aa, unidentified
EVQLQQSGAELVKPGASVKMSCKASVNIFTSYNMHWVKQTPGQGLEWIGGLHPGNGDTSYNQKFKGKATLTADKSSSTAYMQVSMLTSEDSAVYYCVLTTAGRAWFAYWGQGTLVTVSA

SEQ ID NO: 301 - protein, 107 aa, unidentified; identical to SEQ ID NO: 299

SEQ ID NO: 302 - protein, 119 aa, unidentified
QVQLQESGAELVKPGASVKMSCKASVNTFTGYNMHWVKQTPGQGLEWIGGLHPGNGDTSYNQKFKGKATLTADRSSNTAYMQLSSLTSEDSAVYYCALTTAGRAWFAYWGQGTLVTVSA

SEQ ID NO: 303 - protein, 107 aa, unidentified
DIQMTQTTSSLSASLGDRVTISCRASQDISNYLNWYQQKPDGTVKLLIYYTSRLHSGVPSRFSGSGSGTDYSLTISNLEEEDIATYFCQQGNTLPWTFGGGTKLEIK

SEQ ID NO: 304 - protein, 119 aa, unidentified
QVQLKQSGAELVKPGASVKMSCKASVNTFTGYNMHWVKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 305 - protein, 107 aa, unidentified
DIQMTQSTSSLSASLGDRVTISCRASQDISNYLNWYQQKPDGTVKLLIYYTSRLHSGVPSRFSGSGSGTDYSLTISNLEEEDIATYFCQQGNTLPWTFGGGTKLEIK

SEQ ID NO: 306 - protein, 119 aa, unidentified
QVQLKQSGAELVKPGASVKMSCKASVNTFTGYNMHWIKQTPGQGLEWIGGLHPGNGDTSYNQKFKGKATLTADKSSNTAYMQLSSLTSEDSAVYYCALTTAGRAWFAYWGQGTLVTVSA

SEQ ID NO: 307 - protein, 107 aa, unidentified
DIQMNQSTSSLSASLGDRVTISCRASQDISNYLNWYQQKPDGTIKLLIYYTSRLYSGVPSRFSGSGSGTDYSLTISNLEEEDIATYFCQQGNTLPYTFGGGTKLEIK

SEQ ID NO: 308 - protein, 119 aa, unidentified
QVQLQQSGAELVKPGASVKMSCKASVNTITGYNMHWVKQTPGQGLEWIGGLHPGNGDTSYNQKFKDKATLTADKSSNTAYMQLSSLTSEDSAVYYCALTTAGRAWFAYWGQGTLVTVSA

SEQ ID NO: 309 - protein, 108 aa, unidentified
DIKMTQSTSSLSASLGDRVTTSCRASQDISNYLNWYQQKPDGTVKLLIYYTSRLHSGVPSRFSGSGSGTDYSLTISNLEEEDIATYFCQQGNTLPPWTFGGGTKLEIK

SEQ ID NO: 310 - protein, 119 aa, unidentified
EVQLVESGGGLVKPGGSLRLSCAASGFTFSRYAMSWVRQAPGKGLEWVSTISSGGSYTNYADSVKGRFTISRDNVKNTLYLQMNSLRAEDTAVYYCARATARATEFAYWGQGTLVTVSS

SEQ ID NO: 311 - protein, 119 aa, unidentified
EVQLLESGGGLVQPGGSLRLSCAASGFTFSRYAMSWVRQAPGKGLEWVSTISSGGSYTNYADSVKGRFTISRDNVKNTLYLQMNSLRAEDTAVYYCARATARATEFAYWGQGTLVTVSS

SEQ ID NO: 312 - protein, 119 aa, unidentified
QVQLVQSGAELKKPGASVKVSCKASGNTFTGYNMHWIQQSPGQGLEWMGGLHPGNGDSSYNQKFQGRVTLTADKSSNTAYMELSSLRSEDSAVYYCALTTAGRAWFPYWGQGTLVTVSS

SEQ ID NO: 313 - protein, 119 aa, unidentified
QVQLVQSGAEVKKPGASVKMSCKASVYTFTGYNMHWIQQSPGQGLEWMGGLHPGNGDSSYNQKFQGRATLTADKSSNTAYMELSSLRSEDSAVYYCALTTAGRAWFPYWGQGTLVTVSS

SEQ ID NO: 314 - protein, 119 aa, unidentified
QVQLVQSGAEVKKPGASVKVSCKASGNIFTGYNMHWVRQAPGQGLEWMGGLHPGNGDSSYNQKFQGRVTLTADKSSNTAYMELSSLRSEDSAVYYCALTTAGRAWFAYWGQGTLVTVSS

SEQ ID NO: 315 - protein, 119 aa, unidentified
QVQLVQSGAEVKKPGASVKMSCKASVYTFTGYNMHWVRQAPGQGLEWIGGLHPGNGDSSYNQKFQGRATLTADKSSNTAYMELSSLRSEDSAVYYCALTTAGRAWFAYWGQGTLVTVSS

SEQ ID NO: 316 - protein, 119 aa, unidentified
EVQLVQSGAEVKKPGATVKISCKASGNIFTSYNMHWVRQAPGQGLEWMGGLHPGNGDTSYNQKFQGRVTLTADKSSSTAYMELSSLRSEDTAVYYCVLTTAGRAWFAYWGQGTLVTVSS

SEQ ID NO: 317 - protein, 119 aa, unidentified
EVQLVQSGAEVKKPGATVKMSCKASVYTFTSYNMHWVRQAPGQGLEWIGGLHPGNGDTSYNQKFKGRATLTADKSSSTAYMELSSLRSEDTAVYYCVLTTAGRAWFAYWGQGTLVTVSS

SEQ ID NO: 318 - protein, 107 aa, unidentified
DIQMTQSPSSLSASVGDRVTITCQASQDISNYLNWYQQKPGKAPKLLIYYTSRLHSGVPSRFSGSGSGTDYTFTISSLQPEDIATYFCQQGNTLPWTFGGGTKVEIK

SEQ ID NO: 319 - protein, 107 aa, unidentified
DIQMTQSPSSLSASVGDRVTITCRASQDISNYLNWYQQKPGKAPKLLIYYTSRLHSGVPSRFSGSGSGTDYTLTISSLQPEDFATYFCQQGNTLPWTFGGGTKVEIK

SEQ ID NO: 320 - protein, 107 aa, unidentified
DIQMTQSPSSLSASVGDRVTITCQASQDISNYLNWYQQKPGKAPKLLIYYTSRLHSGVPSRFSGSGSGTDYTLTISSLQPEDFATYFCQQGNTLPLTFGGGTKVEIK

SEQ ID NO: 321 - protein, 107 aa, unidentified
DIQMTQSPSSLSASVGDRVTITCQASQDISNYLNWYQQKPGKAPKLLIYYTSRLHSGVPSRFSGSGSGTDYTLTISSLQPEDFATYFCQQGNTLPWTFGGGTKVEIK

SEQ ID NO: 322 - protein, 116 aa, unidentified
EVQLQQSGPELVKPGASVKISCKTSGYTFTEYTIHWVKQNHGKSLEWIGGINPNNGGTVYNQNFKGKATLTVDKSSSTAYMELRSLTSEDSAVYYCARVRRGMDYWGQGTSVTVSS

SEQ ID NO: 323 - protein, 107 aa, unidentified
DIVMTQSHKFMSTSVGDRVSITCKASQDVTTAVAWYQQKPGQSPKLLIYSASYRYTGVPDRFTGSGSGTDFTFTISSVQAEDLAVYYCQQHYSAPWTFGGGTNLEIK

SEQ ID NO: 324 - protein, 116 aa, unidentified
QVQLQQSGAEVVKPGASVKISCKTSGYTFTEYTIHWVKQAHGQSLEWIGGINPNNGGTVYNQKFQGKATLTVDKSTSTAYMELRSLTSEDTAVYYCARVRRGMDYWGQGTSVTVSS

SEQ ID NO: 325 - protein, 107 aa, unidentified
DIQMTQSPKSLSTSVGDRVTITCRASQDVTTAVAWYQQKPGKSPKLLIYSASYRYTGVPSRFSGSGSGTDFTFTISSVQPEDLATYYCQQHYSAPWTFGGGTNLEIK

SEQ ID NO: 326 - protein, 116 aa, unidentified
QVQLVQSGAEVVKPGASVKVSCKTSGYTFTEYTIHWVRQAPGQSLEWIGGINPNNGGTVYNQKFQGKVTLTVDKSTSTAYMELSSLRSEDTAVYYCARVRRGMDYWGQGTSVTVSS

SEQ ID NO: 327 - protein, 107 aa, unidentified
DIQMTQSPSSLSASVGDRVTITCRASQDVTTAVAWYQQKPGKAPKLLIYSASYRYTGVPSRFSGSGSGTDFTLTISSVQPEDLATYYCQQHYSAPWTFGGGTNLEIK

SEQ ID NO: 328 - protein, 117 aa, unidentified
EVQLVESGGGLVQPGGSLRLSCAASGIDLRTNSMSWVRQAPGKGLEWVGIIGSSGYMDYASWAKGRFTISRDNSKNTLYLQMNSLRAEDTAVYYCARHGDSSSFALWGQGTLVTVSS

SEQ ID NO: 329 - protein, 110 aa, unidentified
DIQMTQSPSSLSASVGDRVTITCRASESVYKNNRLSWYQQKPGKAPKLLIYEASKVASGVPSRFSGSGSGTDFTLTISSLQPEDFATYYCAGAYRGNIYPFGQGTKVEIK

SEQ ID NO: 330 - protein, 116 aa, unidentified
EVQLVESGGGLVQPGGSLRLSCAASGFSLNSNAVGWVRQAPGKGLEYIGFINFSGITYYANWAKGRFTISRDNSKNTLYLQMNSLRAEDTAVYYCARGSAGSFDLWGQGTLVTVSS

SEQ ID NO: 331 - protein, 110 aa, unidentified
DIQMTQSPSSLSASVGDRVTITCQASESVYKNNYLAWYQQKPGKAPKLLIYRASNLASGVPSRFSGSGSGTDFTLTISSLQPEDFATYYCQGGYSSGMYPFGQGTKVEIK

SEQ ID NO: 332 - protein, 118 aa, unidentified
EVQLVESGGGLVKPGGSLKLSCAASGFTFSDYGMHWVRQAPEKGLEWVAFISSGSSIIYYADIVKGRSTISRDNAKNTLFLQMTSLRSEDTAMYYCTRGNKRGFFDYWGQGTILTVSS

SEQ ID NO: 333 - protein, 108 aa, unidentified
QIVLTQSPALMSASPGEKVTMTCNASSSVSYMYWYQRKPRSSPKPWIYLTSNLASGVPVRFSGSGSGTSYSLTISSMEAEDAATYYCQQWRSNPPMLTFGSGTKLEVK

SEQ ID NO: 334 - protein, 118 aa, unidentified
EVQLVESGGGLVQPGGSLRLSCAASGFTFSDYGMHWVRQAPGKGLEWVAFISSGSSIIYYADIVKGRSTISRDNAKNTLYLQMNSLRAEDTAVYYCTRGNKRGFFDYWGQGTLVTVSS

SEQ ID NO: 335 - protein, 108 aa, unidentified
EIVLTQSPATLSLSPGERATLSCRASSSVPYMYWYQQKPGQAPRLLIYLTSNLASGIPARFSGSGSGTDFTLTISSLEPEDFAVYYCQQWRAYPPMLTFGGGTKVEIK

SEQ ID NO: 336 - protein, 108 aa, unidentified
EIVLTQSPATLSLSPGERATLSCRASPGVPPLAWYQQKPGQAPRLLIYLASSRPSGIPARFSGSGSGTDFTLTISSLEPEDFAVYYCQQWRSNPPMLTFGGGTKVEIK

SEQ ID NO: 337 - protein, 122 aa, unidentified
EVQLVESGGGLVKPGGSLKLSCAASGFTFSDYGMHWVRQAPEKGLEWVAYISSGSTTIYYADTVKGRFTISRDNGKNTLFLQMTSLRSEDTAMYYCARISTVVAKRYAMDYWGQGTSVTVSS

SEQ ID NO: 338 - protein, 107 aa, unidentified
DIQMTQTASSLSASLGDRVTISCRASQDISNYLNWYQQKPDGTVKLLIYYTSRLHSGVPSRFSGSGSGTDYSLTISNLEQEDIATYFCQQINALPLTFGAGTKLELK

SEQ ID NO: 339 - protein, 122 aa, unidentified
EVQLVESGGGLVQPGGSLRLSCAASGFTFSDYGMHWVRQAPGKGLEWVSYISSGSTTIYYADTVKGRFTISRDNAKNSLYLQMNSLRAEDTAVYYCARISTVVAKRYAMDYWGQGTLVTVSS

SEQ ID NO: 340 - protein, 107 aa, unidentified
DIQMTQSPSSLSASVGDRVTITCRASQDISNYLNWYQQKPGKAPKLLIYYTSRLHSGVPSRFSGSGSGTDFTLTISSLQPEDIATYYCQQINALPFTFGQGTKLEIK

SEQ ID NO: 341 - protein, 125 aa, unidentified
EVQLVESGGGLVQPGGSLRLSCAVSGFTFSSYAMSWVRQAPGKGLEWVSSITGGGGGIYYADSVKGRFTISRDNSKNTVYLQMNSLRAEDTAVYYCAKDRISITIRPRYFGLDFWGQGTTVTVSS

SEQ ID NO: 342 - protein, 112 aa, unidentified
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQSFKAPYTFGQGTKLEIK

SEQ ID NO: 343 - protein, 112 aa, unidentified
DIVMTQSPLSLPVTPGEPASISCRSSRNVIYGNGYNYLDWYLQKSGQSPQLLIYLGNNVAAGVPDRFSGSGSGTDFTLKISRVEAEDVGFYYCMQSLQAPYTFGQGTKLEIK

SEQ ID NO: 344 - protein, 112 aa, unidentified
DIVMTQSPLSLPVTPGEPASISCRSSQNVVYGNGYNYLDWYLQKSGQSPQLLIYLGTNVAAGVPDRFSGSGSGTDFTLKISRVEAEDVGFYYCMQSLQAPYTFGQGTKLEIK

SEQ ID NO: 345 - protein, 112 aa, unidentified
DIVMTQSPLSLPVTPGEPASISCRSSQNVVYGNGYNYLDWYLQKSGQSPQLLIYLGNNVAAGVPDRFSGSGSGTDFTLKISRVEAEDVGFYYCMQSLKAPYTFGQGTKLEIK

SEQ ID NO: 346 - protein, 112 aa, unidentified
DIVMTQSPLSLPVTPGEPASISCRSSHNLLYSNGYNYLDWYLQKPGQSPQLLIYLGSNRAYGVPDRFSGSGSGTDFTLKISRVEAEDVGVYHCMQALQSPYTFGQGTKVDIK

SEQ ID NO: 347 - protein, 112 aa, unidentified
DIVMTQSPLSLPVTAGAPASISCRSSQSLLYSNGYNYLDWYMQKPGQSPQLLIYLGSNRASGVPDRFSGSGSGTDFTLKISRVEAEDVGVYYCMQALETPYAFGQGTKVEIK

SEQ ID NO: 348 - protein, 120 aa, unidentified
QVQLQQSGPELVKPGASVKISCTASGSTLTDDYINWVKQRPGRGLEWVGWIFPGNGNSYYNEKFKDKATLIVDKSSSTAYMLLSSLTSEDSAVYFCARGLVRYRALFDYWGQGTTLTVSS

SEQ ID NO: 349 - protein, 106 aa, unidentified
QIVLSQSPAILSASPGEKVTMTCRASSSINYMHWYQQKPGSSPKPWIYAASNLASGVPARFSGSGSGTSFSLTISRVEAEDAATYYCQQWSSYPITFGSGTKLEIK

SEQ ID NO: 350 - protein, 120 aa, unidentified
QVQLVQSGAEVKKPGASVKVSCKASGSTLTDDYINWVRQAPGQRLEWVGWIFPGNGNSYYNEKFKDRATLTVDKSASTAYMELSSLRSEDTAVYFCARGLVRYRALFDYWGQGTLVTVSS

SEQ ID NO: 351 - protein, 106 aa, unidentified
DIQMTQSPSSLSASVGDRVTITCRASSSINYMHWYQQKPGKAPKPWIYAASNLASGVPSRFSGSGSGTDFTLTISSLQPEDFATYYCQQWSSYPITFGQGTKVEIK

SEQ ID NO: 352 - protein, 119 aa, unidentified
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARGLTTVRGVLYWGQGTLVTVSS

SEQ ID NO: 353 - protein, 107 aa, unidentified
EIVMTQSPSSLSASLGDRVTITCRASQNIGSRLAWYQQKPGKAPKLLIYKASSLESGVPSRFSGSGSGTEFTLTISSLQPDDFATYYCQQYNSYSWTFGQGTKLEIK

SEQ ID NO: 354 - protein, 119 aa, unidentified
EVQLVESGGGLVQPGGSLRLSCAASGFTFSTYGMSWVRQAPGKGLVXVXTINSNGGSTSYPDSVKGRFTISRDNAKNTLYLQMNSLRAEDTAVYYCARFFRFRNAMDYWGQGTLVTVSS

SEQ ID NO: 355 - protein, 112 aa, unidentified
DIVMTQTPLSLSVTPGQPASISCRSSQSIVHSNGNTYLEWYLQKPGQSPQLLIYKVTNRFSGVPDRFSGSGSGTDFTLKISRVEAEDVGVYYCFQGSHVPYTFGQGTKLEIK

SEQ ID NO: 356 - protein, 116 aa, unidentified
EVQLVQSGAEVKKPGATVKISCKVSGFNIKDTYMHWVQQAPGKGLEWMGLIDPTNGYTIYAEKFQGRVTITADTSTDTAYMELSSLRSEDTAVYYCVSRRPWFAYWGQGTLVTVSS

SEQ ID NO: 357 - protein, 116 aa, unidentified
QVQLQQSGAELVKPGASVKLSCTASGFNIKDTYMHWVKQRPEQGLEWVGRIDPTNGYTIYASKFQGKATITADTSSNTAYMQLSSLTSEDTAVYHCVSRRPWFAYWGQGTTLTVSS

SEQ ID NO: 358 - protein, 120 aa, unidentified
EVQLVESGGGLVKPGGSLKLSCAASGFTFSSYAMSWVRQTPEKRLEWVAGIRSGGSYTYYPDTVKGRFTISRDNARNTLYLQMNSLRSEDTAIYYCARGDKLRPYHFDYWGQGTTLTVSS

SEQ ID NO: 359 - protein, 107 aa, unidentified
DIVMTQSHKFMSTSVGDKVSITCKASQDVTTAVAWYQQKPGQSPKLLIYSASYRYTGVPDRFAGSGSGTDFTVTISTVQAEDLAVYYCQQHYSDPYTFGGGTKLEIK

SEQ ID NO: 360 - protein, 120 aa, unidentified
EVQLVESGGGLVKPGGSLKLSCAASGFTFSNYAMSWVRQTPEKRLEWVAGIRSGGSYTYYPDTVKGRFTISRDNARNTLYLQMSSLRSEDTAIYYCARGDKLRPYHFDYWGQGTTLTVSS

SEQ ID NO: 361 - protein, 107 aa, unidentified
DIVMTQSHKFMSTSVGDRVSITCKASQDVSTAVVWYQQKPGQSPKLLIYSASYRYTGVPDRFTGSGSGTDFTFTIITVQAEDLAVYYCQQHYSAPYTFGGGTQLEIK

SEQ ID NO: 362 - protein, 115 aa, unidentified
EVKLVESGGGLVKPGGSLKLSCAASGFTFSSYAMSWVRQTPEKRLEWVASISSGDSTYYLDSVKGRFTISRDNARNILYLQVSSLRSEDTAMYYCERSGGSAPYWGQGTLVTVSA

SEQ ID NO: 363 - protein, 106 aa, unidentified
QIVLTQSPAIMSASPGDMVTISCSASSSVNYMYWYQQKPGSSPKPWIYRTSNLASGVPARFSGSGSGTSYSLTISSMEAEDAATYYCQQYHSFPLTFGAGTKLELK

SEQ ID NO: 364 - protein, 115 aa, unidentified
EVKLVESGGGLVKPGGSLKLSCAASGFTFSSYAMSWVRQTPEKRLEWVASISSGDSTYYLDSVKGRFTISRDNAMNILYLQMSSLRSEDTAVYYCERSGGSAPYWGQGTLVSVSA

SEQ ID NO: 365 - protein, 106 aa, unidentified
QIVLTQSPAIMSASPGEMVTISCSASSSVSYMYWYQQKPGSSPKPWIYRTSNLASGVPARFSGSGSGTSYSLTISSMEAEDAATYYCQQYHSFPLTFGAGTKLELK

SEQ ID NO: 366 - protein, 125 aa, unidentified
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSS

SEQ ID NO: 367 - protein, 8 aa, unidentified
GFTFSNFW

SEQ ID NO: 368 - protein, 8 aa, unidentified
MNQDGSEK

SEQ ID NO: 369 - protein, 18 aa, unidentified
ARDREYCISTSCYDDFDY

SEQ ID NO: 370 - protein, 124 aa, unidentified
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSS

SEQ ID NO: 371 - protein, 8 aa, unidentified
GFTFDDYS

SEQ ID NO: 372 - protein, 8 aa, unidentified
ISWNSGSK

SEQ ID NO: 373 - protein, 17 aa, unidentified
AKYGSGYGKFYHYGLDV

SEQ ID NO: 374 - protein, 124 aa, unidentified
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSS

SEQ ID NO: 375 - protein, 8 aa, unidentified
GFTFDDYS

SEQ ID NO: 376 - protein, 8 aa, unidentified
ISWNSGSI

SEQ ID NO: 377 - protein, 17 aa, unidentified
AKYGSGYGKFYYYGMDV

SEQ ID NO: 378 - protein, 108 aa, unidentified
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 379 - protein, 6 aa, unidentified
QSISSY

SEQ ID NO: 380 - skipped

SEQ ID NO: 381 - protein, 10 aa, unidentified
QQSYSTPPIT

SEQ ID NO: 382 - protein, 451 aa, unidentified
EVQLVESGGGLVQPGGSLRLSCAASGFTFSNFWMTWVRQAPGKGLEWVANMNQDGSEKYYVDSVKGRFTISRDNAKSSLYLQMNSLRAEDTAVYYCARDREYCISTSCYDDFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 383 - protein, 450 aa, unidentified
EVQLVESGGGLVQPGRSLRLSCAASGFTFDDYSMHWVRQAPGKGLEWVSGISWNSGSKGYADSVKGRFTISRDNAKNSLYLQMNSLRAEDTALYYCAKYGSGYGKFYHYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 384 - protein, 451 aa, unidentified; identical to SEQ ID NO: 382

SEQ ID NO: 385 - protein, 450 aa, unidentified
EVQLVESGGGLVQPGRSLRLSCAASGFTFDDYSMHWVRQAPGKGLEWVSGISWNSGSIGYADSVKGRFTISRDNAKNSLYLQMNSLRAEDTALYYCAKYGSGYGKFYYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSPGK

SEQ ID NO: 386 - protein, 215 aa, unidentified
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 387 - protein, 451 aa, unidentified
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

(19) United States
(12) Patent Application Publication
(10) Pub. No.: US 2026/0201051 A1
(43) Pub. Date: Jul. 16, 2026

(54) Methods of Treating Food Allergy by Administering an IL-4R Antagonist and a BCMA Targeting Agent

(71) Applicant: Regeneron Pharmaceuticals, Inc., Tarrytown, NY (US)

(72) Inventors: Andre Limnander, New York, NY (US); Jennifer Maloney, Hastings-on-Hudson, NY (US); Andrew J. Murphy, Croton-on-Hudson, NY (US); Jamie M. Orengo, Cortlandt Manor, NY (US); Odelya E. Pagovich, New York, NY (US); Allen Radin, New York, NY (US); George D. Yancopoulos, Yorktown Heights, NY (US)

(21) Appl. No.: 19/444,955

(22) Filed: Jan. 9, 2026

Related U.S. Application Data
(60) Provisional application No. 63/743,821, filed on Jan. 10, 2025.

(51) Int. Cl. C07K 16/28; A61K 39/00; A61P 37/08

(52) U.S. Cl. CPC C07K 16/2878; A61P 37/08; C07K 16/2809; C07K 16/2866; A61K 2039/507; A61K 2039/545; C07K 2317/31; C07K 2317/565; C07K 2317/76

(57) ABSTRACT

Methods for treating a subject having an IgE-mediated allergy or for preventing or reducing the severity of an allergic reaction in a subject having an IgE-mediated allergy are provided. In one aspect, the methods comprise administering to a subject having an IgE-mediated allergy an IL-4R antagonist, such as an IL-4Rα antibody, and a BCMA targeting agent, such as a BCMA x CD3 bispecific antibody.

### CROSS-REFERENCE TO RELATED APPLICATIONS

This application claims priority to U.S. Provisional Patent Application No. 63/743,821, filed Jan. 10, 2025, the entire content of which is incorporated by reference herein.

### REFERENCE TO A SEQUENCE LISTING

This application incorporates by reference a computer readable Sequence Listing in ST.26 XML format, titled 11933US01_Sequence, created on Jan. 9, 2026, and containing 374,292 bytes.

### FIELD OF THE INVENTION

The present disclosure relates to the use of interleukin-4 receptor (IL-4R) antagonists in combination with B-cell maturation antigen (BCMA) targeting agents, such as bispecific antibodies that bind BCMA and CD3, for reducing serum IgE and treating IgE-mediated allergies such as food allergy.

### BACKGROUND

Food allergy is a potentially life-threatening condition that affects between 10% to 13% of adults in the US and has been increasing in prevalence over the last 2 to 3 decades. Among US adults, data shows that shellfish allergy is the most common (~2.9%), followed by milk, peanut, tree nuts, and fin fish (Dyer et al., Allergy Asthma Proc 2015, 36:58-64). Unlike milk and egg allergy that are common in childhood, peanut and tree nut allergies typically persist into adulthood and are associated with a higher incidence of severe anaphylaxis versus other food allergies. Factors more common in people that will not outgrow their food allergies include history of multiple food-induced anaphylaxis events, increased number of anaphylactic reactions to the same food, increased number of anaphylactic reactions per patient, and more robust sensitization to the food (Buyuk Yaytokgil et al., Allergy Asthma Proc 2022, 43:57-63). Persistent childhood-onset allergies—especially to milk, egg, and wheat—in adults can severely limit diet, function, and quality of life (Patel et al., Ann Allergy Asthma Immunol 2021, 127:70-75.e2; Sicherer et al., J Allergy Clin Immunol Pract 2020, 8:1854-1864). Adults may also develop new food allergies, and evidence suggests that certain food allergies (e.g., shellfish and fin fish) are more likely than others to develop during adulthood. Moreover, studies suggest that rates of food allergy related emergency department visits may be increasing among young adults, and food allergy is associated with a significant psychosocial burden for patients and families, which can lead to social limitations, hypervigilance, and anxiety.

Food allergy is mediated by type I hypersensitivity responses linked to antigen-specific IgE. The immune system in the gut actively induces an immune tolerant state to the proteins that are normally consumed. Food allergy occurs when the body has a break in this tolerance, which results in an abnormal immune reaction to food. Crosslinking by allergen of allergen-specific IgE bound to high-affinity IgE receptors on mast cells and basophils triggers immediate degranulation. Subsequent release of a diverse array of inflammatory mediators results in severe allergic symptoms such as hives, wheezing, vomiting and, in severe cases, anaphylactic shock and sometimes death after allergen ingestion. Release of these mediators also initiates type 2 helper cell cytokine release, which results in eosinophil infiltration and creates a vicious cycle of chronic allergic inflammation.

For patients with peanut allergy, PALFORZIA™ oral immunotherapy (OIT) to peanut is an approved therapy which involves a slow up-dosing of exposure to allergen over time to desensitize or increase the threshold of reactivity to a given food; typically upon reaching a target level of peanut protein, patients are continued on a maintenance dose of peanut protein to maintain desensitization. Although some patients on a maintenance dose of peanut protein have demonstrated desensitization to peanut, up to 80% of patients exhibit related adverse events during OIT, with 42% experiencing systemic reactions and 49% experiencing gastrointestinal symptoms (Virkud et al., J Allergy Clin Immunol 2017, 139:882-888.e5). Because PALFORZIA™ is associated with a risk of anaphylaxis which may be life-threatening and can occur at any time, it is available only through a restricted access program, and patients are required to be instructed and trained on the appropriate use of epinephrine and to seek immediate medical care upon use of epinephrine. An additional limitation with OIT is its limited ability to induce clinical tolerance when patients are taken off daily peanut intake (Vickery et al., J Allergy Clin Immunol 2014, 133:468-475). Even with years of immunotherapy, many patients never achieve tolerance and are re-sensitized within weeks of halting daily peanut intake, with a small percentage (~10%) maintaining a sustained unresponsiveness even after 3 months off OIT (Narisety et al., J Allergy Clin Immunol 2015, 135:1275-1282.e1-6). Furthermore, there are no other FDA-approved OIT treatments aside from peanut OIT, which results in significant limitations to this treatment paradigm given that patients may be allergic to many other foods.

Anti-IgE therapy prevents the binding of IgE to FcεR1 and decreases surface FcεR1 on mast cells, basophils, and dendritic cells, thus preventing allergen induced crosslinking of IgE-FcεR1 complexes and subsequent degranulation of mast cells and basophils (Holgate et al., Clin Exp Allergy 2005, 35:408-416). Omalizumab, a humanized anti-IgE monoclonal antibody, binds to IgE Cε3 domains, outside of the FcεR1-binding site, and sterically disrupts binding to both FcεR1 and CD23 (Pennington et al., Nat Commun 2016, 7:11610). In food allergy, several clinical trials showed the safety and usefulness in inhibiting allergic responses with omalizumab as monotherapy, or in association with allergen-specific immunotherapy (Lieberman et al., Curr Allergy and Asthma Rep 2013, 13:78-84). Other high-affinity anti-IgE antibodies like ligelizumab are being studied for food allergy (Wood et al., World Allergy Organ J 2022, 15:100690). However, using an anti-IgE antibody as prophylaxis against food-induced anaphylaxis has challenges, because the protection provided by a single subcutaneous administration is short, estimated at 2 to 4 weeks, requiring at least monthly parenteral administration of the antibody to maintain persistent effective therapy. Furthermore, neither omalizumab nor ligelizumab target the source of IgE production nor induce tolerance to allergens (Wood et al., New Engl J Med 2024, 390:889-899).

Thus, there remains a high unmet need to develop therapies for IgE-mediated food allergy.

### SUMMARY

In one aspect, the present disclosure provides methods of treating a subject having an IgE-mediated allergy or preventing or reducing the severity of an allergic reaction in a subject having an IgE-mediated allergy. In some embodiments, the method comprises:
administering to the subject an interleukin-4 receptor (IL-4R) antagonist, wherein the IL-4R antagonist comprises an antibody that specifically binds IL-4Rα or an antigen-binding fragment thereof; and
administering to the subject a B-cell maturation antigen (BCMA) targeting agent, wherein the BCMA targeting agent comprises a bispecific antigen-binding molecule comprising a first antigen-binding domain that specifically binds BCMA and a second antigen-binding domain that specifically binds CD3;
wherein prior to administering the BCMA targeting agent, the IL-4R antagonist is administered to the subject for a length of time sufficient to attain a steady state concentration of the IL-4R antagonist in the subject.

In another aspect, the present disclosure provides methods for reducing serum total IgE in a subject having an IgE-mediated allergy. In some embodiments, the method comprises:
administering to the subject an interleukin-4 receptor (IL-4R) antagonist, wherein the IL-4R antagonist comprises an antibody that specifically binds IL-4Rα or an antigen-binding fragment thereof; and
administering to the subject a BCMA targeting agent, wherein the BCMA targeting agent comprises a bispecific antigen-binding molecule comprising a first antigen-binding domain that specifically binds BCMA and a second antigen-binding domain that specifically binds CD3;
wherein prior to administering the BCMA targeting agent, the IL-4R antagonist is administered to the subject for a length of time sufficient to attain a steady state concentration of the IL-4R antagonist in the subject.

In another aspect, the present disclosure provides methods for reducing allergen-specific IgE in a subject having an IgE-mediated allergy. In some embodiments, the method comprises:
administering to the subject an interleukin-4 receptor (IL-4R) antagonist, wherein the IL-4R antagonist comprises an antibody that specifically binds IL-4Rα or an antigen-binding fragment thereof; and
administering to the subject a BCMA targeting agent, wherein the BCMA targeting agent comprises a bispecific antigen-binding molecule comprising a first antigen-binding domain that specifically binds BCMA and a second antigen-binding domain that specifically binds CD3;
wherein prior to administering the BCMA targeting agent, the IL-4R antagonist is administered to the subject for a length of time sufficient to attain a steady state concentration of the IL-4R antagonist in the subject.

In some embodiments, the IL-4R antagonist is administered to the subject for at least 12 weeks prior to administering the BCMA targeting agent. In some embodiments, the IL-4R antagonist is administered to the subject for at least 14 weeks, at least 16 weeks, at least 18 weeks, or at least 20 weeks prior to administering the BCMA targeting agent.

In some embodiments, the subject has an IgE-mediated food allergy. In some embodiments, the subject has an allergy to one or more of peanut, hazelnut, walnut, cashew, milk, egg, egg white, soy, wheat, sesame, cod, salmon, tuna, lobster, crab, and shrimp. In some embodiments, the subject has an allergy to multiple foods. In some embodiments, the subject has an IgE-mediated allergy to one or more perennial allergens.

In some embodiments, the subject is an adult.

In some embodiments, the subject has a baseline serum total IgE ≥150 kU/L.

In some embodiments, at least two doses of the BCMA targeting agent are administered to the subject. In some embodiments, the BCMA targeting agent is administered to the subject once weekly (QW). In some embodiments, no more than 7 doses of the BCMA targeting agent are administered to the subject. In some embodiments, a single dose of the BCMA targeting agent is administered to the subject.

In some embodiments, the BCMA targeting agent is administered at a dose of from about 0.15 mg to about 80 mg; or is administered at a dose of about 0.5 mg; or is administered at a dose of about 1.5 mg; or is administered at a dose of about 5 mg; or is administered at a dose of about 10 mg; or is administered at a dose of about 20 mg; or is administered at a dose of about 40 mg; or is administered at a dose of about 80 mg.

In some embodiments, the IL-4R antagonist is administered to the subject at a dose of 50 mg to 600 mg. In some embodiments, the IL-4R antagonist is administered to the subject at a dose of 200 mg or 300 mg. In some embodiments, the IL-4R antagonist is administered to the subject once weekly (QW) or once every two weeks (Q2W). In some embodiments, the IL-4R antagonist is administered to the subject as an initial dose followed by one or more secondary doses, wherein: the IL-4R antagonist is administered as an initial dose of 400 mg followed by one or more secondary doses of 200 mg; or the IL-4R antagonist is administered as an initial dose of 600 mg followed by one or more secondary doses of 300 mg. In some embodiments, each secondary dose is administered one week or two weeks after the immediately preceding dose.

In some embodiments, the anti-IL-4Rα antibody or antigen-binding fragment thereof comprises three heavy chain complementarity determining regions (HCDR1, HCDR2 and HCDR3) contained within a heavy chain variable region (HCVR) comprising the amino acid sequence of SEQ ID NO:1, and three light chain complementarity determining regions (LCDR1, LCDR2 and LCDR3) contained within a light chain variable region (LCVR) comprising the amino acid sequence of SEQ ID NO:2. In some embodiments, the anti-IL-4Rα antibody or antigen-binding fragment thereof comprises an HCDR1 comprising the amino acid sequence of SEQ ID NO:3, an HCDR2 comprising the amino acid sequence of SEQ ID NO:4, an HCDR3 comprising the amino acid sequence of SEQ ID NO:5, an LCDR1 comprising the amino acid sequence of SEQ ID NO:6, an LCDR2 comprising the amino acid sequence of SEQ ID NO:7, and an LCDR3 comprising the amino acid sequence of SEQ ID NO:8. In some embodiments, the anti-IL-4Rα antibody or antigen-binding fragment thereof comprises an HCVR comprising the amino acid sequence of SEQ ID NO:1 and an LCVR comprising the amino acid sequence of SEQ ID NO:2. In some embodiments, the anti-IL-4Rα antibody is a full antibody. In some embodiments, the full antibody is an IgG antibody. In some embodiments, the full antibody is an IgG4 antibody. In some embodiments, the anti-IL-4Rα antibody comprises a heavy chain comprising the amino acid sequence of SEQ ID NO:9 or SEQ ID NO:387 and a light chain comprising the amino acid sequence of SEQ ID NO:10. In some embodiments, the IL-4R antagonist is dupilumab.

In some embodiments, the IL-4R antagonist is AMG317, APG808, BA2101, comekibart, manfidokimab, LQ036, MEDI 9314, NS-402, QX-005N, SHR-1819, rademikibart, stapokibart, or TQH-2722.

In some embodiments, the BCMA targeting agent is an anti-BCMA x anti-CD3 bispecific antibody or antigen-binding fragment thereof comprising a first antigen-binding domain that specifically binds BCMA and a second antigen-binding domain that specifically binds CD3;
wherein the first antigen-binding domain comprises the HCDRs of an HCVR comprising the amino acid sequence of SEQ ID NO:366 and the LCDRs of an LCVR comprising the amino acid sequence of SEQ ID NO:378; and
wherein the second antigen-binding domain comprises the HCDRs of an HCVR comprising the amino acid sequence of SEQ ID NO:370 or SEQ ID NO:374 and the LCDRs of an LCVR comprising the amino acid sequence of SEQ ID NO:378.

In some embodiments, the first antigen-binding domain comprises an HCDR1 comprising the amino acid sequence of SEQ ID NO:367, an HCDR2 comprising the amino acid sequence of SEQ ID NO:368, an HCDR3 comprising the amino acid sequence of SEQ ID NO:369, an LCDR1 comprising the amino acid sequence of SEQ ID NO:379, an LCDR2 comprising the amino acid sequence of SEQ ID NO:380, and an LCDR3 comprising the amino acid sequence of SEQ ID NO:381; and wherein the second antigen-binding domain comprises an HCDR1 comprising the amino acid sequence of SEQ ID NO:371, an HCDR2 comprising the amino acid sequence of SEQ ID NO:372, an HCDR3 comprising the amino acid sequence of SEQ ID NO:373, an LCDR1 comprising the amino acid sequence of SEQ ID NO:379, an LCDR2 comprising the amino acid sequence of SEQ ID NO:380, and an LCDR3 comprising the amino acid sequence of SEQ ID NO:381. In some embodiments, the first antigen-binding domain comprises an HCVR comprising the amino acid sequence of SEQ ID NO:366 and an LCVR comprising the amino acid sequence of SEQ ID NO:378; and wherein the second antigen-binding domain comprises an HCVR comprising the amino acid sequence of SEQ ID NO:370 and an LCVR comprising the amino acid sequence of SEQ ID NO:378. In some embodiments, the first antigen-binding domain comprises a heavy chain comprising the amino acid sequence of SEQ ID NO:382 and a light chain comprising the amino acid sequence of SEQ ID NO:386; and wherein the second antigen-binding domain comprises a heavy chain comprising the amino acid sequence of SEQ ID NO:383 and a light chain comprising the amino acid sequence of SEQ ID NO:386. In some embodiments, the BCMA targeting agent is linvoseltamab.

In some embodiments, the first antigen-binding domain comprises an HCDR1 comprising the amino acid sequence of SEQ ID NO:367, an HCDR2 comprising the amino acid sequence of SEQ ID NO:368, an HCDR3 comprising the amino acid sequence of SEQ ID NO:369, an LCDR1 comprising the amino acid sequence of SEQ ID NO:379, an LCDR2 comprising the amino acid sequence of SEQ ID NO:380, and an LCDR3 comprising the amino acid sequence of SEQ ID NO:381; and wherein the second antigen-binding domain comprises an HCDR1 comprising the amino acid sequence of SEQ ID NO:375, an HCDR2 comprising the amino acid sequence of SEQ ID NO:376, an HCDR3 comprising the amino acid sequence of SEQ ID NO:377, an LCDR1 comprising the amino acid sequence of SEQ ID NO:379, an LCDR2 comprising the amino acid sequence of SEQ ID NO:380, and an LCDR3 comprising the amino acid sequence of SEQ ID NO:381. In some embodiments, the first antigen-binding domain comprises an HCVR comprising the amino acid sequence of SEQ ID NO:366 and an LCVR comprising the amino acid sequence of SEQ ID NO:378; and wherein the second antigen-binding domain comprises an HCVR comprising the amino acid sequence of SEQ ID NO:374 and an LCVR comprising the amino acid sequence of SEQ ID NO:378. In some embodiments, the first antigen-binding domain comprises a heavy chain comprising the amino acid sequence of SEQ ID NO:384 and a light chain comprising the amino acid sequence of SEQ ID NO:386; and wherein the second antigen-binding domain comprises a heavy chain comprising the amino acid sequence of SEQ ID NO:385 and a light chain comprising the amino acid sequence of SEQ ID NO:386. In some embodiments, the BCMA targeting agent is vonsetamig.

In some embodiments, the BCMA targeting agent is alnuctamab, elranatamab, pacanalotamab, pavurutamab, teclistamab, CM336, EM801, EMB-06, HBM7020, TNB-383B, TNB-384B, TQB2934, or YKST02.

In some embodiments, the level of serum total IgE in the subject is reduced by at least 50%. In some embodiments, the level of serum total IgE in the subject is reduced to <2 kU/L. In some embodiments, after administering the BCMA targeting agent to the subject, the method further comprises determining the concentration of total IgE in a serum sample from the subject; wherein a further dose of the BCMA targeting agent is administered to the subject only if the subject has a serum total IgE concentration ≥2 kU/L.

In another aspect, the disclosure provides methods of treating a subject having an IgE-mediated food allergy or preventing or reducing the severity of an allergic reaction in a subject having an IgE-mediated food allergy, comprising:
administering to the subject an antibody that specifically binds IL-4Rα, wherein the antibody is dupilumab; and
administering to the subject an anti-BCMA x anti-CD3 bispecific antibody or an antigen-binding fragment thereof;
wherein prior to administering the anti-BCMA x anti-CD3 bispecific antibody or an antigen-binding fragment thereof, the dupilumab is administered to the subject for at least 12 weeks, wherein the dupilumab is administered: (i) at a dosage of 300 mg every week (QW) or every two weeks (Q2W); or (ii) at an initial dose of 600 mg followed by one or more secondary doses of 300 mg, wherein each secondary dose is administered one week or two weeks after the immediately preceding dose.

In some embodiments, the anti-BCMA x anti-CD3 bispecific antibody is alnuctamab, elranatamab, linvoseltamab, pacanalotamab, pavurutamab, teclistamab, vonsetamig, CM336, EM801, EMB-06, HBM7020, TNB-383B, TNB-384B, TQB2934, or YKST02. In some embodiments, the anti-BCMA x anti-CD3 bispecific antibody is linvoseltamab. In some embodiments, the anti-BCMA x anti-CD3 bispecific antibody is vonsetamig.

In some embodiments, the subject has an allergy to one or more of peanut, hazelnut, walnut, cashew, milk, egg, egg white, soy, wheat, sesame, cod, salmon, tuna, lobster, crab, and shrimp. In some embodiments, the subject has an allergy to multiple foods. In some embodiments, the subject has an allergy to one or more perennial allergens.

In some embodiments, the subject has a baseline serum total IgE ≥150 kU/L.

In some embodiments, the BCMA targeting agent is administered at a dose of from about 0.15 mg to about 80 mg. In some embodiments, at least two doses of the BCMA targeting agent are administered to the subject. In some embodiments, the BCMA targeting agent is administered to the subject once weekly (QW). In some embodiments, a single dose of the BCMA targeting agent is administered to the subject.

In another aspect, the disclosure provides an interleukin-4 receptor (IL-4R) antagonist for use in methods of treating a subject having an IgE-mediated allergy or preventing or reducing the severity of an allergic reaction in a subject having an IgE-mediated allergy, or for use in methods of reducing serum total IgE in a subject having an IgE-mediated allergy, or use in methods of reducing allergen-specific IgE in a subject having an IgE-mediated allergy. In some embodiments, the IL-4R antagonist comprises an antibody that specifically binds IL-4Rα or an antigen-binding fragment thereof. In some embodiments, the IL-4R antagonist is administered to the subject for a length of time sufficient to attain a steady state concentration of the IL-4R antagonist in the subject prior to administering to the subject a BCMA targeting agent. In some embodiments, the BCMA targeting agent comprises a bispecific antigen-binding molecule comprising a first antigen-binding domain that specifically binds BCMA and a second antigen-binding domain that specifically binds CD3.

In another aspect, the disclosure provides compositions comprising: (i) an IL-4R antagonist that comprises an antibody that specifically binds IL-4Rα or an antigen-binding fragment thereof; and (ii) a BCMA targeting agent that comprises a bispecific antigen-binding molecule comprising a first antigen-binding domain that specifically binds BCMA and a second antigen-binding domain that specifically binds CD3; for use in treating a subject having an IgE-mediated allergy or preventing or reducing the severity of an allergic reaction in a subject having an IgE-mediated allergy, or reducing serum total IgE in a subject having an IgE-mediated allergy, or reducing allergen-specific IgE in a subject having an IgE-mediated allergy.

In yet another aspect, the disclosure provides combinations comprising: (i) an IL-4R antagonist that comprises an antibody that specifically binds IL-4Rα or an antigen-binding fragment thereof; and (ii) a BCMA targeting agent that comprises a bispecific antigen-binding molecule comprising a first antigen-binding domain that specifically binds BCMA and a second antigen-binding domain that specifically binds CD3; for use in treating a subject having an IgE-mediated allergy or preventing or reducing the severity of an allergic reaction in a subject having an IgE-mediated allergy, or reducing serum total IgE in a subject having an IgE-mediated allergy, or reducing allergen-specific IgE in a subject having an IgE-mediated allergy.

In yet another aspect, the disclosure provides kits for use in methods of treating a subject having an IgE-mediated allergy or preventing or reducing the severity of an allergic reaction in a subject having an IgE-mediated allergy, or for use in methods of reducing serum total IgE in a subject having an IgE-mediated allergy, or use in methods of reducing allergen-specific IgE in a subject having an IgE-mediated allergy. In some embodiments, the kit comprises: (i) an IL-4R antagonist comprises an antibody that specifically binds IL-4Rα or an antigen-binding fragment thereof; and (ii) a BCMA targeting agent that comprises a bispecific antigen-binding molecule comprising a first antigen-binding domain that specifically binds BCMA and a second antigen-binding domain that specifically binds CD3; and optionally (iii) instructions for using the kit to practice the methods disclosed herein.

In another aspect, the disclosure provides an IL-4R antagonist that comprises an antibody that specifically binds IL-4Rα or an antigen-binding fragment thereof; and a BCMA targeting agent that comprises a bispecific antigen-binding molecule comprising a first antigen-binding domain that specifically binds BCMA and a second antigen-binding domain that specifically binds CD3, for use in the preparation of a medicament for treating a subject having an IgE-mediated allergy or preventing or reducing the severity of an allergic reaction in a subject having an IgE-mediated allergy, or reducing serum total IgE in a subject having an IgE-mediated allergy, or reducing allergen-specific IgE in a subject having an IgE-mediated allergy.

Other embodiments will be apparent from a review of the ensuing detailed description.

### BRIEF DESCRIPTION OF THE FIGURES

FIG. 1. Overview of study design for clinical trial disclosed in Example 1. *Observation ≥24 hours in an inpatient monitored setting to allow for early identification and management of IRR/CRS. ¹600 mg SC loading dose followed by 300 mg Q2W SC. A minimum of 12 weeks of dupilumab therapy is required during the run-in period for treatment-naïve participants. ²Linvoseltamab safety follow-up period, 52 weeks from the end of the 30-week combination study treatment period for all participants. ³Linvoseltamab dose levels and further details on intra-participant dose escalation are provided in clinical study protocol. ⁴An additional 6 participants may be enrolled after evaluation of data from the first 6 participants. ⁵300 mg QW SC for participants receiving commercial dupilumab for EoE at screening; 300 mg Q2W SC for participants receiving commercial dupilumab for AD at screening and for participants who are dupilumab-naïve at screening.

FIG. 2A-2C. Study participant #1 IgE (kU/L) levels over time, as measured by a central lab (2A, 2C) and as measured by a local lab (2B). Dotted line is reference range of normal IgE.

FIG. 3. Study participant #1 food-specific IgE (kU/L) levels over time. Reference range for all allergens is <0.35 kU/L.

FIG. 4. Study participant #2 IgE (kU/L) levels over time. Dotted line is reference range of normal IgE.

FIG. 5. Study participant #2 food-specific IgE (kU/L) levels over time. Reference range for all allergens is <0.35 kU/L.

FIG. 6. Study participant #3 IgE (kU/L) levels over time. Dotted line is reference range of normal IgE.

FIG. 7. Study participant #3 food-specific IgE (kU/L) levels over time. Reference range for all allergens is <0.35 kU/L.

### DETAILED DESCRIPTION

### Definitions

Before the present invention is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

The term “antibody,” as used herein, refers to an antigen-binding molecule or molecular complex comprising a set of complementarity determining regions (CDRs) that specifically bind to or interact with a particular antigen (e.g., IL-4R). The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the anti-IL-4R antibody (or antigen-binding portion thereof) are identical to the human germline sequences. In some embodiments, one or more FRs of the anti-IL-4R antibody (or antigen-binding portion thereof) are naturally or artificially modified. In some embodiments, the antibody is a human IgG antibody. In some embodiments, the antibody is a human IgG4 antibody.

The term “antigen-binding fragment,” as used herein, refers to an immunoglobulin (e.g., molecule or complex), other than an intact antibody as provided above, that comprises a portion of an antibody and specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed by the term “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol., 273:927-948 (1997); and Martin et al., Proc. Natl. Acad. Sci. USA, 86:9268-9272 (1989). Public databases are also available for identifying CDR sequences within an antibody. In some embodiments, the CDRs within the HCVR and/or LCVR are identified according to the Kabat definition. In some embodiments, the CDRs within the HCVR and/or LCVR are identified according to the Chothia definition. In some embodiments, the CDRs within the HCVR and/or LCVR are identified according to the AbM definition. In some embodiments, the CDRs within the HCVR and/or LCVR are identified according to the IMGT definition.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (X) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homodimer or heterodimer (or other multimer) of any of the variable and constant domain configurations listed above in non-covalent association with one another and/or with one or more monomeric V_{H} or V_{L} domain (e.g., by disulfide bond(s)).

The constant region of an antibody is important in the ability of an antibody to fix complement and mediate cell-dependent cytotoxicity. Thus, in some embodiments the isotype of an antibody may be selected on the basis of whether it is desirable for the antibody to mediate cytotoxicity.

As used herein, a “multispecific antibody” refers to an antibody at least two different variable domains, wherein each variable domain is capable of specifically binding to a separate antigen or to a different epitope on the same antigen. In some embodiments, a multispecific antibody is a bispecific antibody. Any multispecific antibody format may be adapted for use in the context of the present disclosure using routine techniques available in the art. For example, in some embodiments the methods of the present disclosure comprise the use of bispecific antibodies wherein one arm of an immunoglobulin is specific for IL-4Rα or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein, et al., 2012, mAbs, 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane, et al., J. Am. Chem. Soc. [Epub: Dec. 4, 2012]).

The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor et al., (1992) Nucl. Acids Res., 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, an “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

As used herein, the term “specifically binds,” as used herein, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. In some embodiments, an antibody that “specifically binds” to a target antigen binds to that antigen, or a portion thereof, with an equilibrium dissociation constant (K_{D}) of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 0.5 nM, less than about 0.25 nM, less than about 0.1 nM or less than about 0.05 nM, as measured in a surface plasmon resonance assay (e.g., BIAcore™, Biacore Life Sciences division of GE Healthcare, Piscataway, NJ). In some embodiments, an antibody that specifically binds to a target antigen can also specifically bind to another antigen, e.g., an ortholog of the target antigen.

As used herein, the terms “treat,” “treating,” or the like, mean to alleviate symptoms, eliminate the causation of symptoms either on a temporary or permanent basis, or to prevent or slow the appearance of symptoms of the named disorder or condition.

As used herein, the term “subject in need thereof” means a human or non-human animal that exhibits one or more symptoms or indicia of allergy (e.g., food allergy), and/or who has been diagnosed with allergy to an allergen (e.g., a food allergen). The terms “subject” and “patient” are used interchangeably herein. In certain embodiments, the term “subject in need thereof” refers to a subject having an allergy to one or more of peanut, hazelnut, walnut, cashew, milk, egg/egg white, soy, wheat, sesame, cod, salmon, tuna, lobster, crab and/or shrimp. In some embodiments, a “subject in need thereof” refers to a subject with IgE-mediated allergy (e.g., IgE-mediated food allergy). In some embodiments, the term “subject in need thereof” includes a subject having an elevated level of one or more serum biomarkers of allergy, including but not limited to total IgE or allergen-specific IgE.

Although any methods and materials similar or equivalent to those described herein can be used in the practice of the disclosure, the typical methods and materials are now described. All publications mentioned herein are incorporated herein by reference in their entirety.

### Therapeutic Methods

In one aspect, provided herein are pharmaceutical compositions, combinations, and methods for treating a subject having an IgE-mediated allergy or preventing or reducing the severity of an allergic reaction in a subject having an IgE-mediated allergy, comprising administering to the subject an IL-4R antagonist, such as an anti-IL-4Rα antibody or antigen-binding fragment thereof, and a BCMA targeting agent, such as a bispecific antigen-binding molecule comprising a first antigen-binding domain that specifically binds BCMA and a second antigen-binding domain that specifically binds CD3.

In another aspect, provided herein are pharmaceutical compositions, combinations, and methods for reducing or eliminating serum total IgE in a subject in need thereof, comprising administering to the subject an interleukin-4 receptor (IL-4R) antagonist, such as an anti-IL-4Rα antibody or antigen-binding fragment thereof, and a B-cell maturation antigen (BCMA) targeting agent, such as a bispecific antigen-binding molecule comprising a first antigen-binding domain that specifically binds BCMA and a second antigen-binding domain that specifically binds CD3. In some embodiments, the subject is a subject having an IgE-mediated allergy. In some embodiments, total IgE is reduced by at least 20%, at least 30%, at least 40%, at least 50%, at least 60%, at least 70%, at least 80%, at least 90%, or more (e.g., after administration of 1 dose of the BCMA targeting agent, after administration of 2 doses of the BCMA targeting agent, after administration of 3 doses of the BCMA targeting agent, after administration of 4 doses of the BCMA targeting agent, or after administration of 5 doses of the BCMA targeting agent; or at day 1, day 15, day 29, day 36, day 43, day 50, day 57, day 64, day 71, day 85, day 113, day 141, day 169, day 197, day 210, day 239, day 267, day 295, day 323, or day 351 from the start of treatment; or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 30, week 32, week 34, week 36, week 38, week 40, week 42, week 44, week 46, week 48, week 50, or week 52 from the start of treatment). In some embodiments, total IgE is reduced to a level that is below the lower limit of quantitation. In some embodiments, total IgE is reduced to <2 kU/L.

In another aspect, provided herein are pharmaceutical compositions, combinations, and methods for reducing allergen-specific IgE in a subject in need thereof, comprising administering to the subject an interleukin-4 receptor (IL-4R) antagonist, such as an anti-IL-4Rα antibody or antigen-binding fragment thereof, and a B-cell maturation antigen (BCMA) targeting agent, such as a bispecific antigen-binding molecule comprising a first antigen-binding domain that specifically binds BCMA and a second antigen-binding domain that specifically binds CD3. In some embodiments, allergen-specific IgE is reduced by at least 20%, at least 30%, at least 40%, at least 50%, at least 60%, or more (e.g., after administration of 1 dose of the BCMA targeting agent, after administration of 2 doses of the BCMA targeting agent, after administration of 3 doses of the BCMA targeting agent, after administration of 4 doses of the BCMA targeting agent, or after administration of 5 doses of the BCMA targeting agent; or at day 1, day 15, day 29, day 36, day 43, day 50, day 57, day 64, day 71, day 85, day 113, day 141, day 169, day 197, day 210, day 239, day 267, day 295, day 323, or day 351 from the start of treatment; or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 30, week 32, week 34, week 36, week 38, week 40, week 42, week 44, week 46, week 48, week 50, or week 52 from the start of treatment). In some embodiments, allergen-specific IgE is reduced to <0.35 kU/L. In some embodiments, allergen-specific IgE is reduced to a level that is below the lower limit of quantitation.

In some embodiments, the subject to be treated has an IgE-mediated food allergy. In some embodiments, the subject has a severe IgE-mediated food allergy. In some embodiments, the subject has a history of anaphylaxis requiring epinephrine use, emergency room visit(s), and/or hospitalization for a food-induced allergic reaction, e.g., within 3 months, 6 months, 9 months, or 12 months of the start of treatment. In some embodiments, the subject to be treated has an IgE-mediated environmental allergy (e.g., to one or more perennial allergens).

In some embodiments, the subject has an allergy to peanut, hazelnut, walnut, cashew, milk, egg/egg white, soy, wheat, sesame, cod, salmon, tuna, lobster, crab, and/or shrimp. In some embodiments, the subject has multiple food allergies. In some embodiments, the subject has an allergy to peanut. In some embodiments, the subject has an allergy to tree nuts. In some embodiments, the subject has an allergy to sesame. In some embodiments, the subject has an allergy to cow's milk. In some embodiments, the subject has an allergy to wheat.

In some embodiments, the subject has a serum allergen-specific IgE ≥0.35 kU/L and/or a positive skin prick test (SPT) average diameter ≥4 mm wheal for at least one of the following food allergens: peanut, hazelnut, walnut, cashew, milk, egg, egg white, soy, wheat, sesame, cod, salmon, tuna, lobster, crab, and/or shrimp. In some embodiments, the subject has an allergy to one or more perennial allergens. In some embodiments, the subject has a serum allergen-specific IgE ≥0.35 kU/L and/or a positive skin prick test (SPT) average diameter ≥4 mm wheal for at least one of the following perennial allergens: ragweed, silver birch, white oak, Timothy grass, D. farina, dog dander, cat dander, and house dust mite.

In some embodiments, the subject has a serum total IgE of ≥150 kU/L prior to or at the start of treatment. In some embodiments, the subject has a baseline serum total IgE ≥200 kU/L, ≥250 kU/L, ≥300 kU/L, ≥350 kU/L, ≥400 kU/L, ≥450 kU/L, ≥500 kU/L, ≥600 KU/L, ≥700 kU/L, ≥800 KU/L, ≥900 kU/L, ≥1000 kU/L, ≥1250 kU/L, ≥1500 kU/L, ≥1750 kU/L, ≥2000 kU/L or higher prior to or at the start of treatment. In some embodiments, the subject has a baseline serum allergen-specific IgE ≥0.35 kU/L to one or more allergens, e.g., one or more food allergens (e.g., peanut, hazelnut, walnut, cashew, milk, egg/egg white, soy, wheat, sesame, cod, salmon, tuna, lobster, crab, or shrimp) and/or one or more environmental allergens. In some embodiments, the subject has a baseline serum allergen-specific IgE ≥0.35 kU/L to one or more allergens (e.g., one or more food allergens) and a baseline serum total IgE of ≥150 kU/L.

In some embodiments, a subject to be treated is an adult. In some embodiments, the subject is an adolescent. In some embodiments, the subject is ≥12 years of age. In some embodiments, the subject is a pediatric subject <18 years of age, e.g., <12 years of age, <10 years of age, <8 years of age, <6 years of age, <4 years of age, or <2 years of age.

In some embodiments, a subject to be treated is on an elemental diet, an elimination diet, or an empiric diet.

In some embodiments, a subject to be treated has, or has had, at least one comorbidity. In some embodiments, a subject to be treated has, or has had, a concomitant type 2 inflammatory condition. As used herein, a “type 2 inflammation condition” is a disease, disorder, or condition associated with a T helper 2 (TH2)-mediated immune response (Gandhi, et al., Nat Rev Drug Discov., 2016, 15 (1): 35-50). Non-limiting examples of type 2 inflammatory conditions include allergic bronchopulmonary aspergillosis (ABPA), allergic rhinitis, allergic fungal rhinosinusitis, aspirin hypersensitivity, asthma, atopic conjunctivitis, atopic dermatitis, bullous pemphigoid, chronic eosinophilic pneumonia (CEP), chronic obstructive pulmonary disease (COPD), chronic rhinosinusitis, chronic rhinosinusitis with nasal polyps (CRSwNP), chronic sinusitis, cystic fibrosis, eosinophilic esophagitis (EoE), eosinophilic duodenitis, eosinophilic gastritis, eosinophilic gastroenteritis, eosinophilic granulomatosis with polyangiitis (EGPA, formerly known as Churg-Strauss syndrome), exercise-induced bronchospasm, nonsteroidal anti-inflammatory drug (NSAID) hypersensitivity (e.g., NSAIDs Exacerbated Respiratory Disease, or NSAID-ERD), perennial allergic rhinitis (PAR), prurigo nodularis, unified airway disease, vasculitis, and venom hypersensitivity. In some embodiments, the subject has a concomitant atopic disease or condition selected from the group consisting of atopic dermatitis, asthma, chronic rhinosinusitis, allergic rhinitis, or allergic conjunctivitis. In some embodiments, the subject has atopic dermatitis. In some embodiments, the subject has eosinophilic esophagitis. In some embodiments, the subject does not have a concomitant atopic disease or condition.

In some embodiments, a subject to be treated is selected on the basis of exhibiting one or more inclusion criteria disclosed in Example 1. In some embodiments, a subject to be treated is further selected on the basis of not exhibiting one or more exclusion criteria disclosed in Example 1.

In some embodiments, treatment according to the methods disclosed herein reduces serum total IgE by at least 20%, at least 30%, at least 40%, at least 50%, at least 60%, at least 70%, at least 80%, at least 90%, or more. In some embodiments, total IgE is reduced at least 20%, at least 30%, at least 40%, at least 50%, at least 60%, at least 70%, at least 80%, or more after administration of 1 dose of the BCMA targeting agent. In some embodiments, total IgE is reduced at least 20%, at least 30%, at least 40%, at least 50%, at least 60%, at least 70%, at least 80%, or more after administration of 2 doses of the BCMA targeting agent. In some embodiments, total IgE is reduced at least 20%, at least 30%, at least 40%, at least 50%, at least 60%, at least 70%, at least 80%, or more after administration of 3 doses of the BCMA targeting agent. In some embodiments, total IgE is reduced at least 20%, at least 30%, at least 40%, at least 50%, at least 60%, at least 70%, at least 80%, or more after administration of 4 doses of the BCMA targeting agent. In some embodiments, total IgE is reduced at least 20%, at least 30%, at least 40%, at least 50%, at least 60%, at least 70%, at least 80%, or more or after administration of 6 doses of the BCMA targeting agent. In some embodiments, total IgE is reduced at least 20%, at least 30%, at least 40%, at least 50%, at least 60%, at least 70%, at least 80%, or more after administration of 7 doses of the BCMA targeting agent. In some embodiments, total IgE is reduced at least 20%, at least 30%, at least 40%, at least 50%, at least 60%, at least 70%, at least 80%, or more after the course of treatment with the BCMA targeting agent is completed. In some embodiments, serum total IgE is reduced to a level that is below the lower limit of quantitation. In some embodiments, serum total IgE is reduced to <2 kU/L. In some embodiments, reduction in total IgE is sustained for at least 1 week, at least 2 weeks, at least 3 weeks, at least 4 weeks, or longer after administration of the BCMA targeting agent or after administration of the last dose of the BCMA targeting agent. In some embodiments, reduction in total IgE is sustained for at least 1 month, at least 2 months, at least 3 months, at least 4 months, at least 5 months, at least 6 months, at least 7 months, at least 8 months, at least 9 months, at least 10 months, at least 11 months, at least 12 months, or longer after administration of the BCMA targeting agent or after administration of the last dose of the BCMA targeting agent. In some embodiments, reduction in total IgE is sustained for at least 1 year after administration of the BCMA targeting agent or after administration of the last dose of the BCMA targeting agent. In some embodiments, treatment according to the methods disclosed herein reduces serum total IgE by at least 20%, at least 30%, at least 40%, at least 50%, at least 60%, at least 70%, at least 80%, at least 90%, or more at day 8, day 15, day 29, day 36, day 43, day 50, day 57, day 64, day 71, day 85, day 113, day 141, day 169, day 197, day 210, day 239, day 267, day 295, day 323, or day 351 from the start of treatment, or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 30, week 32, week 34, week 36, week 38, week 40, week 42, week 44, week 46, week 48, week 50, or week 52 from the start of treatment.

In some embodiments, treatment according to the methods disclosed herein reduces allergen-specific IgE (e.g., IgE for one or more food allergens) by at least 20%, at least 30%, at least 40%, at least 50%, at least 60%, at least 70%, at least 80%, at least 90%, or more. In some embodiments, allergen-specific IgE is reduced at least 20%, at least 30%, at least 40%, at least 50%, at least 60%, at least 70%, at least 80%, or more after administration of 1 dose of the BCMA targeting agent. In some embodiments, allergen-specific IgE is reduced at least 20%, at least 30%, at least 40%, at least 50%, at least 60%, at least 70%, at least 80%, or more after administration of 2 doses of the BCMA targeting agent. In some embodiments, allergen-specific IgE is reduced at least 20%, at least 30%, at least 40%, at least 50%, at least 60%, at least 70%, at least 80%, or more after administration of 3 doses of the BCMA targeting agent. In some embodiments, allergen-specific IgE is reduced at least 20%, at least 30%, at least 40%, at least 50%, at least 60%, at least 70%, at least 80%, or more after administration of 4 doses of the BCMA targeting agent. In some embodiments, allergen-specific IgE is reduced at least 20%, at least 30%, at least 40%, at least 50%, at least 60%, at least 70%, at least 80%, or more or after administration of 6 doses of the BCMA targeting agent. In some embodiments, allergen-specific IgE is reduced at least 20%, at least 30%, at least 40%, at least 50%, at least 60%, at least 70%, at least 80%, or more after administration of 7 doses of the BCMA targeting agent. In some embodiments, allergen-specific IgE is reduced at least 20%, at least 30%, at least 40%, at least 50%, at least 60%, at least 70%, at least 80%, or more after the course of treatment with the BCMA targeting agent is completed. In some embodiments, allergen-specific IgE is reduced to <0.35 kU/L. In some embodiments, reduction in allergen-specific IgE is sustained for at least 1 week, at least 2 weeks, at least 3 weeks, at least 4 weeks, or longer after administration of the BCMA targeting agent or after administration of the last dose of the BCMA targeting agent. In some embodiments, reduction in allergen-specific IgE is sustained for at least 1 month, at least 2 months, at least 3 months, at least 4 months, at least 5 months, at least 6 months, at least 7 months, at least 8 months, at least 9 months, at least 10 months, at least 11 months, at least 12 months, or longer after administration of the BCMA targeting agent or after administration of the last dose of the BCMA targeting agent. In some embodiments, reduction in allergen-specific IgE is sustained for at least 1 year after administration of the BCMA targeting agent or after administration of the last dose of the BCMA targeting agent. In some embodiments, treatment according to the methods disclosed herein reduces allergen-specific IgE (e.g., IgE for one or more food allergens) by at least 20%, at least 30%, at least 40%, at least 50%, at least 60%, at least 70%, at least 80%, at least 90%, or more at day 8, day 15, day 29, day 36, day 43, day 50, day 57, day 64, day 71, day 85, day 113, day 141, day 169, day 197, day 210, day 239, day 267, day 295, day 323, or day 351 from the start of treatment, or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, week 25, week 26, week 27, week 28, week 30, week 32, week 34, week 36, week 38, week 40, week 42, week 44, week 46, week 48, week 50, or week 52 from the start of treatment.

### Interleukin-4 Receptor Antagonists

In some embodiments, the methods of the present disclosure comprise administering to a subject in need thereof (e.g., a subject having a food allergy) an interleukin-4 receptor (IL-4R) antagonist or a pharmaceutical composition comprising an IL-4R antagonist. As used herein, an “IL-4R antagonist” (also referred to herein as an “IL-4R inhibitor”, an “IL-4R blocker,” or an “IL-4Rα antagonist”) is any agent that binds to or interacts with IL-4Rα or an IL-4R ligand, and inhibits or attenuates the normal biological signaling function of a type 1 and/or a type 2 IL-4 receptor. Human IL-4Rα has the amino acid sequence of SEQ ID NO:11. A type 1 IL-4 receptor is a dimeric receptor comprising an IL-4Rα chain and a γc chain. A type 2 IL-4 receptor is a dimeric receptor comprising an IL-4Rα chain and an IL-13Rα1 chain. Type 1 IL-4 receptors interact with and are stimulated by IL-4, while type 2 IL-4 receptors interact with and are stimulated by both IL-4 and IL-13. Thus, the IL-4R antagonists that can be used in the methods of the present disclosure may function by blocking IL-4-mediated signaling, IL-13-mediated signaling, or both IL-4- and IL-13-mediated signaling. The IL-4R antagonists of the present disclosure may thus prevent the interaction of IL-4 and/or IL-13 with a type 1 or type 2 receptor.

Non-limiting examples of categories of IL-4R antagonists include small molecule IL-4R inhibitors, anti-IL-4R aptamers, peptide-based IL-4R inhibitors (e.g., “peptibody” molecules), “receptor-bodies” (e.g., engineered molecules comprising the ligand-binding domain of an IL-4R component), and antibodies or antigen-binding fragments of antibodies that specifically bind human IL-4Rα. As used herein, IL-4R antagonists also include antigen-binding proteins that specifically bind IL-4 and/or IL-13.

### Anti-IL-4Rα Antibodies and Antigen-Binding Fragments Thereof

In certain exemplary embodiments of the present disclosure, the IL-4R antagonist is an antibody that specifically binds IL-4Rα (also referred to herein as an “anti-IL-4Rα antibody” or “IL-4Rα antibody”) or an antigen-binding fragment thereof. In some embodiments, the anti-IL-4Ra antibody or antigen-binding fragment thereof is a human antibody or antigen-binding fragment thereof. In some embodiments, the anti-IL-4Rα antibody or antigen-binding fragment thereof is a recombinant antibody (e.g., a recombinant human antibody). In some embodiments, the anti-IL-4Rα antibody is a full antibody (e.g., an IgG antibody). In some embodiments, the full antibody is an IgG4 antibody. In some embodiments, the full antibody is an IgG1 antibody.

In some embodiments, the IL-4R antagonist is an anti-IL-4Rα antibody, or antigen-binding fragment thereof, comprising a heavy chain variable region (HCVR), light chain variable region (LCVR), and/or complementarity determining regions (CDRs) comprising any of the amino acid sequences of the anti-IL-4R antibodies as set forth in U.S. Pat. No. 7,608,693, incorporated by reference herein. In some embodiments, the IL-4R antagonist is an anti-IL-4Rα antibody or antigen-binding fragment thereof that comprises the heavy chain complementarity determining regions (HCDRs) of a heavy chain variable region (HCVR) comprising the amino acid sequence of SEQ ID NO:1 and the light chain complementarity determining regions (LCDRs) of a light chain variable region (LCVR) comprising the amino acid sequence of SEQ ID NO:2. In some embodiments, the IL-4R antagonist is an anti-IL-4Rα antibody or antigen-binding fragment thereof that comprises three HCDRs (HCDR1, HCDR2 and HCDR3) and three LCDRs (LCDR1, LCDR2 and LCDR3), wherein the HCDR1 comprises the amino acid sequence GFTFRDYA (SEQ ID NO:3), the HCDR2 comprises the amino acid sequence ISGSGGNT (SEQ ID NO:4), the HCDR3 comprises the amino acid sequence AKDRLSITIRPRYYGLDV (SEQ ID NO:5), the LCDR1 comprises the amino acid sequence QSLLYSIGYNY (SEQ ID NO:6), the LCDR2 comprises the amino acid sequence LGS (SEQ ID NO:7), and the LCDR3 comprises the amino acid sequence MQALQTPYT (SEQ ID NO:8).

In some embodiments, the anti-IL-4R antibody or antigen-binding fragment thereof comprises an HCDR1 comprising the amino acid sequence GFTFRDYA (SEQ ID NO:3), an HCDR2 comprising the amino acid sequence ISGSGGNT (SEQ ID NO:4), an HCDR3 comprising the amino acid sequence AKDRLSITIRPRYYGLDV (SEQ ID NO:5), an LCDR1 comprising the amino acid sequence QSLLYSIGYNY (SEQ ID NO:6), an LCDR2 comprising the amino acid sequence LGS (SEQ ID NO:7), and an LCDR3 comprising the amino acid sequence MQALQTPYT (SEQ ID NO:8), and further comprises an HCVR having at least 85% sequence identity (e.g., at least 90%, 91%, 92%, 93%, 94%, 95%, 96%, 97%, 98%, or 99% sequence identity) to the amino acid sequence of SEQ ID NO:1 and an LCVR having at least 85% sequence identity (e.g., at least 90%, 91%, 92%, 93%, 94%, 95%, 96%, 97%, 98%, or 99% sequence identity) to the amino acid sequence of SEQ ID NO:2. In some embodiments, the anti-IL-4R antibody or antigen-binding fragment thereof comprises an HCVR comprising SEQ ID NO:1 and an LCVR comprising SEQ ID NO:2.

In some embodiments, the anti-IL-4R antibody comprises a heavy chain comprising the amino acid sequence of SEQ ID NO:9. In some embodiments, the anti-IL-4R antibody comprises a heavy chain comprising the amino acid sequence of SEQ ID NO:387. In some embodiments, the anti-IL-4R antibody comprises a light chain comprising the amino acid sequence of SEQ ID NO:10.

An exemplary antibody comprising a heavy chain comprising the amino acid sequence of SEQ ID NO:9 or SEQ ID NO:387 and a light chain comprising the amino acid sequence of SEQ ID NO:10 is the fully human anti-IL-4R antibody known as dupilumab. According to certain exemplary embodiments, the methods of the present disclosure comprise the use of dupilumab. As used herein, “dupilumab” also includes bioequivalents of dupilumab. The term “bioequivalent,” as used herein with reference to dupilumab, refers to anti-IL-4R antibodies or IL-4R-binding proteins or fragments thereof that are pharmaceutical equivalents or pharmaceutical alternatives whose rate and/or extent of absorption do not show a significant difference with that of dupilumab when administered at the same molar dose under similar experimental conditions, either single dose or multiple doses. In some embodiments, the term refers to antigen-binding proteins that bind to IL-4R which do not have clinically meaningful differences with dupilumab in their safety, purity, and/or potency.

Other anti-IL-4Rα antibodies that can be used in the context of the methods of the present disclosure include, e.g., the antibody referred to and known in the art as AMG317 (Corren, et al., 2010, Am J Respir Crit Care Med., 181 (8): 788-796), APG808, BA2101, comekibart, GR1802, manfidokimab, LQ036, MEDI2045, MEDI9314, NS-402, QX-005N, rademikibart, SHR-1819, stapokibart, TQH-2722, 611 (Sunshine Guojian), or any of the anti-IL-4Ra antibodies as set forth in U.S. Pat. Nos. 7,186,809, 7,605,237, 7,638,606, 8,092,804, 8,679,487, 8,877,189, 11,667,717, 11,725,057, 10,774,141, 11,891,441, 11,897,960, 11,939,387, International Patent Publication Nos. WO2020/096381, WO 2020/135471, WO 2020/135710, WO 2020/182197, WO2020/239134, WO 2021/213329, WO2022/052974, WO2022/136669, WO2022/136675, or WO2024/173847, or US Patent Publication Nos. 2021/0238294, 2022/0073631, 2022/0162328, 2022/0411519, 2023/0105029, or 2023/0295312, the contents of each of which are incorporated by reference herein.

In some embodiments, an anti-IL-4Rα antibody or antigen-binding fragment thereof for use in the methods of the present disclosure comprises one or more CDR, HCVR, and/or LCVR sequences set forth in Table 1 below.

In some embodiments, an anti-IL-4Rα antibody comprises (i) an HCVR comprising the amino acid sequence of SEQ ID NO:32 (SCB-VH-59), SEQ ID NO:33 (SCB-VH-60), SEQ ID NO:34 (SCB-VH-61), SEQ ID NO:35 (SCB-VH-62), SEQ ID NO:36 (SCB-VH-63), SEQ ID NO:37 (SCB-VH-64), SEQ ID NO:38 (SCB-VH-65), SEQ ID NO:39 (SCB-VH-66), SEQ ID NO:40 (SCB-VH-67), SEQ ID NO:41 (SCB-VH-68), SEQ ID NO:42 (SCB-VH-69), SEQ ID NO:43 (SCB-VH-70), SEQ ID NO:44 (SCB-VH-71), SEQ ID NO:45 (SCB-VH-72), SEQ ID NO:46 (SCB-VH-73), SEQ ID NO:47 (SCB-VH-74), SEQ ID NO:48 (SCB-VH-75), SEQ ID NO:49 (SCB-VH-76), SEQ ID NO:50 (SCB-VH-77), SEQ ID NO:51 (SCB-VH-78), SEQ ID NO:52 (SCB-VH-79), SEQ ID NO:53 (SCB-VH-80), SEQ ID NO:54 (SCB-VH-81), SEQ ID NO:55 (SCB-VH-82), SEQ ID NO:56 (SCB-VH-83), SEQ ID NO:57 (SCB-VH-84), SEQ ID NO:58 (SCB-VH-85), SEQ ID NO:59 (SCB-VH-86), SEQ ID NO:60 (SCB-VH-87), SEQ ID NO:61 (SCB-VH-88), SEQ ID NO:62 (SCB-VH-89), SEQ ID NO:63 (SCB-VH-90), SEQ ID NO:64 (SCB-VH-91), SEQ ID NO:65 (SCB-VH-92), or SEQ ID NO:66 (SCB-VH-93); and (ii) an LCVR comprising the amino acid sequence of SEQ ID NO:12 (SCB-VL-39), SEQ ID NO:13 (SCB-VL-40), SEQ ID NO:14 (SCB-VL-41), SEQ ID NO:15 (SCB-VL-42), SEQ ID NO:16 (SCB-VL-43), SEQ ID NO:17 (SCB-VL-44), SEQ ID NO:18 (SCB-VL-45), SEQ ID NO:19 (SCB-VL-46), SEQ ID NO:20 (SCB-VL-47), SEQ ID NO:21 (SCB-VL-48), SEQ ID NO:22 (SCB-VL-49), SEQ ID NO:23 (SCB-VL-50), SEQ ID NO:24 (SCB-VL-51), SEQ ID NO:25 (SCB-VL-52), SEQ ID NO:26 (SCB-VL-53), SEQ ID NO:27 (SCB-VL-54), SEQ ID NO:28 (SCB-VL-55), SEQ ID NO:29 (SCB-VL-56), SEQ ID NO:30 (SCB-VL-57), or SEQ ID NO:31 (SCB-VL-58). In some embodiments, the anti-IL-4Rα antibody comprises an HCVR comprising the amino acid sequence of SEQ ID NO:64 (SCB-VH-91) and an LCVR comprising the amino acid sequence of SEQ ID NO:17 (SCB-VL-44), SEQ ID NO:27 (SCB-VL-54), or SEQ ID NO:28 (SCB-VL-55). In some embodiments, the anti-IL-4Rα antibody comprises an LCVR/HCVR sequence pair selected from the group consisting of SCB-VL-39/SCB-VH-92; SCB-VL-40/SCB-VH-92; SCB-VL-41/SCB-VH-92; SCB-VL-42/SCB-VH-92; SCB-VL-43/SCB-VH-92; SCB-VL-44/SCB-VH-92; SCB-VL-44/SCB-VH-62; SCB-VL-44/SCB-VH-68; SCB-VL-44/SCB-VH-72; SCB-VL-44/SCB-VH-82; SCB-VL-44/SCB-VH-85; SCB-VL-44/SCB-VH-91; SCB-VL-44/SCB-VH-93; SCB-VL-45/SCB-VH-92; SCB-VL-46/SCB-VH-92; SCB-VL-47/SCB-VH-92; SCB-VL-48/SCB-VH-92; SCB-VL-49/SCB-VH-92; SCB-VL-50/SCB-VH-92; SCB-VL-51/SCB-VH-92; SCB-VL-51/SCB-VH-93; SCB-VL-52/SCB-VH-92; SCB-VL-52/SCB-VH-62; SCB-VL-52/SCB-VH-91; SCB-VL-53/SCB-VH-92; SCB-VL-54/SCB-VH-92; SCB-VL-54/SCB-VH-62; SCB-VL-54/SCB-VH-68; SCB-VL-54/SCB-VH-72; SCB-VL-54/SCB-VH-82; SCB-VL-54/SCB-VH-85; SCB-VL-54/SCB-VH-91; SCB-VL-55/SCB-VH-92; SCB-VL-55/SCB-VH-62; SCB-VL-55/SCB-VH-68; SCB-VL-55/SCB-VH-72; SCB-VL-55/SCB-VH-82; SCB-VL-55/SCB-VH-85; SCB-VL-55/SCB-VH-91; SCB-VL-56/SCB-VH-92; SCB-VL-57/SCB-VH-92; SCB-VL-57/SCB-VH-93; SCB-VL-57/SCB-VH-59; SCB-VL-57/SCB-VH-60; SCB-VL-57/SCB-VH-61; SCB-VL-57/SCB-VH-62; SCB-VL-57/SCB-VH-63; SCB-VL-57/SCB-VH-64; SCB-VL-57/SCB-VH-65; SCB-VL-57/SCB-VH-66; SCB-VL-57/SCB-VH-67; SCB-VL-57/SCB-VH-68; SCB-VL-57/SCB-VH-69; SCB-VL-57/SCB-VH-70; SCB-VL-57/SCB-VH-71; SCB-VL-57/SCB-VH-72; SCB-VL-57/SCB-VH-73; SCB-VL-57/SCB-VH-74; SCB-VL-57/SCB-VH-75; SCB-VL-57/SCB-VH-76; SCB-VL-57/SCB-VH-77; SCB-VL-57/SCB-VH-78; SCB-VL-57/SCB-VH-79; SCB-VL-57/SCB-VH-80; SCB-VL-57/SCB-VH-81; SCB-VL-57/SCB-VH-82; SCB-VL-57/SCB-VH-83; SCB-VL-57/SCB-VH-84; SCB-VL-57/SCB-VH-85; SCB-VL-57/SCB-VH-86; SCB-VL-57/SCB-VH-87; SCB-VL-57/SCB-VH-88; SCB-VL-57/SCB-VH-89; SCB-VL-57/SCB-VH-90; SCB-VL-57/SCB-VH-91; SCB-VL-58/SCB-VH-91; SCB-VL-58/SCB-VH-92; and SCB-VL-58/SCB-VH-93.

In some embodiments, an anti-IL-4Rα antibody comprises an amino acid sequence pair selected from the group consisting of: SEQ ID NOs:67/68 (MEDI-1-VH/MEDI-1-VL); SEQ ID NOs:69/70 (MEDI-2-VH/MEDI-2-VL); SEQ ID NOS: 71/72 (MEDI-3-VH/MEDI-3-VL); SEQ ID NOs:73/74 (MEDI-4-VH/MEDI-4-VL); SEQ ID NOs:75/76 (MEDI-5-VH/MEDI-5-VL); SEQ ID NOs:77/78 (MEDI-6-VH/MEDI-6/VL); SEQ ID NOs:79/80 (MEDI-7-VH/MEDI-7-VL); SEQ ID NOs:81/82 (MEDI-8-VH/MEDI-8-VL); SEQ ID NOs:83/84 (MEDI-9-VH/MEDI-9-VL); SEQ ID NOs:85/86 (MEDI-10-VH/MEDI-10-VL); SEQ ID NOs:87/88 (MEDI-11-VH/MEDI-11/VL); SEQ ID NOs:89/90 (MEDI-12-VH/MEDI-12-VL); SEQ ID NOs:91/92 (MEDI-13-VH/MEDI-13-VL); SEQ ID NOs:93/94 (MEDI-14-VH/MEDI-14-VL); SEQ ID NOs:95/96 (MEDI-15-VH/MEDI-15-VL); SEQ ID NOs:97/98 (MEDI-16-VH/MEDI-16/VL); SEQ ID NOs:99/100 (MEDI-17-VH/MEDI-17-VL); SEQ ID NOs:101/102 (MEDI-18-VH/MEDI-18-VL); SEQ ID NOs:103/104 (MEDI-19-VH/MEDI-19-VL); SEQ ID NOs:105/106 (MEDI-20-VH/MEDI-20-VL); SEQ ID NOs:107/108 (MEDI-21-VH/MEDI-21-VL); SEQ ID NOs:109/110 (MEDI-22-VH/MEDI-22-VL); SEQ ID NOs:111/112 (MEDI-23-VH/MEDI-23-VL); SEQ ID NOs:113/114 (MEDI-24-VH/MEDI-24-VL); SEQ ID NOs:115/116 (MEDI-25-VH/MEDI-25-VL); SEQ ID NOs:117/118 (MEDI-26-VH/MEDI-26-VL); SEQ ID NOs:119/120 (MEDI-27-VH/MEDI-27-VL); SEQ ID NOs:121/122 (MEDI-28-VH/MEDI-28-VL); SEQ ID NOs:123/124 (MEDI-29-VH/MEDI-29-VL); SEQ ID NOs:125/126 (MEDI-30-VH/MEDI-30-VL); SEQ ID NOs:127/128 (MEDI-31-VH/MEDI-31-VL); SEQ ID NOs:129/130 (MEDI-32-VH/MEDI-32-VL); SEQ ID NOs:131/132 (MEDI-33-VH/MEDI-33-VL); SEQ ID NOs:133/134 (MEDI-34-VH/MEDI-34-VL); SEQ ID NOs:135/136 (MEDI-35-VH/MEDI-35-VL); SEQ ID NOs:137/138 (MEDI-36-VH/MEDI-36-VL); SEQ ID NOs:139/140 (MEDI-37-VH/MEDI-37-VL); SEQ ID NOs:141/142 (MEDI-38-VH/MEDI-38-VL); SEQ ID NOs:143/144 (MEDI-39-VH/MEDI-39-VL); SEQ ID NOs:145/146 (MEDI-40-VH/MEDI-40-VL); SEQ ID NOs:147/148 (MEDI-41-VH/MEDI-41-VL); SEQ ID NOs:149/150 (MEDI-42-VH/MEDI-42-VL); and SEQ ID NOs:151/152 (MEDI-37GL-VH/MEDI-37GL-VL).

In some embodiments, an anti-IL-4Rα antibody comprises (i) an HCVR comprising the amino acid sequence of SEQ ID NO:153 (AJOU-1-VH), SEQ ID NO:154 (AJOU-2-VH), SEQ ID NO:155 (AJOU-3-VH), SEQ ID NO:156 (AJOU-4-VH), SEQ ID NO:157 (AJOU-5-VH), SEQ ID NO:158 (AJOU-6-VH), SEQ ID NO:159 (AJOU-7-VH), SEQ ID NO:160 (AJOU-8-VH), SEQ ID NO:161 (AJOU-9-VH), SEQ ID NO:162 (AJOU-10-VH), SEQ ID NO:163 (AJOU-69-VH), SEQ ID NO:164 (AJOU-70-VH), SEQ ID NO:165 (AJOU-71-VH), SEQ ID NO:166 (AJOU-72-VH), or SEQ ID NO:167 (AJOU-83-VH); and (ii) an LCVR comprising the amino acid sequence of SEQ ID NO:168 (AJOU-33-VL), SEQ ID NO:169 (AJOU-34-VL), SEQ ID NO:170 (AJOU-35-VL), SEQ ID NO:171 (AJOU-36-VL), SEQ ID NO:172 (AJOU-37-VL), SEQ ID NO:173 (AJOU-38-VL), SEQ ID NO:174 (AJOU-39-VL), SEQ ID NO:175 (AJOU-40-VL), SEQ ID NO:176 (AJOU-41-VL), SEQ ID NO:177 (AJOU-42-VL), SEQ ID NO:178 (AJOU-77-VL), SEQ ID NO:179 (AJOU-78-VL), SEQ ID NO:180 (AJOU-79-VL), SEQ ID NO:181 (AJOU-80-VL), SEQ ID NO:182 (AJOU-86-VL), SEQ ID NO:183 (AJOU-87-VL), SEQ ID NO:184 (AJOU-88-VL), SEQ ID NO:185 (AJOU-89-VL), SEQ ID NO:186 (AJOU-90-VL), or SEQ ID NO:187 (AJOU-91-VL). In some embodiments, the anti-IL-4Rα antibody comprises the HCVR/LCVR sequence pair of AJOU-83-VH/AJOU-90-VL.

In some embodiments, an anti-IL-4Rα antibody comprises (i) an HCVR comprising the amino acid sequence of SEQ ID NO:188 (REGN-VH-3), SEQ ID NO:189 (REGN-VH-19), SEQ ID NO:190 (REGN-VH-35), SEQ ID NO:191 (REGN-VH-51), SEQ ID NO:192 (REGN-VH-67), SEQ ID NO:193 (REGN-VH-83), SEQ ID NO:194 (REGN-VH-99), SEQ ID NO:195 (REGN-VH-115), SEQ ID NO:196 (REGN-VH-147), or SEQ ID NO:197 (REGN-VH-163); and (ii) an LCVR comprising the amino acid sequence of SEQ ID NO:198 (REGN-VL-11), SEQ ID NO:199 (REGN-VL-27), SEQ ID NO:200 (REGN-VL-43), SEQ ID NO:201 (REGN-VL-59), SEQ ID NO:202 (REGN-VL-75), SEQ ID NO:203 (REGN-VL-91), SEQ ID NO:204 (REGN-VL-107), SEQ ID NO:205 (REGN-VL-123), SEQ ID NO:206 (REGN-VL-155), or SEQ ID NO:207 (REGN-VL-171). In some embodiments, the anti-IL-4Rα antibody comprises an HCVR/LCVR sequence pair selected from the group consisting of REGN-VH-3/REGN-VL-11; REGN-VH-19/REGN-VL-27; REGN-VH-35/REGN-VL-43; REGN-VH-51/REGN-VL-59; REGN-VH-67/REGN-VL-75; REGN-VH-83/REGN-VL-91; REGN-VH-99/REGN-VL-107; REGN-VH-115/REGN-VL-123; REGN-VH-147/REGN-VL-155; and REGN-VH-163/REGN-VL-171.

In some embodiments, an anti-IL-4Rα antibody comprises (i) an HCVR comprising the amino acid sequence of SEQ ID NO:208 (STSA-C27-VH), SEQ ID NO:209 (STSA-C27-6-33-VH), SEQ ID NO:210 (STSA-C27-7-33-VH), SEQ ID NO:211 (STSA-C27-24-56-VH), SEQ ID NO:212 (STSA-C27-47-56-VH), SEQ ID NO:213 (STSA-C27-33-33-VH), SEQ ID NO:214 (STSA-C27-56-56-VH), SEQ ID NO:215 (STSA-C27-78-78-VH), SEQ ID NO:216 (STSA-C27-82-58-VH), SEQ ID NO:217 (STSA-C27-54-54-VH), SEQ ID NO:218 (STSA-C27-36-36-VH), SEQ ID NO:219 (STSA-C27-53-53-VH), SEQ ID NO:220 (STSA-C27-67-67-VH), SEQ ID NO:221 (STSA-C27-55-55-VH), SEQ ID NO:222 (STSA-C27-59-59-VH), SEQ ID NO:223 (STSA-C27-58-58-VH), SEQ ID NO:224 (STSA-C27-52-52-VH), or SEQ ID NO:225 (STSA-C27-Y2-Y2-VH); and (ii) an LCVR comprising the amino acid sequence of SEQ ID NO:226 (STSA-C27-VL), SEQ ID NO:227 (STSA-C27-6-33-VL), SEQ ID NO:228 (STSA-C27-7-33-VL), SEQ ID NO:229 (STSA-C27-24-56-VL), SEQ ID NO:230 (STSA-C27-47-56-VL), SEQ ID NO:231 (STSA-C27-33-33-VL), SEQ ID NO:232 (STSA-C27-56-56-VL), SEQ ID NO:233 (STSA-C27-78-78-VL), SEQ ID NO:234 (STSA-C27-82-58-VL), SEQ ID NO:235 (STSA-C27-54-54-VL), SEQ ID NO:236 (STSA-C27-36-36-VL), SEQ ID NO:237 (STSA-C27-53-53-VL), SEQ ID NO:238 (STSA-C27-67-67-VL), SEQ ID NO:239 (STSA-C27-55-55-VL), SEQ ID NO:240 (STSA-C27-59-59-VL), SEQ ID NO:241 (STSA-C27-58-58-VL), SEQ ID NO:242 (STSA-C27-52-52-VL), or SEQ ID NO:243 (STSA-C27-Y2-Y2-VL). In some embodiments, the anti-IL-4Rα antibody comprises an HCVR/LCVR sequence pair selected from the group consisting of SEQ ID NOs:208/226 (STSA-C27), SEQ ID NOs:209/227 (STSA-C27-6-33), SEQ ID NOs:210/228 (STSA-C27-7-33), SEQ ID NOs:211/229 (STSA-C27-24-56), SEQ ID NOs:212/230 (STSA-C27-47-56), SEQ ID NOs:213/231 (STSA-C27-33-33), SEQ ID NOs:214/232 (STSA-C27-56-56), SEQ ID NOs:215/233 (STSA-C27-78-78), SEQ ID NOs:216/234 (STSA-C27-82-58), SEQ ID NOs:217/235 (STSA-C27-54-54), SEQ ID NOs:218/236 (STSA-C27-36-36), SEQ ID NOs:219/237 (STSA-C27-53-53), SEQ ID NOs:220/238 (STSA-C27-67-67), SEQ ID NOs:221/239 (STSA-C27-55-55), SEQ ID NOs:222/240 (STSA-C27-59-59), SEQ ID NOs:223/241 (STSA-C27-58-58), SEQ ID NOs:224/242 (STSA-C27-52-52), or SEQ ID NOs:225/243 (STSA-C27-Y2-Y2).

In some embodiments, an anti-IL-4Rα antibody comprises (i) an HCVR comprising the amino acid sequence of SEQ ID NO:244 (Y0188-1 VH), SEQ ID NO:245 (Y0188-2 VH), SEQ ID NO:246 (Y0188-3 VH), SEQ ID NO:247 (Y0188-4 VH), SEQ ID NO:248 (Y0188-6 VH), SEQ ID NO:249 (Y0188-8 VH), SEQ ID NO:250 (Y0188-9 VH), SEQ ID NO:251 (Y0188-10 VH), SEQ ID NO:252 (Y0188-14 VH), SEQ ID NO:253 (HV3-15-14 VH), SEQ ID NO:254 (HV3-48-14 VH), SEQ ID NO:255 (HV3-73*2-14 VH), SEQ ID NO:256 (HV3-72-14 VH), SEQ ID NO:257 (Y01-14 VH), SEQ ID NO:258 (162-14 VH), or SEQ ID NO:259 (VH73-14 VH); and (ii) an LCVR comprising the amino acid sequence of SEQ ID NO:260 (Y0188-1 VL), SEQ ID NO:261 (Y0188-2 VL), SEQ ID NO:262 (Y0188-3 VL), SEQ ID NO:263 (Y0188-4 VL), SEQ ID NO:264 (Y0188-6 VL), SEQ ID NO:265 (Y0188-8 VL), SEQ ID NO:266 (Y0188-9 VL), SEQ ID NO:267 (Y0188-10 VL), SEQ ID NO:268 (Y0188-14 VL), SEQ ID NO:269 (Y01-14 VL), SEQ ID NO:270 (164-14 VL), SEQ ID NO:271 (KV4-14 VL), SEQ ID NO:272 (KV1-27-14 VL), SEQ ID NO:273 (KV1-9-14 VL), SEQ ID NO:274 (KV1-NL1-14 VL), or SEQ ID NO:275 (KV1D-43-14 VL). In some embodiments, the anti-IL-4Rα antibody comprises an HCVR/LCVR sequence pair selected from the group consisting of SEQ ID NOs:244/260 (Y0188-1), SEQ ID NOs:245/261 (Y0188-2), SEQ ID NOs:246/262 (Y0188-3), SEQ ID NOs:247/263 (Y0188-4), SEQ ID NOs:248/264 (Y0188-6), SEQ ID NOs:249/265 (Y0188-8), SEQ ID NOs:250/266 (Y0188-9), SEQ ID NOs:251/267 (Y0188-10), SEQ ID NOs:252/268 (Y0188-14), SEQ ID NOs:257/269 (Y01-14), and SEQ ID NOs:258/270 (162-14).

In some embodiments, an anti-IL-4Rα antibody comprises (i) an HCVR comprising the amino acid sequence of SEQ ID NO:276 (1A6 VH), SEQ ID NO:278 (1D8 VH), SEQ ID NO:280 (1H9 VH), SEQ ID NO:282 (2H1 VH), SEQ ID NO:284 (2F8 VH), SEQ ID NO:286 (9B4 VH), SEQ ID NO:288 (9E7 VH), SEQ ID NO:290 (24G10 VH), SEQ ID NO:292 (25D6 VH), SEQ ID NO:294 (25G9 VH), SEQ ID NO:296 (35A7-1 VH), SEQ ID NO:298 (31B9 VH), SEQ ID NO:300 (34A2 VH), SEQ ID NO:302 (34H11 VH), SEQ ID NO:304 (35D5 VH), SEQ ID NO:306 (35A7-2 VH), SEQ ID NO:308 (36F4 VH), SEQ ID NO:310 (VH1021), SEQ ID NO:311 (VH1022), SEQ ID NO:312 (VH1023), SEQ ID NO:313 (VH1024), SEQ ID NO:314 (VH1025), SEQ ID NO:315 (VH1026), SEQ ID NO:316 (VH1027), or SEQ ID NO:317 (VH1028); and (ii) an LCVR comprising the amino acid sequence of SEQ ID NO:277 (1A6 VL), SEQ ID NO:279 (1D8 VL), SEQ ID NO:281 (1H9 VL), SEQ ID NO:283 (2H1 VL), SEQ ID NO:285 (2F8 VL), SEQ ID NO:287 (9B4 VL), SEQ ID NO:289 (9E7 VL), SEQ ID NO:291 (24G10 VL), SEQ ID NO:293 (25D6 VL), SEQ ID NO:295 (25G9 VL), SEQ ID NO:297 (35A7-1 VL), SEQ ID NO:299 (31B9 VL), SEQ ID NO:301 (34A2 VL), SEQ ID NO:303 (34H11 VL), SEQ ID NO:305 (35D5 VL), SEQ ID NO:307 (35A7-2 VL), SEQ ID NO:309 (36F4 VL), SEQ ID NO:318 (VL1011), SEQ ID NO:319 (VL012), SEQ ID NO:320 (VL1013), or SEQ ID NO:321 (VL1014). In some embodiments, the anti-IL-4Rα antibody comprises an HCVR/LCVR sequence pair selected from the group consisting of 1A6 VH/1A6 VL; 1D8 VH/1D8 VL; 1H9 VH/1H9 VL; 2H1 VH/2H1 VL; 2F8 VH/2F8 VL; 9B4 VH/9B4 VL; 9E7 VH/9E7 VL; 24G10 VH/24G10 VL; 25D6 VH/25D6 VL; 25G9 VH/25G9 VL; 35A7-1 VH/35A7-1 VL; 31B9 VH/31B9 VL; 34A2 VH/34A2 VL; 34H11 VH/34H11 VL; 35D5 VH/35D5 VL; 35A7-2 VH/35A7-2 VL; and 36F4 VH/36F4 VL.

In some embodiments, an anti-IL-4Rα antibody comprises an amino acid sequence pair selected from the group consisting of: SEQ ID NOs:322/323 (13E5 VH/13E5 VL), SEQ ID NOs:324/325 (13E5 H1 VH/13E5 L1 VL); and SEQ ID NOS: 326/327 (13E5 H4 VH/13E5 L4 VL).

In some embodiments, an anti-IL-4Rα antibody comprises an amino acid sequence pair selected from the group consisting of: SEQ ID NOs:328/329 (PD2-31 VH/PD2-31 VL) and SEQ ID NOs:330/331 (HZD82-12 VH/HZD82-12 VL).

In some embodiments, an anti-IL-4Rα antibody comprises an amino acid sequence pair selected from the group consisting of: SEQ ID NOs:332/333 (25G7 VH/25G7 VL), SEQ ID NOs:334/335 (hu25G7-VH/hu25G7-A VL), and SEQ ID NOs:334/336 (hu25G7-VH/hu25G7-B VL).

In some embodiments, an anti-IL-4Rα antibody comprises an amino acid sequence pair selected from the group consisting of: SEQ ID NOs:337/338 (ab 136 VH/ab 136 VL); and SEQ ID NOs:339/340 (136-Hu VH/136-Hu VL).

In some embodiments, an anti-IL-4Rα antibody comprises (i) an HCVR comprising the amino acid sequence of SEQ ID NO:341 (S1E6 VH); and (ii) an LCVR comprising the amino acid sequence of SEQ ID NO:342 (L18D7 VL), SEQ ID NO:343 (L28G5 VL), SEQ ID NO:344 (L28F8 VL), SEQ ID NO:345 (L28C9 VL), SEQ ID NO:346 (L10B2 VL), or SEQ ID NO:347 (L10C2 VL).

In some embodiments, an anti-IL-4Rα antibody comprises (i) an HCVR comprising the amino acid sequence of SEQ ID NO:348 (4-2 VH) or SEQ ID NO:350 (4-2-Humanized VH); and (ii) an LCVR comprising the amino acid sequence of SEQ ID NO:349 (4-2 VL) or SEQ ID NO:351 (4-2-Humanized VL). In some embodiments, an anti-IL-4Rα antibody comprises an amino acid sequence pair selected from the group consisting of: SEQ ID NOs:348/349 and SEQ ID NOs:350/351.

In some embodiments, an anti-IL-4Rα antibody comprises the amino acid sequence pair of SEQ ID NOs:352/353 (BA167 VH/BA167 VL).

In some embodiments, an anti-IL-4Rα antibody comprises the amino acid sequence pair of SEQ ID NOs:354/355 (huC2C1A1A1 VH/huB8G11F2B7G5E8 VL), SEQ ID NOs:356/355 (huB8G11F2B7G5E8-V11 VH/huB8G11F2B7G5E8 VL), SEQ ID NOs:357/355 (huB8G11F2B7G5E8-V14 VH/huB8G11F2B7G5E8 VL), SEQ ID NOs:358/359 (mB8D10G7G6E4 VH/mB8D10G7G6E4 VL), SEQ ID NOs:360/361 (mB9A7C9A4H5 VH/mB9A7C9A4H5 VL), SEQ ID NOs:362/363 (mB9D1D11F8D8 VH/mB9D1D11F8D8 VL), or SEQ ID NOs:364/365 (mB1D2F7D3B5 VH/mB1D2F7D3B5 VL).

### BCMA Targeting Agents

In some embodiments, the methods of the present disclosure comprise administering to a subject in need thereof (e.g., a subject having a food allergy) a B-cell maturation antigen (BCMA) targeting agent. As used herein, the term “BCMA targeting agent” refers to any molecule capable of binding specifically to BCMA expressed on the surface of a cell in a subject. BCMA is expressed in B-cell lineage cells, particularly in the interfollicular region of the germinal center as well as on plasmablasts and differentiated plasma cells. BCMA is selectively induced during plasma cell differentiation and is required for optimal survival of long-lived plasma cells in the bone marrow. Thus, in some embodiments, a BCMA targeting agent binds to BCMA expressed on the surface of plasma cells and mediates plasma cell killing or ablation.

In some embodiments, a BCMA targeting agent is an antibody that specifically binds BCMA. In some embodiments, a BCMA targeting agent comprises a binding moiety that binds to BCMA (an antigen-binding moiety or antigen-binding fragment thereof) and a moiety that facilitates killing of a cell expressing BCMA. In certain embodiments, the BCMA-binding moiety is an antibody or antigen-binding fragment thereof that binds specifically to BCMA. Such a BCMA-binding moiety is linked (e.g., covalently bound) to a moiety that facilitates killing or destruction of the targeted cell (e.g., plasma cell). The moiety that facilitates targeted killing of the cell may be a molecule that directly kills the targeted cell (e.g., a cytotoxic agent) or may be a protein or fragment thereof that mediates killing of the targeted cell by an immune cell, e.g., a T-cell. In the context of the present disclosure, the term “BCMA targeting agent” includes, but is not limited to, anti-BCMA antibodies that are conjugated to a therapeutic agent such as a cytotoxic drug (“BCMA ADC” or “anti-BCMA ADC”), chimeric antigenic receptors (CARs) that bind specifically to BCMA, (“BCMA CAR” or “anti-BCMA CAR”) and anti-BCMA x anti-CD3 bispecific antibodies.

In some embodiments, the BCMA targeting agent is an antibody-drug conjugate (ADC) comprising an anti-BCMA antibody and a cytotoxic drug. In some embodiments, the anti-BCMA antibody or antigen-binding fragment thereof and the cytotoxic agent are covalently attached via a linker. In general terms, the ADCs comprise: A-[L-P]y, in which A is an antigen-binding molecule, e.g., an anti-BCMA antibody, or a fragment thereof, L is a linker, P is the payload or therapeutic moiety (e.g., cytotoxic agent), and y is an integer from 1 to 30. Examples of suitable cytotoxic agents and chemotherapeutic agents for forming ADCs are known in the art. Non-limiting examples of suitable cytotoxic agents that can be conjugated to anti-BCMA antibodies for use in the disclosed methods are auristatin such as monomethyl auristatin E (MMAE) or monomethyl auristatin F (MMAF), a tubulysin such as TUB-OH or TUB-OMOM, a tomaymycin derivative, a dolastatin derivative, or a maytansinoid such as DM1 or DM4. Other anti-BCMA ADCs that can be used in the context of the methods of the present disclosure include, e.g., the ADCs referred to and known in the art as belantamab mafodotin, ispectamab debotansine, GSK2857916, AMG224, HDP-101, MEDI2228, STI-8811, and TBL-CLN1, or any of the anti-BCMA ADCs set forth, e.g., in patent publications WO2011/108008, WO2014/089335, WO2017/093942, WO2017/143069, and WO2019/025983, the contents of which are hereby incorporated by reference.

In some embodiments, the BCMA targeting agent is a chimeric antigen receptor (CAR) that binds specifically to BCMA (“BCMA CAR”). The term “chimeric antigen receptor” (CAR) refers to molecules that combine a binding domain against a component present on the target cell, for example an antibody-based specificity for a desired antigen (e.g., BCMA on plasma cell) with a T cell receptor-activating intracellular domain to generate a chimeric protein that exhibits a specific anti-target cellular immune activity. Generally, CARs consist of an extracellular single chain antibody-binding domain (scFv) fused to the intracellular signaling domain of the T cell antigen receptor complex zeta chain, and have the ability, when expressed in T cells, to redirect antigen recognition based on the monoclonal antibody's specificity. In certain embodiments, the BCMA CAR or antigen-binding fragment thereof comprises a heavy chain variable region (HCVR), light chain variable region (LCVR), and/or complementarity determining regions (CDRs) comprising the amino acid sequences of any of the antibodies set forth in WO 2020/018825, incorporated by reference. Other anti-BCMA CARs that can be used in the context of the methods of the present disclosure include, e.g., the CARs referred to and known in the art as anitocabtagene autoleucel (ACLX-001), ciltacabtagene autoleucel (JNJ-68284528), idecabtagene vicleucel (bb2121), ALLO-605, CT053, LCAR-B38M, and 4C8A, or any of the anti-BCMA CARs set forth, e.g., in patent publications WO2015/052538, WO2015/052536, WO2016/094304, WO2016/166630, WO2016/151315, WO2016/130598, WO2017/183418, WO2017/173256, WO2017211900, WO2017/130223, WO2018/229492, WO2018/085690, WO2018/151836, WO2018/028647, and WO2019/006072, incorporated by reference herein.

### Anti-BCMA x Anti-CD3 Bispecific Antibodies and Antigen-Binding Fragments Thereof

In certain exemplary embodiments of the present disclosure, the BCMA targeting agent is an antibody that specifically binds to BCMA and to CD3 (also referred to herein as an “anti-BCMA x anti-CD3 bispecific antibody” or a “BCMA x CD3 bispecific antibody”) or an antigen-binding fragment thereof. In some embodiments, the anti-BCMA x anti-CD3 bispecific antibody or antigen-binding fragment thereof is a human antibody or antigen-binding fragment thereof. In some embodiments, the anti-BCMA x anti-CD3 bispecific antibody or antigen-binding fragment thereof is a recombinant antibody (e.g., a recombinant human antibody). In some embodiments, the anti-BCMA x anti-CD3 bispecific antibody is a full antibody, e.g., an IgG1 or IgG4 antibody. In some embodiments, the anti-BCMA x anti-CD3 bispecific antibody is an IgG4 antibody.

In some embodiments, an anti-BCMA x anti-CD3 bispecific antibody or antigen-binding fragment thereof that can be used in the context of the present disclosure comprises: (a) a first antigen-binding domain that binds specifically to BCMA; and (b) a second antigen-binding domain that binds specifically to CD3. In some embodiments, the anti-BCMA x anti-CD3 bispecific antibody, or antigen-binding fragment thereof comprises a heavy chain variable region (HCVR), light chain variable region (LCVR), and/or complementarity determining regions (CDRs) comprising the amino acid sequences of any of the antibodies set forth in U.S. Pat. No. 11,384,153, incorporated by reference herein.

In some embodiments, the BCMA targeting agent is an anti-BCMA x anti-CD3 bispecific antibody or antigen-binding fragment thereof that comprises: (a) a first antigen-binding domain that binds specifically to human BCMA; and (b) a second antigen-binding domain that binds specifically to human CD3. In some embodiments, the first antigen-binding domain comprises the HCDRs of an HCVR comprising the amino acid sequence of SEQ ID NO:366 and the LCDRs of an LCVR comprising the amino acid sequence of SEQ ID NO:378. In some embodiments, the first antigen-binding domain comprises three HCDRs (HCDR1, HCDR2, and HCDR3) and three LCDRs (LCDR1, LCDR2, and LCDR3), wherein the HCDR1 comprises the amino acid sequence of SEQ ID NO:367, the HCDR2 comprises the amino acid sequence of SEQ ID NO:368, the HCDR3 comprises the amino acid sequence of SEQ ID NO:369, the LCDR1 comprises the amino acid sequence of SEQ ID NO:379, the LCDR2 comprises the amino acid sequence of SEQ ID NO:380, and the LCDR3 comprises the amino acid sequence of SEQ ID NO:381. In some embodiments, the second antigen-binding domain comprises the HCDRs of an HCVR comprising the amino acid sequence of SEQ ID NO:370 or SEQ ID NO:374 and the LCDRs of an LCVR comprising the amino acid sequence of SEQ ID NO:378. In some embodiments, the second antigen-binding domain comprises three HCDRs (HCDR1, HCDR2, and HCDR3) and three LCDRs (LCDR1, LCDR2, and LCDR3), wherein the HCDR1 comprises the amino acid sequence of SEQ ID NO:371 or SEQ ID NO:375, the HCDR2 comprises the amino acid sequence of SEQ ID NO:372 or SEQ ID NO:376, the HCDR3 comprises the amino acid sequence of SEQ ID NO:373 or SEQ ID NO:377, the LCDR1 comprises the amino acid sequence of SEQ ID NO:379, the LCDR2 comprises the amino acid sequence of SEQ ID NO:380, and the LCDR3 comprises the amino acid sequence of SEQ ID NO:381.

In some embodiments, the anti-BCMA x anti-CD3 bispecific antibody or antigen-binding fragment thereof comprises: (a) a first antigen-binding domain that comprises HCDR1, HCDR2, and HCDR3 domains, respectively, comprising the amino acid sequences of SEQ ID NOs:367, 368, and 369, and LCDR1, LCDR2, and LCDR3 domains, respectively, comprising the amino acid sequences of SEQ ID NOs:379, 380, and 381; and (b) a second antigen binding domain that comprises HCDR1, HCDR2, and HCDR3 domains, respectively, comprising the amino acid sequences of SEQ ID NOs:371, 372, and 373, and LCDR1, LCDR2, and LCDR3 domains, respectively, comprising the amino acid sequences of SEQ ID NOs:379, 380, and 381. In some embodiments, the anti-BCMA x anti-CD3 bispecific antibody or antigen-binding fragment thereof comprises: (a) a first antigen-binding domain that comprises a HCVR comprising the amino acid sequence of SEQ ID NO:366 and a LCVR comprising the amino acid sequence of SEQ ID NO:378; and (b) a second antigen-binding domain that comprises a HCVR comprising the amino acid sequence of SEQ ID NO:370 and a LCVR comprising the amino acid sequence of SEQ ID NO:378. In some embodiments, the anti-BCMA x anti-CD3 bispecific antibody or antigen-binding fragment thereof comprises: (a) a first antigen-binding domain that comprises a heavy chain comprising the amino acid sequence of SEQ ID NO:382 and a light chain comprising the amino acid sequence of SEQ ID NO:386; and (b) a second antigen-binding domain that comprises a heavy chain comprising the amino acid sequence of SEQ ID NO:383 and a light chain comprising the amino acid sequence of SEQ ID NO:386.

In some embodiments, the anti-BCMA x anti-CD3 bispecific antibody or antigen-binding fragment thereof comprises: (a) a first antigen-binding domain that comprises a HCVR having at least 90%, 91%, 92%, 93%, 94%, 95%, 96%, 97%, 98%, or 99% sequence identity to the amino acid sequence of SEQ ID NO:366 and a LCVR having at least 90%, 91%, 92%, 93%, 94%, 95%, 96%, 97%, 98%, or 99% sequence identity to the amino acid sequence of SEQ ID NO:378; and (b) a second antigen-binding domain that comprises a HCVR having at least 90%, 91%, 92%, 93%, 94%, 95%, 96%, 97%, 98%, or 99% sequence identity to the amino acid sequence of SEQ ID NO:370 and a LCVR having at least 90%, 91%, 92%, 93%, 94%, 95%, 96%, 97%, 98%, or 99% sequence identity to the amino acid sequence of SEQ ID NO:378. In some embodiments, the anti-BCMA x anti-CD3 bispecific antibody or antigen-binding fragment thereof comprises: (a) a first antigen-binding domain that comprises three HCDRs (HCDR1, HCDR2, and HCDR3) comprising the amino acid sequences of SEQ ID NOs:367, 368, and 369, respectively, and a HCVR having at least 90%, 91%, 92%, 93%, 94%, 95%, 96%, 97%, 98%, or 99% sequence identity to the amino acid sequence of SEQ ID NO:366, and comprises three LCDRs (LCDR1, LCDR2, and LCDR3) comprising the amino acid sequences of SEQ ID NOs: 379, 380, and 381, respectively, and a LCVR having at least 90%, 91%, 92%, 93%, 94%, 95%, 96%, 97%, 98%, or 99% sequence identity to the amino acid sequence of SEQ ID NO:378; and (b) a second antigen-binding domain that comprises three HCDRs (HCDR1, HCDR2, and HCDR3) comprising the amino acid sequences of SEQ ID NOs:371, 372, and 373, respectively, and a HCVR having at least 90%, 91%, 92%, 93%, 94%, 95%, 96%, 97%, 98%, or 99% sequence identity to the amino acid sequence of SEQ ID NO:370, and comprises three LCDRs (LCDR1, LCDR2, and LCDR3) comprising the amino acid sequences of SEQ ID NOs:379, 380, and 381, respectively, and a LCVR having at least 90%, 91%, 92%, 93%, 94%, 95%, 96%, 97%, 98%, or 99% sequence identity to the amino acid sequence of SEQ ID NO:378.

In some embodiments, the anti-BCMA x anti-CD3 bispecific antibody or antigen-binding fragment thereof comprises: (a) a first antigen-binding domain that comprises HCDR1, HCDR2, and HCDR3 domains, respectively, comprising the amino acid sequences of SEQ ID NOs:367, 368, and 369, and LCDR1, LCDR2, and LCDR3 domains, respectively, comprising the amino acid sequences of SEQ ID NOs:379, 380, and 381; and (b) a second antigen binding domain that comprises HCDR1, HCDR2, and HCDR3 domains, respectively, comprising the amino acid sequences of SEQ ID NOs:375, 376, and 377, and LCDR1, LCDR2, and LCDR3 domains, respectively, comprising the amino acid sequences of SEQ ID NOs:379, 380, and 381. In some embodiments, the anti-BCMA x anti-CD3 bispecific antibody or antigen-binding fragment thereof comprises: (a) a first antigen-binding domain that comprises a HCVR comprising the amino acid sequence of SEQ ID NO:366 and a LCVR comprising the amino acid sequence of SEQ ID NO:378; and (b) a second antigen-binding domain that comprises a HCVR comprising the amino acid sequence of SEQ ID NO:374 and a LCVR comprising the amino acid sequence of SEQ ID NO:378. In some embodiments, the anti-BCMA x anti-CD3 bispecific antibody or antigen-binding fragment thereof comprises: (a) a first antigen-binding domain that comprises a heavy chain comprising the amino acid sequence of SEQ ID NO:384 and a light chain comprising the amino acid sequence of SEQ ID NO:386; and (b) a second antigen-binding domain that comprises a heavy chain comprising the amino acid sequence of SEQ ID NO:385 and a light chain comprising the amino acid sequence of SEQ ID NO:386.

In some embodiments, the anti-BCMA x anti-CD3 bispecific antibody or antigen-binding fragment thereof comprises: (a) a first antigen-binding domain that comprises a HCVR having at least 90%, 91%, 92%, 93%, 94%, 95%, 96%, 97%, 98%, or 99% sequence identity to the amino acid sequence of SEQ ID NO:366 and a LCVR having at least 90%, 91%, 92%, 93%, 94%, 95%, 96%, 97%, 98%, or 99% sequence identity to the amino acid sequence of SEQ ID NO:378; and (b) a second antigen-binding domain that comprises a HCVR having at least 90%, 91%, 92%, 93%, 94%, 95%, 96%, 97%, 98%, or 99% sequence identity to the amino acid sequence of SEQ ID NO:374 and a LCVR having at least 90%, 91%, 92%, 93%, 94%, 95%, 96%, 97%, 98%, or 99% sequence identity to the amino acid sequence of SEQ ID NO:378. In some embodiments, the anti-BCMA x anti-CD3 bispecific antibody or antigen-binding fragment thereof comprises: (a) a first antigen-binding domain that comprises three HCDRs (HCDR1, HCDR2, and HCDR3) comprising the amino acid sequences of SEQ ID NOs:367, 368, and 369, respectively, and a HCVR having at least 90%, 91%, 92%, 93%, 94%, 95%, 96%, 97%, 98%, or 99% sequence identity to the amino acid sequence of SEQ ID NO:366, and comprises three LCDRs (LCDR1, LCDR2, and LCDR3) comprising the amino acid sequences of SEQ ID NOs: 379, 380, and 381, respectively, and a LCVR having at least 90%, 91%, 92%, 93%, 94%, 95%, 96%, 97%, 98%, or 99% sequence identity to the amino acid sequence of SEQ ID NO:378; and (b) a second antigen-binding domain that comprises three HCDRs (HCDR1, HCDR2, and HCDR3) comprising the amino acid sequences of SEQ ID NOs:375, 376, and 377, respectively, and a HCVR having at least 90%, 91%, 92%, 93%, 94%, 95%, 96%, 97%, 98%, or 99% sequence identity to the amino acid sequence of SEQ ID NO:374, and comprises three LCDRs (LCDR1, LCDR2, and LCDR3) comprising the amino acid sequences of SEQ ID NOs:379, 380, and 381, respectively, and a LCVR having at least 90%, 91%, 92%, 93%, 94%, 95%, 96%, 97%, 98%, or 99% sequence identity to the amino acid sequence of SEQ ID NO:378.

An exemplary antibody, comprising a BCMA-binding heavy chain comprising the amino acid sequence of SEQ ID NO:382, a CD3-binding heavy chain comprising the amino acid sequence of SEQ ID NO:383, and a light chain comprising the amino acid sequence of SEQ ID NO:386, is the BCMA x CD3 bispecific antibody known as linvoseltamab (also known as REGN5458). Another exemplary antibody, comprising a BCMA-binding heavy chain comprising the amino acid sequence of SEQ ID NO:384, a CD3-binding heavy chain comprising the amino acid sequence of SEQ ID NO:385, and a light chain comprising the amino acid sequence of SEQ ID NO:386, is the BCMA x CD3 bispecific antibody known as vonsetamig (also known as REGN5459).

According to certain exemplary embodiments, the methods of the present disclosure comprise the use of linvoseltamab or vonsetamig. As used herein, “linvoseltamab” also includes bioequivalents of linvoseltamab, and “vonsetamig” also includes bioequivalents of vonsetamig. The term “bioequivalent,” as used herein with reference to linvoseltamab or vonsetamig, refers to BCMA x CD3 bispecific antibodies or antigen-binding fragments thereof that are pharmaceutical equivalents or pharmaceutical alternatives whose rate and/or extent of absorption do not show a significant difference with that of linvoseltamab or vonsetamig when administered at the same molar dose under similar experimental conditions, either single dose or multiple doses. In some embodiments, the term refers to antigen-binding proteins that bind to BCMA and CD3 which do not have clinically meaningful differences with linvoseltamab or vonsetamig in their safety, purity and/or potency.

Other anti-BCMA x anti-CD3 antibodies that can be used in the context of the methods of the present disclosure include, but are not limited to, alnuctamab (BMS-986349/CC-93269), elranatamab (PF-06863135), teclistamab (JNJ-64007957), pacanalotamab (AMG420), pavurutamab (AMG701), CM336, EM801, EMB-06, HBM7020, TNB-383B (ABBV-383), TNB-384B, TQB2934, YKST02, or any of the anti-BCMA x anti-CD3 antibodies set forth, e.g., in U.S. Pat. Nos. 9,969,809, 10,072,088, 11,505,606, or in patent publications WO2013/072415, WO2014/140248, WO2014/122144, WO2016/166629, WO2016/079177, WO2016/020332, WO2017031104, WO2017/223111, WO2017/134134, WO2018/083204, WO2018/201051, incorporated by reference herein.

### Sequence Variants

The antibodies and antigen-binding fragments of the present disclosure may comprise one or more amino acid substitutions, insertions, and/or deletions (e.g., 1, 2, 3, 4, 5, 6, 7, 8, 9, or 10 substitutions and/or 1, 2, 3, 4, 5, 6, 7, 8, 9, or 10 insertions and/or 1, 2, 3, 4, 5, 6, 7, 8, 9, or 10 deletions) in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. Methods involving the use of antibodies, and antigen-binding fragments thereof, that are derived from any of the amino acid sequences disclosed herein, wherein one or more amino acids (e.g., 1, 2, 3, 4, 5, 6, 7, 8, 9, or 10 amino acids) within one or more framework and/or one or more (e.g., 1, 2, 3, 4, 5, 6, 7, 8, 9, 10, 11 or 12 with respect to the tetrameric antibody or 1, 2, 3, 4, 5 or 6 with respect to the HCVR and LCVR of an antibody) CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”), are provided. A person of ordinary skill in the art, starting with the heavy and light chain variable region sequences disclosed herein, can easily produce numerous antibodies and antigen-binding fragments that comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2, or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired properties, such as improved binding specificity, increased binding affinity, improved or enhanced antagonistic or agonistic biological properties (as the case may be), reduced immunogenicity, etc.

In some embodiments, the present disclosure includes antibodies and antigen-binding fragments thereof that comprise variants of any of the HCVR, LCVR, and/or CDR amino acid sequences disclosed herein having one or more conservative substitutions. For example, the present disclosure includes antigen-binding molecules comprising an antigen-binding domain having HCVR, LCVR, and/or CDR amino acid sequences with, e.g., 10 or fewer, 9 or fewer, 8 or fewer, 7 or fewer, 6 or fewer, 5 or fewer, 4 or fewer, 3 or fewer, 2 or fewer, or 1 conservative amino acid substitution(s) relative to any of the HCVR, LCVR, and/or CDR amino acid sequences disclosed herein. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256:1443-1445. A “moderately conservative” replacement is any change having a nonnegative value in the PAM250 log-likelihood matrix.

In some embodiments, the present disclosure includes antibodies and antigen-binding fragments thereof comprising an HCVR, LCVR, and/or CDR amino acid sequence that is substantially identical to any of the HCVR, LCVR, and/or CDR amino acid sequences disclosed herein. In some embodiments, an antibody or antigen-binding fragment comprises HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 1. In some embodiments, an antibody or antigen-binding fragment comprises HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 1, wherein the differences in the amino acid residue(s) relative to the sequence disclosed in Table 1 are conservative substitutions or moderately conservative substitutions.

In some embodiments, an antibody (such as an anti-IL-4R antibody as disclosed herein) does not have a C-terminal lysine in the heavy chain. For example, a C-terminal lysine that is present at the end of a heavy chain sequence may be removed as a post-translational modification during manufacture, e.g., during protein expression. Alternatively, a C-terminal lysine may be removed by recombinant technology (e.g., the coding sequence of the heavy chain does not include a codon for a C-terminal lysine). Thus, contemplated within the present disclosure are antibodies comprising a heavy chain in which a C-terminal lysine, if included in the amino acid sequence (e.g., as in SEQ ID NO:9) is absent (e.g., as in SEQ ID NO:387).

### Preparation of Human Antibodies

Methods for generating human antibodies in transgenic mice are known in the art. Any such known methods can be used in the context of the present disclosure to make human antibodies that specifically bind to an antigen of interest, e.g., human IL-4R, human BCMA, or human CD3.

Using VELOCIMMUNE™ technology (see, for example, U.S. Pat. No. 6,596,541, Regeneron Pharmaceuticals) or any other known method for generating monoclonal antibodies, high affinity chimeric antibodies to an antigen are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In general, the antibodies that can be used in the methods of the present disclosure possess high affinities, as described above, when measured by binding to antigen either immobilized on solid phase or in solution phase. The mouse constant regions are replaced with desired human constant regions to generate the fully human antibodies of the disclosure. While the constant region selected may vary according to specific use, high affinity antigen-binding and target specificity characteristics reside in the variable region.

### Pharmaceutical Compositions and Kits

In one aspect, the present disclosure provides methods that comprise administering an IL-4R antagonist and a BCMA targeting agent to a subject, wherein the IL-4R antagonist (e.g., an anti-IL-4R antibody) and/or the BCMA targeting agent (e.g., an anti-BCMA x anti-CD3 bispecific antibody) is contained within one or more pharmaceutical compositions that comprises one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, PA. In some embodiments, the carrier is suitable for intravenous, intramuscular, oral, intraperitoneal, intrathecal, transdermal, topical, or subcutaneous administration.

Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, a pharmaceutical composition of the present disclosure is contained within a container. Thus, in another aspect, containers comprising an IL-4R antagonist and/or the BCMA targeting agent, or a pharmaceutical composition comprising an IL-4R antagonist and/or the BCMA targeting agent, are provided. For example, in some embodiments, a pharmaceutical composition is contained within a container selected from the group consisting of a glass vial, a syringe, a pen delivery device, and an autoinjector.

In some embodiments, a pharmaceutical composition of the present disclosure is delivered, e.g., subcutaneously or intravenously, with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. Typically, a reusable pen delivery device utilizes a replaceable cartridge that contains a pharmaceutical composition. Once the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany). Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park Ill.).

In some embodiments, the pharmaceutical composition is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science, 249:1527-1533. Other delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu, et al., 1987, J. Biol. Chem., 262:4429-4432).

In some embodiments, a pharmaceutical composition comprising an anti-IL-4R antibody is administered using a drug delivery device that is a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An exemplary sleeve-triggered auto-injector with manual needle insertion is described in International Publication WO2015/004052. Exemplary audible end-of-dose feedback mechanisms are described in International Publications WO2016/193346 and WO2016/193348. An exemplary needle-safety mechanism after using an auto-injector is described in International Publication WO2016/193352. An exemplary needle sheath remover mechanism for a syringe auto-injector is described in International Publication WO2016/193353. An exemplary support mechanism for supporting an axial position of a syringe is described in International Publication WO2016/193355.

In some embodiments, pharmaceutical compositions for use as described herein are prepared into dosage forms in a unit dose suited to fit a dose of the active ingredients. Such dosage forms in a unit dose include, for example, a vial or a prefilled syringe.

In another aspect, the present disclosure provides kits for administering an IL-4R antagonist and a BCMA targeting agent to a subject, e.g., for administering to a subject having an IgE-mediated food allergy, or for use in treating a subject having an IgE-mediated food allergy or preventing or reducing the severity of an allergic reaction in a subject having an IgE-mediated food allergy. In some embodiments, the kit comprises an IL-4R antagonist (e.g., an anti-IL-4R antibody as disclosed herein) and a BCMA targeting agent (e.g., an anti-BCMA x anti-CD3 bispecific antibody as disclosed herein). In some embodiments, the kit further comprises instructions for use, e.g., instructions for administering the IL-4R antagonist and the BCMA targeting agent to a subject in need thereof.

### Dosage and Administration

In some embodiments, an IL-4R antagonist (e.g., an anti-IL-4R antibody) and a BCMA targeting agent (e.g., a BCMA x CD3 bispecific antibody) are administered to a subject in need thereof (e.g., a subject having a food allergy) according to the methods of the present disclosure in a therapeutically effective amount. As used herein, the phrase “therapeutically effective amount” means an amount of an active agent (e.g., IL-4R antagonist, BCMA targeting agent, or a combination thereof) that results in one or more of: (a) a reduction in the occurrence, severity, or duration of a symptom of an allergy; (b) reduction in the level of serum allergen-specific IgE (e.g., a food allergen-specific IgE or a perennial allergen-specific IgE); (c) elimination or reduction to an undetectable level of a serum immunoglobulin (e.g., IgE, IgG, IgA, or IgM); (d) reduction of allergen sensitization (e.g., as measured by a skin prick test or by level of serum allergen-specific IgE); (e) reduction in susceptibility to an allergic reaction; and/or (f) a reduction in the use or need for conventional anti-allergy therapy (e.g., reduced or eliminated use of corticosteroids) or rescue medication (e.g., epinephrine) as compared to an untreated subject or a subject treated with either therapeutic agent as monotherapy.

In some embodiments, prior to the administration of the BCMA targeting agent, the IL-4R antagonist (e.g., an anti-IL-4R antibody such as dupilumab) is administered to the subject for a length of time sufficient to attain a steady state concentration of the IL-4R antagonist. In some embodiments, prior to the administration of the BCMA targeting agent, the method comprises determining the concentration of the IL-4R antagonist in a sample from the subject. In some embodiments, the concentration of the IL-4R antagonist (e.g., an anti-IL-4R antibody such as dupilumab) is measured in a serum sample from the subject, e.g., using an immunoassay such as an ELISA assay. See, e.g., Davis et al., Clin. Pharmacol. Ther. 2018, 104:1146-1154.

As used herein, the term “steady state” refers to a pharmacokinetic condition in which the rate of drug input equals the rate of drug elimination. As will be recognized by a person of ordinary skill in the art, the length of time required for a drug (e.g., antibody) to reach steady state depends upon several factors (see, e.g., Wadhwa and Cascella, “Steady State Concentration” in StatPearls, 2023, incorporated by reference herein). In some embodiments, the length of time sufficient to attain a steady state concentration of the IL-4R antagonist (e.g., an anti-IL-4R antibody such as dupilumab) is at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, or longer. In some embodiments, the length of time sufficient to attain a steady state concentration of the IL-4R antagonist is about 12 weeks. In some embodiments, the length of time sufficient to attain a steady state concentration of the IL-4R antagonist is about 16 weeks. (See, e.g., Jackson et al., Annals of Allergy, Asthma & Immunology, 2023, 131:44-51; Dellon et al., Clin Trans Gastroenterol 2024, 16(1):e00793.)

In some embodiments, the IL-4R antagonist is administered to the subject for at least 4 weeks, at least 6 weeks, at least 8 weeks, at least 10 weeks, at least 12 weeks, at least 14 weeks, at least 16 weeks, at least 18 weeks, at least 20 weeks or longer prior to the first administration of the BCMA targeting agent. In some embodiments, the IL-4R antagonist (e.g., an anti-IL-4R antibody) is administered as a long-term background therapy (e.g., for at least 6 months, at least 1 year, at least 1.5 years, at least 2 years, or longer).

In the case of an IL-4R antagonist (e.g., an anti-IL-4R antibody such as dupilumab), a therapeutically effective amount can be from about 10 mg to about 800 mg, e.g., about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, or about 800 mg of the anti-IL-4R antibody. In some embodiments, a therapeutically effective amount is from about 50 mg to about 600 mg, or from about 100 mg to about 800 mg, or from about 100 mg to about 600 mg, or from about 200 mg to about 600 mg. In certain embodiments, 50 mg, 75 mg, 100 mg, 125 mg, 150 mg, 200 mg, 250 mg, 300 mg, 350 mg, 400 mg, 450 mg, 500 mg, 550 mg, 600 mg, 650 mg, 700 mg, 750 mg, or 800 mg of an IL-4R antagonist (e.g., an anti-IL-4R antibody such as dupilumab) is administered to a subject. In some embodiments, the IL-4R antagonist (e.g., an anti-IL-4R antibody such as dupilumab) administered once a week (QW), once every two weeks (Q2W), once every three weeks (Q3W), once every four weeks (Q4W), once every five weeks (Q5W), once every six weeks (Q6W), once every seven weeks (Q7W), or once every eight weeks (Q8W). In some embodiments, the IL-4R antagonist (e.g., an anti-IL-4R antibody such as dupilumab) administered once a month, once every two months, once every three months, or once every four months.

The amount of IL-4R antagonist (e.g., anti-IL-4R antibody) contained within the individual doses may be expressed in terms of milligrams of antibody per kilogram of subject body weight (i.e., mg/kg). For example, the IL-4R antagonist may be administered to a subject at a dose of about 0.0001 to about 10 mg/kg of subject body weight, e.g., at a dose of about 1 mg/kg to about 10 mg/kg, at a dose of about 2 mg/kg to about 9 mg/kg, or at a dose of about 3 mg/kg to about 8 mg/kg. In some embodiments, the IL-4R antagonist may be administered to a subject at a dose of about 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg.

In some embodiments, the BCMA targeting agent (e.g., a BCMA x CD3 bispecific antibody such as linvoseltamab) is administered in an amount sufficient to reduce the concentration of total IgE (e.g., serum total IgE) by at least 40%, at least 50%, at least 60%, at least 70%, at least 80%, or more, e.g., relative to the subject's baseline level of serum total IgE. In some embodiments, the BCMA targeting agent (e.g., a BCMA x CD3 bispecific antibody such as linvoseltamab) is administered in an amount sufficient to reduce the concentration of total IgE (e.g., serum total IgE) to a level that is below the lower limit of quantification (LLOQ) (e.g., to a serum total IgE level <2 kU/L). In some embodiments, the BCMA targeting agent is administered (e.g., as a single dose or in multiple doses) in an amount sufficient to reduce the concentration of total IgE to a level that is below the LLOQ, then administration of the BMCA targeting agent is stopped.

In the case of a BCMA x CD3 bispecific antibody (e.g., linvoseltamab), a therapeutically effective amount can be from about 0.05 mg to about 500 mg, from about 0.10 mg to about 300 mg, from about 0.10 mg to about 200 mg, from about 0.10 mg to about 100 mg, from about 0.10 mg to about 50 mg, from about 0.15 mg to about 250 mg, from about 0.25 mg to about 400 mg, from about 0.3 mg to about 350 mg, from about 0.5 mg to about 300 mg of the antibody, from about 0.5 mg to about 200 mg, from about 0.5 mg to about 100 mg, from about 0.5 mg to about 50 mg, from about 1 mg to about 500 mg, from about 1 mg to about 250 mg, from about 1 mg to about 100 mg, from about 1 mg to about 50 mg, from about 5 mg to about 80 mg, or from about 5 mg to about 40 mg. For example, in various embodiments, the amount of the BCMA x CD3 bispecific antibody is about 0.05 mg, about 0.1 mg, about 0.15 mg, about 0.2 mg, about 0.25 mg, about 0.3 mg, about 0.4 mg, about 0.5 mg, about 0.6 mg, about 0.7 mg, about 0.75 mg, about 0.8 mg, about 0.9 mg, about 1 mg, about 1.5 mg, about 2 mg, about 3 mg, about 4 mg, about 5 mg, about 6 mg, about 7 mg, about 8 mg, about 9 mg, about 10 mg, about 15 mg, about 20 mg, about 25 mg, about 30 mg, about 40 mg, about 50 mg, about 60 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg of the antibody.

In some embodiments, a single dose of the BCMA targeting agent (e.g., a BCMA x CD3 bispecific antibody) is administered. In some embodiments, multiple doses of the BCMA targeting agent (e.g., a BCMA x CD3 bispecific antibody) are administered. In some embodiments, the BCMA targeting agent (e.g., a BCMA x CD3 bispecific antibody) is administered to a subject at a dosing frequency of about four times a week, twice a week, once a week, once every two weeks, once every three weeks, once every four weeks, once every five weeks, once every six weeks, once every eight weeks, once every ten weeks, once every twelve weeks, or less frequently so long as a therapeutic response is achieved. In some embodiments, the methods disclosed herein comprise administering the BCMA targeting agent to a subject once every week, once every two weeks, once every three weeks, or once every four weeks. In some embodiments, the BCMA targeting agent (e.g., a BCMA x CD3 bispecific antibody) is administered as a long-term background therapy.

In some embodiments, the methods of the disclosure comprise sequentially administering to the subject a single initial dose of an IL-4R antagonist and/or BCMA targeting agent, followed by one or more secondary doses of the IL-4R antagonist and/or BCMA targeting agent, and optionally followed by one or more tertiary doses of the IL-4R antagonist and/or BCMA targeting agent.

In some embodiments, the methods of the disclosure comprise sequentially administering to the subject a single initial dose of an IL-4R antagonist (e.g., an IL-4R antibody such as but not limited to dupilumab), optionally followed by one or more secondary doses of the IL-4R antagonist, before the first administration of a BCMA targeting agent (e.g., a BCMA x CD3 bispecific antibody such as but not limited to linvoseltamab or vonsetamig). In some embodiments, at least 2, 3, 4, 5, 6, 7, or 8 doses of the IL-4R antagonist are administered to the subject before the first administration of the BCMA targeting agent. In some embodiments, the subject is on a background therapy of the IL-4R antagonist prior to the first administration of the BCMA targeting agent.

In some embodiments, the methods of the disclosure comprise sequentially administering to the subject a single initial dose of a BCMA targeting agent (e.g., a BCMA x CD3 bispecific antibody such as but not limited to linvoseltamab or vonsetamig), optionally followed by one or more secondary doses of the BCMA targeting agent, before the first administration of an IL-4R antagonist (e.g., an IL-4R antibody such as but not limited to dupilumab). In some embodiments, at least 2, 3, 4, 5, 6, 7, or 8 doses of the BCMA targeting agent are administered to the subject before the first administration of the IL-4R antagonist. In some embodiments, the subject is on a background therapy of the BCMA targeting agent prior to the first administration of the IL-4R antagonist.

The terms “initial dose,” “secondary doses,” and “tertiary doses,” refer to the temporal sequence of administration of the therapeutic agent. Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. The initial, secondary, and tertiary doses may all contain the same amount of therapeutic agent, but generally may differ from one another in terms of frequency of administration. In certain embodiments, however, the amount of therapeutic agent contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). In some embodiments, the initial or loading dose and the one or more secondary or maintenance doses each contain the same amount of the therapeutic agent. In other embodiments, the initial dose comprises a first amount of the therapeutic agent, and the one or more secondary doses each comprise a second amount of the therapeutic agent. For example, the first amount of the therapeutic agent can be 1.5×, 2×, 2.5×, 3×, 3.5×, 4× or 5× or more than the second amount of the therapeutic agent. In some embodiments, one or more maintenance doses of the therapeutic agent are administered without a loading dose.

In some embodiments, a loading dose is a “split dose” that is administered as two or more doses (e.g., 2, 3, 4, or 5 doses) that are administered on separate days. In some embodiments, a loading dose is administered as a split dose wherein the two or more doses are administered at least about one week apart. In some embodiments, a loading dose is administered as a split dose wherein the two or more doses are administered about 1 week, 2 weeks, 3 weeks, or 4 weeks apart. In some embodiments, the loading dose is split evenly over the two or more doses (e.g., half of the loading dose is administered as the first portion and half of the loading dose is administered as the second portion). In some embodiments, the loading dose is split unevenly over the two or more doses (e.g., more than half of the loading dose is administered as the first portion and less than half of the loading dose is administered as the second portion).

In some embodiments, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of the therapeutic agent which is administered to a patient prior to the administration of the very next dose in the sequence with no intervening doses.

In some embodiments involving multiple secondary doses, each secondary dose is administered at the same frequency as the other secondary doses. For example, each secondary dose may be administered to the subject 1 week, 2 weeks, 3 weeks, or 4 weeks after the immediately preceding dose. Similarly, in some embodiments involving multiple tertiary doses, each tertiary dose is administered at the same frequency as the other tertiary doses. For example, each tertiary dose may be administered to the subject 1 week, 2 weeks, 3 weeks, or 4 weeks after the immediately preceding dose. Alternatively, the frequency at which the secondary and/or tertiary doses are administered to a subject can vary over the course of the treatment regimen. The frequency of administration may also be adjusted during the course of treatment by a physician depending on the needs of the individual patient following clinical examination.

In some embodiments, an IL-4R antagonist (e.g., an anti-IL-4R antibody such as dupilumab) is administered to the subject for at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, or longer before the first administration of the BCMA targeting agent (e.g., a BCMA x CD3 bispecific antibody).

In some embodiments, the IL-4R antagonist (e.g., anti-IL-4R antibody such as dupilumab) is administered to the subject for at least 6 weeks (e.g., at least 8 weeks, at least 10 weeks, at least 12 weeks, at least 14 weeks, at least 16 weeks, at least 18 weeks, at least 20 weeks, or longer) at a dose of 300 mg with or without a loading dose (e.g., a loading dose of 600 mg) before the first administration of the BCMA targeting agent (e.g., BCMA x CD3 bispecific antibody). In some embodiments, the IL-4R antagonist is administered at a dose of 300 mg QW, with or without a loading dose. In some embodiments, the IL-4R antagonist is administered at a dose of 300 mg Q2W, with or without a loading dose. In some embodiments, the IL-4R antagonist is administered at a dose of 300 mg Q3W, with or without a loading dose. In some embodiments, the IL-4R antagonist is administered at a dose of 300 mg Q4W, with or without a loading dose.

In some embodiments, the IL-4R antagonist (e.g., anti-IL-4R antibody such as dupilumab) is administered to the subject for at least 6 weeks (e.g., at least 8 weeks, at least 10 weeks, at least 12 weeks, at least 14 weeks, at least 16 weeks, at least 18 weeks, at least 20 weeks, or longer) at a dose of 200 mg with or without a loading dose (e.g., a loading dose of 400 mg) before the first administration of the BCMA targeting agent (e.g., BCMA x CD3 bispecific antibody). In some embodiments, the IL-4R antagonist is administered at a dose of 200 mg QW, with or without a loading dose. In some embodiments, the IL-4R antagonist is administered at a dose of 200 mg Q2W, with or without a loading dose. In some embodiments, the IL-4R antagonist is administered at a dose of 200 mg Q3W, with or without a loading dose. In some embodiments, the IL-4R antagonist is administered at a dose of 200 mg Q4W, with or without a loading dose.

### Additional Therapeutic Agents

In some embodiments, the present disclosure further provides for the administration of one or more therapeutic agents in addition to the IL-4R antagonist and the BCMA targeting agent. In some embodiments, the additional therapeutic agent(s) is a rescue medication (e.g., for the treatment of one or more signs or symptoms of an allergic reaction). In some embodiments, the additional therapeutic agent(s) is administered before, concurrent with, or after the IL-4R antagonist and/or the BCMA targeting agent.

For example, in some embodiments, the additional therapeutic agent is administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the IL-4R antagonist and/or the BCMA targeting agent. In some embodiments, the additional therapeutic agent is administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the IL-4R antagonist and/or the BCMA targeting agent. In some embodiments, the additional therapeutic agent is administered within 10 minutes or within 5 minutes of the administration of the IL-4R antagonist and/or the BCMA targeting agent.

In some embodiments, the additional therapeutic agent is a steroid, an antihistamine, a decongestant, or an analgesic. In some embodiments, the additional therapeutic agent is a steroid (e.g., a corticosteroid, such as an inhaled corticosteroid (ICS), oral corticosteroid (OCS), intravenous corticosteroid, intramuscular corticosteroid, or subcutaneous corticosteroid, e.g., dexamethasone). In some embodiments, the additional therapeutic agent is an antihistamine (e.g., loratadine, fexofenadine, cetirizine, diphenhydramine, promethazine, carbinoxamine, desloratadine, hydroxyzine, levocetirizine, triprolidine, brompheniramine, or chlorpheniramine). In some embodiments, the additional therapeutic agent is a decongestant (e.g., pseudoephedrine or phenylephrine). In some embodiments, the additional therapeutic agent is an analgesic (e.g., acetaminophen, paracetamol, or a nonsteroidal anti-inflammatory drug). In some embodiments, the additional therapeutic agent is epinephrine.

TABLE 1
Informal Sequence Listing

| SEQ | | |
|---|---|---|
| ID NO | Sequence | Description |
| 1 | EVQLVESGGGLEQPGGSLRLSCAGSGFTFRDYAMTWVRQAPGKGLEWVSSISGSG | Dupilumab HCVR amino |
| | GNTYYADSVKGRFTISRDNSKNTLYLQMNSLRAEDTAVYYCAKDRLSITIRPRYYGLD | acid sequence |
| | VWGQGTTVTVS | |
| 2 | DIVMTQSPLSLPVTPGEPASISCRSSQSLLYSIGYNYLDWYLQKSGQSPQLLIYLGSNR | Dupilumab LCVR amino |
| | ASGVPDRFSGSGSGTDFTLKISRVEAEDVGFYYCMQALQTPYTFGQGTKLEIK | acid sequence |
| 3 | GFTFRDYA | Dupilumab HCDR1 |
| | | amino acid sequence |
| 4 | ISGSGGNT | Dupilumab HCDR2 |
| | | amino acid sequence |
| 5 | AKDRLSITIRPRYYGLDV | Dupilumab HCDR3 |
| | | amino acid sequence |
| 6 | QSLLYSIGYNY | Dupilumab LCDR1 amino |
| | | acid sequence |
| 7 | LGS | Dupilumab LCDR2 amino |
| | | acid sequence |
| 8 | MQALQTPYT | Dupilumab LCDR3 amino |
| | | acid sequence |
| 9 | EVQLVESGGGLEQPGGSLRLSCAGSGFTFRDYAMTWVRQAPGKGLEWVSSISGSG | Dupilumab heavy chain |
| | GNTYYADSVKGRFTISRDNSKNTLYLQMNSLRAEDTAVYYCAKDRLSITIRPRYYGLD | amino acid sequence |
| | VWGQGTTVTVSSASTKGPSVFPLAPCSRSTSESTAALGCLVKDYFPEPVTVSWNSGA | |
| | LTSGVHTFPAVLQSSGLYSLSSVVTVPSSSLGTKTYTCNVDHKPSNTKVDKRVESKYGP | |
| | PCPPCPAPEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSLGK | |
| 10 | DIVMTQSPLSLPVTPGEPASISCRSSQSLLYSIGYNYLDWYLQKSGQSPQLLIYLGSNR | Dupilumab light chain |
| | ASGVPDRFSGSGSGTDFTLKISRVEAEDVGFYYCMQALQTPYTFGQGTKLEIKRTVAA | amino acid sequence |
| | PSVFIFPPSDEQLKSGTASVVCLLNNFYPREAKVQWKVDNALQSGNSQESVTEQDSK | |
| | DSTYSLSSTLTLSKADYEKHKVYACEVTHQGLSSPVTKSFNRGEC | |
| 11 | MKVLQEPTCVSDYMSISTCEWKMNGPTNCSTELRLLYQLVFLLSEAHTCIPENNGGA | Human IL-4Rα |
| | GCVCHLLMDDVVSADNYTLDLWAGQQLLWKGSFKPSEHVKPRAPGNLTVHTNVS | |
| | DTLLLTWSNPYPPDNYLYNHLTYAVNIWSENDPADFRIYNVTYLEPSLRIAASTLKSGI | |
| | SYRARVRAWAQCYNTTWSEWSPSTKWHNSYREPFEQH | |
| 12 | EIVLTQSPGTLSLSPGERATLSCRASQSVSNSYLAWYQQKPGQAPRLLIFGASSRATGI | SCB-VL-39 |
| | PDRFSGSGSGTDFTLTISRLEPEDFAVYYCQQYGSSPPWTFGQGTKVEIK | |
| 13 | EIVLTQSPGTLSLSPGERATLSCRASQSVSSSYLAWYQQKPGQAPRLLIYGASSRATGI | SCB-VL-40 |
| | PDRFSGSGSGTDFTLTISRLEPEDFAVYYCQQYGSSPPWTFGQGTKVEIK | |
| 14 | EIVLTQSPGTLSLSPGERATLSCRASQSVSSSYLAWYQQKPGQAPRLLIFGASSRAPGI | SCB-VL-41 |
| | PDRFSGSGSGTDFTLTISRLEPEDFAVYYCQQYGSSPPWTFGQGTKVEIK | |
| 15 | EIVLTQSPGTLSLSPGERATLSCRASQSVSNSYLAWYQQKPGQAPRLLIYGASSRATGI | SCB-VL-42 |
| | PDRFSGSGSGTDFTLTISRLEPEDFAVYYCQQYGSSPPWTFGQGTKVEIK | |
| 16 | EIVLTQSPGTLSLSPGERATLSCRASQSVSNSYLAWYQQKPGQAPRLLIFGASSRAPGI | SCB-VL-43 |
| | PDRFSGSGSGTDFTLTISRLEPEDFAVYYCQQYGSSPPWTFGQGTKVEIK | |
| 17 | EIVLTQSPGTLSLSPGERATLSCRASQSVSSSYLAWYQQKPGQAPRLLIYGASSRAPGI | SCB-VL-44 |
| | PDRFSGSGSGTDFTLTISRLEPEDFAVYYCQQYGSSPPWTFGQGTKVEIK | |
| 18 | EIVLTQSPGTLSLSPGERATLSCRASQSVSSSYLAWYQQKPGQAPRLLIFGASSRATGI | SCB-VL-45 |
| | PDRFSGSGSGTDFTLTISRLEPEDFAVYYCQQYDHSPPWTFGQGTKVEIK | |
| 19 | EIVLTQSPGTLSLSPGERATLSCRASQSVSSSYLAWYQQKPGQAPRLLIFGASSRATGI | SCB-VL-46 |
| | PDRFSGSGSGTDFTLTISRLEPEDFAVYYCQQYGSSAGWTFGQGTKVEIK | |
| 20 | EIVLTQSPGTLSLSPGERATLSCRASQSVSSSYLAWYQQKPGQAPRLLIFGASSRATGI | SCB-VL-47 |
| | PDRFSGSGSGTDFTLTISRLEPEDFAVYYCQQYDHSAGWTFGQGTKVEIK | |
| 21 | EIVLTQSPGTLSLSPGERATLSCRASQSVSNSYLAWYQQKPGQAPRLLIFGASSRATGI | SCB-VL-48 |
| | PDRFSGSGSGTDFTLTISRLEPEDFAVYYCQQYDHSPPWTFGQGTKVEIK | |
| 22 | EIVLTQSPGTLSLSPGERATLSCRASQSVSSSYLAWYQQKPGQAPRLLIYGASSRATGI | SCB-VL-49 |
| | PDRFSGSGSGTDFTLTISRLEPEDFAVYYCQQYDHSPPWTFGQGTKVEIK | |
| 23 | EIVLTQSPGTLSLSPGERATLSCRASQSVSSSYLAWYQQKPGQAPRLLIFGASSRAPGI | SCB-VL-50 |
| | PDRFSGSGSGTDFTLTISRLEPEDFAVYYCQQYDHSPPWTFGQGTKVEIK | |
| 24 | EIVLTQSPGTLSLSPGERATLSCRASQSVSSSYLAWYQQKPGQAPRLLIYGASSRAPGI | SCB-VL-51 |
| | PDRFSGSGSGTDFTLTISRLEPEDFAVYYCQQYDHSAGWTFGQGTKVEIK | |
| 25 | EIVLTQSPGTLSLSPGERATLSCRASQSVSNSYLAWYQQKPGQAPRLLIFGASSRAPGI | SCB-VL-52 |
| | PDRFSGSGSGTDFTLTISRLEPEDFAVYYCQQYDHSAGWTFGQGTKVEIK | |
| 26 | EIVLTQSPGTLSLSPGERATLSCRASQSVSNSYLAWYQQKPGQAPRLLIYGASSRATGI | SCB-VL-53 |
| | PDRFSGSGSGTDFTLTISRLEPEDFAVYYCQQYDHSAGWTFGQGTKVEIK | |
| 27 | EIVLTQSPGTLSLSPGERATLSCRASQSVSSSYLAWYQQKPGQAPRLLIFGASSRAPGI | SCB-VL-54 |
| | PDRFSGSGSGTDFTLTISRLEPEDFAVYYCQQYDHSAGWTFGQGTKVEIK | |
| 28 | EIVLTQSPGTLSLSPGERATLSCRASQSVSSSYLAWYQQKPGQAPRLLIYGASSRATGI | SCB-VL-55 |
| | PDRFSGSGSGTDFTLTISRLEPEDFAVYYCQQYDHSAGWTFGQGTKVEIK | |
| 29 | EIVLTQSPGTLSLSPGERATLSCRASQSVSNSYLAWYQQKPGQAPRLLIFGASSRATGI | SCB-VL-56 |
| | PDRFSGSGSGTDFTLTISRLEPEDFAVYYCQQYDHSAGWTFGQGTKVEIK | |
| 30 | EIVLTQSPGTLSLSPGERATLSCRASQSVSSSYLAWYQQKPGQAPRLLIFGASSRATGI | SCB-VL-57 |
| | PDRFSGSGSGTDFTLTISRLEPEDFAVYYCQQYGSSPPWTFGQGTKVEIK | |
| 31 | EIVLTQSPGTLSLSPGERATLSCRASQSVSNSYLAWYQQKPGQAPRLLIYGASSRAPGI | SCB-VL-58 |
| | PDRFSGSGSGTDFTLTISRLEPEDFAVYYCQQYDHSAGWTFGQGTKVEIK | |
| 32 | EVQLVESGGGLVHPGGSLRLSCAGSGFTFSRNAMFWVRQAPGKGLEWVSGIGTGG | SCB-VH-59 |
| | ATNYADSVKGRFTISRDNAKNSLYLQMNSLRAEDMAVYYCARGRYYFDYWGQGTL | |
| | VTVSS | |
| 33 | EVQLVQSGGGLVQPGGSLRLSCAGSGFTFSRNAMFWVRQAPGKGLEWVSGIGTG | SCB-VH-60 |
| | GATNYADSVKGRFTISRDNAKNSLYLQMNSLRAEDMAVYYCARGRYYFDYWGQGT | |
| | LVTVSS | |
| 34 | EVQLVQSGGGLVHPGGSLRLSCAASGFTFSRNAMFWVRQAPGKGLEWVSGIGTGG | SCB-VH-61 |
| | ATNYADSVKGRFTISRDNAKNSLYLQMNSLRAEDMAVYYCARGRYYFDYWGQGTL | |
| | VTVSS | |
| 35 | EVQLVQSGGGLVHPGGSLRLSCAGSGFTFSRNAMFWVRQAPGKGLEWVSGIGTGG | SCB-VH-62 |
| | ATSYADSVKGRFTISRDNAKNSLYLQMNSLRAEDMAVYYCARGRYYFDYWGQGTLV | |
| | TVSS | |
| 36 | EVQLVQSGGGLVHPGGSLRLSCAGSGFTFSRNAMFWVRQAPGKGLEWVSGIGTGG | SCB-VH-63 |
| | ATNYADSVKGRFTISRDNAKNSLYLQMNSLRAEDTAVYYCARGRYYFDYWGQGTLV | |
| | TVSS | |
| 37 | EVQLVESGGGLVQPGGSLRLSCAGSGFTFSRNAMFWVRQAPGKGLEWVSGIGTGG | SCB-VH-64 |
| | ATNYADSVKGRFTISRDNAKNSLYLQMNSLRAEDMAVYYCARGRYYFDYWGQGTL | |
| | VTVSS | |
| 38 | EVQLVESGGGLVHPGGSLRLSCAASGFTFSRNAMFWVRQAPGKGLEWVSGIGTGG | SCB-VH-65 |
| | ATNYADSVKGRFTISRDNAKNSLYLQMNSLRAEDMAVYYCARGRYYFDYWGQGTL | |
| | VTVSS | |
| 39 | EVQLVQSGGGLVQPGGSLRLSCAASGFTFSRNAMFWVRQAPGKGLEWVSGIGTGG | SCB-VH-66 |
| | ATNYADSVKGRFTISRDNAKNSLYLQMNSLRAEDMAVYYCARGRYYFDYWGQGTL | |
| | VTVSS | |
| 40 | EVQLVQSGGGLVHPGGSLRLSCAGSGFTFSRNAMFWVRQAPGKGLEWVSGIGTGG | SCB-VH-67 |
| | ATSYADSVKGRFTISRDNAKNSLYLQMNSLRAEDTAVYYCARGRYYFDYWGQGTLV | |
| | TVSS | |
| 41 | EVQLVQSGGGLVHPGGSLRLSCAGSGFTFSRNAMFWVRQAPGKGLEWVSGIGTGG | SCB-VH-68 |
| | ATNYADSVKGRFTISRDNAKNSLYLQMNSLRAEDMAVYYCARGRYYFPWWGQGTL | |
| | VTVSS | |
| 42 | EVQLVESGGGLVHPGGSLRLSCAGSGFTFSRNAMFWVRQAPGKGLEWVSGIGTGG | SCB-VH-69 |
| | ATNYADSVKGRFTISRDNAKNSLYLQMNSLRAEDMAVYYCARGRYYFPWWGQGTL | |
| | VTVSS | |
| 43 | EVQLVQSGGGLVQPGGSLRLSCAGSGFTFSRNAMFWVRQAPGKGLEWVSGIGTG | SCB-VH-70 |
| | GATNYADSVKGRFTISRDNAKNSLYLQMNSLRAEDMAVYYCARGRYYFPWWGQG | |
| | TLVTVSS | |
| 44 | EVQLVQSGGGLVHPGGSLRLSCAASGFTFSRNAMFWVRQAPGKGLEWVSGIGTGG | SCB-VH-71 |
| | ATNYADSVKGRFTISRDNAKNSLYLQMNSLRAEDMAVYYCARGRYYFPWWGQGTL | |
| | VTVSS | |
| 45 | EVQLVQSGGGLVHPGGSLRLSCAGSGFTFSRNAMFWVRQAPGKGLEWVSGIGTGG | SCB-VH-72 |
| | ATSYADSVKGRFTISRDNAKNSLYLQMNSLRAEDMAVYYCARGRYYFPWWGQGTL | |
| | VTVSS | |
| 46 | EVQLVQSGGGLVHPGGSLRLSCAGSGFTFSRNAMFWVRQAPGKGLEWVSGIGTGG | SCB-VH-73 |
| | ATNYADSVKGRFTISRDNAKNSLYLQMNSLRAEDTAVYYCARGRYYFPWWGQGTL | |
| | VTVSS | |
| 47 | EVQLVQSGGGLVHPGRSLRLSCAGSGFTFSRNAMFWVRQAPGKGLEWVSGIGTGG | SCB-VH-74 |
| | ATNYADSVKGRFTISRDNAKNSLYLQMNSLRAEDMAVYYCARGRYYFDYWGQGTL | |
| | VTVSS | |
| 48 | EVQLVQSGGGLVHPGGSLRLTCAGSGFTFSRNAMFWVRQAPGKGLEWVSGIGTG | SCB-VH-75 |
| | GATNYADSVKGRFTISRDNAKNSLYLQMNSLRAEDMAVYYCARGRYYFDYWGQGT | |
| | LVTVSS | |
| 49 | EVQLVQSGGGLVHPGGSLRLSCAGSGFTFSRNAMHWVRQAPGKGLEWVSGIGTG | SCB-VH-76 |
| | GATNYADSVKGRFTISRDNAKNSLYLQMNSLRAEDMAVYYCARGRYYFDYWGQGT | |
| | LVTVSS | |
| 50 | EVQLVQSGGGLVHPGGSLRLSCAGSGFTFSRNAMFWVRQAPGEGLEWVSGIGTGG | SCB-VH-77 |
| | ATNYADSVKGRFTISRDNAKNSLYLQMNSLRAEDMAVYYCARGRYYFDYWGQGTL | |
| | VTVSS | |
| 51 | EVQLVQSGGGLVHPGGSLRLSCAGSGFTFSRNAMFWVRQAPGKGLEWVSGIGTGG | SCB-VH-78 |
| | ATNYADSVKGRFTISRDEAKNSLYLQMNSLRAEDMAVYYCARGRYYFDYWGQGTLV | |
| | TVSS | |
| 52 | EVQLVQSGGGLVHPGGSLRLSCAGSGFTFSRNAMFWVRQAPGKGLEWVSGIGTGG | SCB-VH-79 |
| | ATNYADSVKGRFTISRDNAKNSLYLQMNSLRAGDMAVYYCARGRYYFDYWGQGTL | |
| | VTVSS | |
| 53 | EVQLVQSGGGLVHPGGSLRLSCAGSGFTFDDYAMFWVRQAPGKGLEWVSGIGTG | SCB-VH-80 |
| | GATNYADSVKGRFTISRDNAKNSLYLQMNSLRAEDMAVYYCARGRYYFDYWGQGT | |
| | LVTVSS | |
| 54 | EVQLVQSGGGLVQPGGSLRLSCAASGFTFSRNAMFWVRQAPGKGLEWVSGIGTGG | SCB-VH-81 |
| | ATSYADSVKGRFTISRDNAKNSLYLQMNSLRAEDTAVYYCARGRYYFPWWGQGTLV | |
| | TVSS | |
| 55 | EVQLVESGGGLVHPGGSLRLSCAASGFTFSRNAMFWVRQAPGKGLEWVSGIGTGG | SCB-VH-82 |
| | ATSYADSVKGRFTISRDNAKNSLYLQMNSLRAEDTAVYYCARGRYYFPWWGQGTLV | |
| | TVSS | |
| 56 | EVQLVESGGGLVQPGGSLRLSCAGSGFTFSRNAMFWVRQAPGKGLEWVSGIGTGG | SCB-VH-83 |
| | ATSYADSVKGRFTISRDNAKNSLYLQMNSLRAEDTAVYYCARGRYYFPWWGQGTLV | |
| | TVSS | |
| 57 | EVQLVESGGGLVQPGGSLRLSCAASGFTFSRNAMFWVRQAPGKGLEWVSGIGTGG | SCB-VH-84 |
| | ATNYADSVKGRFTISRDNAKNSLYLQMNSLRAEDTAVYYCARGRYYFPWWGQGTL | |
| | VTVSS | |
| 58 | EVQLVESGGGLVQPGGSLRLSCAASGFTFSRNAMFWVRQAPGKGLEWVSGIGTGG | SCB-VH-85 |
| | ATSYADSVKGRFTISRDNAKNSLYLQMNSLRAEDMAVYYCARGRYYFPWWGQGTL | |
| | VTVSS | |
| 59 | EVQLVQSGGGLVHPGGSLRLSCAASGFTFSRNAMFWVRQAPGKGLEWVSGIGTGG | SCB-VH-86 |
| | ATSYADSVKGRFTISRDNAKNSLYLQMNSLRAEDTAVYYCARGRYYFPWWGQGTLV | |
| | TVSS | |
| 60 | EVQLVQSGGGLVQPGGSLRLSCAGSGFTFSRNAMFWVRQAPGKGLEWVSGIGTG | SCB-VH-87 |
| | GATSYADSVKGRFTISRDNAKNSLYLQMNSLRAEDTAVYYCARGRYYFPWWGQGTL | |
| | VTVSS | |
| 61 | EVQLVESGGGLVHPGGSLRLSCAGSGFTFSRNAMFWVRQAPGKGLEWVSGIGTGG | SCB-VH-88 |
| | ATSYADSVKGRFTISRDNAKNSLYLQMNSLRAEDTAVYYCARGRYYFPWWGQGTLV | |
| | TVSS | |
| 62 | EVQLVQSGGGLVHPGGSLRLSCAGSGFTFSRNAMFWVRQAPGKGLEWVSGIGTGG | SCB-VH-89 |
| | ATSYADSVKGRFTISRDNAKNSLYLQMNSLRAEDTAVYYCARGRYYFPWWGQGTLV | |
| | TVSS | |
| 63 | EVQLVESGGGLVQPGGSLRLSCAASGFTFSRNAMFWVRQAPGKGLEWVSGIGTGG | SCB-VH-90 |
| | ATNYADSVKGRFTISRDNAKNSLYLQMNSLRAEDMAVYYCARGRYYFPWWGQGTL | |
| | VTVSS | |
| 64 | EVQLVESGGGLVQPGGSLRLSCAASGFTFSRNAMFWVRQAPGKGLEWVSGIGTGG | SCB-VH-91 |
| | ATSYADSVKGRFTISRDNAKNSLYLQMNSLRAEDTAVYYCARGRYYFDYWGQGTLV | |
| | TVSS | |
| 65 | EVQLVQSGGGLVHPGGSLRLSCAGSGFTFSRNAMFWVRQAPGKGLEWVSGIGTGG | SCB-VH-92 |
| | ATNYADSVKGRFTISRDNAKNSLYLQMNSLRAEDMAVYYCARGRYYFDYWGQGTL | |
| | VTVSS | |
| 66 | EVQLVESGGGLVQPGGSLRLSCAASGFTFSRNAMFWVRQAPGKGLEWVSGIGTGG | SCB-VH-93 |
| | ATSYADSVKGRFTISRDNAKNSLYLQMNSLRAEDTAVYYCARGRYYFPWWGQGTLV | |
| | TVSS | |
| 67 | QVQLVQSGAEVKKPGASVKVSCKASGYAFTSYYMHWARQAPGQGLEWMGIINPS | MEDI-1-VH |
| | GGSTSYAQKFQGRVTMTRDTSTSTVYMELSSLRSEDTAVYYCARGKWWLDYWGKG | |
| | TLVTVSS | |
| 68 | QSVLTQPPSVSAAPGQKVTISCSGGSSNIGNSYVSWYQQLPGTAPKLLIYDNNKRPS | MEDI-1-VL |
| | GIPDRFSGSKSGTSATLAITGLQTGDEADYYCGTWDTSLSANYVFGTGTKLTVL | |
| 69 | QVQLVQSGAEVKKPGASVKVSCKASGYAFTSYYMHWARQAPGQGLEWMGIINPS | MEDI-2-VH |
| | GGSTSYAQKFQGRVTMTRDTSTSTVYMELSSLRSEDTAVYYCARGKWWLYNWGKG | |
| | TLVTVSS | |
| 70 | QSVLTQPPSVSAAPGQKVTISCSGGSSNIGNSYVSWYQQLPGTAPKLLIYDNNKRPS | MEDI-2-VL |
| | GIPDRFSGSKSGTSATLAITGLQTGDEADYYCGTWDTSQPPNPLFGTGTKLTVL | |
| 71 | QVQLVQSGAEVKKPGASVKVSCKASGYAFTSYYMHWARQAPGQGLEWMGIINPS | MEDI-3-VH |
| | GGSTSYAQKFQGRVTMTRDTSTSTVYMELSSLRSEDTAVYYCARGKLLKNPWGKGT | |
| | LVTVSS | |
| 72 | QSVLTQPPSVSAAPGQKVTISCSGGSSNIGNSYVSWYQQLPGTAPKLLIYDNNKRPS | MEDI-3-VL |
| | GIPDRFSGSKSGTSATLAITGLQTGDEADYYCGTWFGTPASNYVFGTGTKLTVL | |
| 73 | QVQLVQSGAEVKKPGASVKVSCKASGYAFTSYYMHWARQAPGQGLEWMGIINPS | MEDI-4-VH |
| | GGSTSYAQKFQGRVTMTRDTSTSTVYMELSSLRSEDTAVYYCARGKWWLYNWGKG | |
| | TLVTVSS | |
| 74 | QSVLTQPPSVSAAPGQKVTISCSGGSSNIGNSYVSWYQQLPGTAPKLLIYDNNKRPS | MEDI-4-VL |
| | GIPDRFSGSKSGTSATLAITGLQTGDEADYYCGTWDTSSPPQPIFGTGTKLTVL | |
| 75 | QVQLVQSGAEVKKPGASVKVSCKASGYAFTSYYMHWARQAPGQGLEWMGIINPS | MEDI-5-VH |
| | GGSTSYAQKFQGRVTMTRDTSTSTVYMELSSLRSEDTAVYYCARGKWWLYDWGKG | |
| | TLVTVSS | |
| 76 | QSVLTQPPSVSAAPGQKVTISCSGGSSNIGNSYVSWYQQLPGTAPKLLIYDNNKRPS | MEDI-5-VL |
| | GIPDRFSGSKSGTSATLAITGLQTGDEADYYCGTWDTSSPPQPIFGTGTKLTVL | |
| 77 | QVQLVQSGAEVKKPGASVKVSCKASGYAFTSYYMHWARQAPGQGLEWMGIINPS | MEDI-6-VH |
| | GGSTSYAQKFQGRVTMTRDTSTSTVYMELSSLRSEDTAVYYCARGKYWMYDWGKG | |
| | TLVTVSS | |
| 78 | QSVLTQPPSVSAAPGQKVTISCSGGSSNIGNSYVSWYQQLPGTAPKLLIYDNNKRPS | MEDI-6-VL |
| | GIPDRFSGSKSGTSATLAITGLQTGDEADYYCGTWDTSTTYHPIFGTGTKLTVL | |
| 79 | QVQLVQSGAEVKKPGASVKVSCKASGYAFTSYYMHWARQAPGQGLEWMGIINPS | MEDI-7-VH |
| | GGSTSYAQKFQGRVTMTRDTSTSTVYMELSSLRSEDTAVYYCARGKWWWQYWGK | |
| | GTLVTVSS | |
| 80 | QSVLTQPPSVSAAPGQKVTISCSGGSSNIGNSYVSWYQQLPGTAPKLLIYDNNKRPS | MEDI-7-VL |
| | GIPDRFSGSKSGTSATLAITGLQTGDEADYYCGTWDTSSPPQPIFGTGTKLTVL | |
| 81 | QVQLVQSGAEVKKPGASVKVSCKASGYAFTSYYMHWARQAPGQGLEWMGIINPS | MEDI-8-VH |
| | GGSTSYAQKFQGRVTMTRDTSTSTVYMELSSLRSEDTAVYYCARGKWWWQYWGK | |
| | GTLVTVSS | |
| 82 | QSVLTQPPSVSAAPGQKVTISCSGGSSNIGNSYVSWYQQLPGTAPKLLIYDNNKRPS | MEDI-8-VL |
| | GIPDRFSGSKSGTSATLAITGLQTGDEADYYCGTWDTSTTYHPIFGTGTKLTVL | |
| 83 | QVQLVQSGAEVKKPGASVKVSCKASGYAFTSYYMHWARQAPGQGLEWMGIINPS | MEDI-9-VH |
| | GGSTSYAQKFQGRVTMTRDTSTSTVYMELSSLRSEDTAVYYCARGKWWLYNWGKG | |
| | TLVTVSS | |
| 84 | QSVLTQPPSVSAAPGQKVTISCSGGSSNIGNSYVSWYQQLPGTAPKLLIYDNNKRPS | MEDI-9-VL |
| | GIPDRFSGSKSGTSATLAITGLQTGDEADYYCGTWDTSTTMYPLFGTGTKLTVL | |
| 85 | QVQLVQSGAEVKKPGASVKVSCKASGYAFTSYYMHWARQAPGQGLEWMGIINPS | MEDI-10-VH |
| | GGSTSYAQKFQGRVTMTRDTSTSTVYMELSSLRSEDTAVYYCARGKWWLYDWGKG | |
| | TLVTVSS | |
| 86 | QSVLTQPPSVSAAPGQKVTISCSGGSSNIGNSYVSWYQQLPGTAPKLLIYDNNKRPS | MEDI-10-VL |
| | GIPDRFSGSKSGTSATLAITGLQTGDEADYYCGTWDTSTVLTPIFGTGTKLTVL | |
| 87 | QVQLVQSGAEVKKPGASVKVSCKASGYAFTSYYMHWARQAPGQGLEWMGIINPS | MEDI-11-VH |
| | GGSTSYAQKFQGRVTMTRDTSTSTVYMELSSLRSEDTAVYYCARGKWWFYDWGKG | |
| | TLVTVSS | |
| 88 | QSVLTQPPSVSAAPGQKVTISCSGGSSNIGNSYVSWYQQLPGTAPKLLIYDNNKRPS | MEDI-11-VL |
| | GIPDRFSGSKSGTSATLAITGLQTGDEADYYCGTWDTSPSMIPLFGTGTKLTVL | |
| 89 | QVQLVQSGAEVKKPGASVKVSCKASGYAFTSYYMHWARQAPGQGLEWMGIINPS | MEDI-12-VH |
| | GGSTSYAQKFQGRVTMTRDTSTSTVYMELSSLRSEDTAVYYCARGKWWFYDWGKG | |
| | TLVTVSS | |
| 90 | QSVLTQPPSVSAAPGQKVTISCSGGSSNIGNSYVSWYQQLPGTAPKLLIYDNNKRPS | MEDI-12-VL |
| | GIPDRFSGSKSGTSATLAITGLQTGDEADYYCGTWDTSTTMYPLFGTGTKLTVL | |
| 91 | QVQLVQSGAEVKKPGASVKVSCKASGYAFTSYYMHWARQAPGQGLEWMGIINPS | MEDI-13-VH |
| | GGSTSYAQKFQGRVTMTRDTSTSTVYMELSSLRSEDTAVYYCARGKWWLYDWGKG | |
| | TLVTVSS | |
| 92 | QSVLTQPPSVSAAPGQKVTISCSGGSSNIGNSYVSWYQQLPGTAPKLLIYDNNKRPS | MEDI-13-VL |
| | GIPDRFSGSKSGTSATLAITGLQTGDEADYYCGTWDTSTTLQPLFGTGTKLTVL | |
| 93 | QVQLVQSGAEVKKPGASVKVSCKASGYAFTSYYMHWARQAPGQGLEWMGIINPS | MEDI-14-VH |
| | GGSTSYAQKFQGRVTMTRDTSTSTVYMELSSLRSEDTAVYYCARGKWWLYNWGKG | |
| | TLVTVSS | |
| 94 | QSVLTQPPSVSAAPGQKVTISCSGGSSNIGNSYVSWYQQLPGTAPKLLIYDNNKRPS | MEDI-14-VL |
| | GIPDRFSGSKSGTSATLAITGLQTGDEADYYCGTWDTSPPTKPLFGTGTKLTVL | |
| 95 | QVQLVQSGAEVKKPGASVKVSCKASGYAFTSYYMHWARQAPGQGLEWMGIINPS | MEDI-15-VH |
| | GGSTSYAQKFQGRVTMTRDTSTSTVYMELSSLRSEDTAVYYCARGKWWLYNWGKG | |
| | TLVTVSS | |
| 96 | QSVLTQPPSVSAAPGQKVTISCSGGSSNIGNSYVSWYQQLPGTAPKLLIYDNNKRPS | MEDI-15-VL |
| | GIPDRFSGSKSGTSATLAITGLQTGDEADYYCGTWDTSTHRHPLFGTGTKLTVL | |
| 97 | QVQLVQSGAEVKKPGASVKVSCKASGYAFTSYYMHWARQAPGQGLEWMGIINPS | MEDI-16-VH |
| | GGSTSYAQKFQGRVTMTRDTSTSTVYMELSSLRSEDTAVYYCARGKWWLYNWGKG | |
| | TLVTVSS | |
| 98 | QSVLTQPPSVSAAPGQKVTISCSGGSSNIGNSYVSWYQQLPGTAPKLLIYDNNKRPS | MEDI-16-VL |
| | GIPDRFSGSKSGTSATLAITGLQTGDEADYYCGTWDTSTTYHPIFGTGTKLTVL | |
| 99 | QVQLVQSGAEVKKPGASVKVSCKASGYAFTSYYMHWARQAPGQGLEWMGIINPS | MEDI-17-VH |
| | GGSTSYAQKFQGRVTMTRDTSTSTVYMELSSLRSEDTAVYYCARGKWWWQHWGK | |
| | GTLVTVSS | |
| 100 | QSVLTQPPSVSAAPGQKVTISCSGGSSNIGNSYVSWYQQLPGTAPKLLIYDNNKRPS | MEDI-17-VL |
| | GIPDRFSGSKSGTSATLAITGLQTGDEADYYCGTWDTSPVDRPIFGTGTKLTVL | |
| 101 | QVQLVQSGAEVKKPGASVKVSCKASGYAFTSYYMHWARQAPGQGLEWMGIINPS | MEDI-18-VH |
| | GGSTSYAQKFQGRVTMTRDTSTSTVYMELSSLRSEDTAVYYCARGKWWWQHWGK | |
| | GTLVTVSS | |
| 102 | QSVLTQPPSVSAAPGQKVTISCSGGSSNIGNSYVSWYQQLPGTAPKLLIYDNNKRPS | MEDI-18-VL |
| | GIPDRFSGSKSGTSATLAITGLQTGDEADYYCGTWDTSTTPMPVFGTGTKLTVL | |
| 103 | QVQLVQSGAEVKKPGASVKVSCKASGYAFTSYYMHWARQAPGQGLEWMGIINPS | MEDI-19-VH |
| | GGSTSYAQKFQGRVTMTRDTSTSTVYMELSSLRSEDTAVYYCARGKWWWQHWGK | |
| | GTLVTVSS | |
| 104 | QSVLTQPPSVSAAPGQKVTISCSGGSSNIGNSYVSWYQQLPGTAPKLLIYDNNKRPS | MEDI-19-VL |
| | GIPDRFSGSKSGTSATLAITGLQTGDEADYYCGTWDTSTTYHPIFGTGTKLTVL | |
| 105 | QVQLVQSGAEVKKPGASVKVSCKASGYAFTSYYMHWARQAPGQGLEWMGIINPS | MEDI-20-VH |
| | GGSTSYAQKFQGRVTMTRDTSTSTVYMELSSLRSEDTAVYYCARGKYWMYDWGKG | |
| | TLVTVSS | |
| 106 | QSVLTQPPSVSAAPGQKVTISCSGGSSNIGNSYVSWYQQLPGTAPKLLIYDNNKRPS | MEDI-20-VL |
| | GIPDRFSGSKSGTSATLAITGLQTGDEADYYCGTWDTSTVWEWPFGTGTKLTVL | |
| 107 | QVQLVQSGAEVKKPGASVKVSCKASGYAFTSYYMHWARQAPGQGLEWMGIINPS | MEDI-21-VH |
| | GGSASYAQKFQGRVTMTRDTSTSTVYMELSSLRSEDTAVYYCARGKYWMYDWGK | |
| | GTLVTVSS | |
| 108 | QSVLTQPPSVSAAPGQKVTISCSGGSSNIGNSYVSWYQQLPGTAPKLLIYDNNKRPS | MEDI-21-VL |
| | GIPDRFSGSKSGTSATLAITGLQTGDEAVYFCGTWDTSTVWEWPFGTGTKLTVL | |
| 109 | QVQLVQSGAEVKKPGASVKVSCKASGYAFTSYYMHWARQAPGQGLEWMGIINPS | MEDI-22-VH |
| | GGSTSYAQKFQGRVTMTRDTSTSTVYMELSSLRSEDTAVYYCARGKYWMYDWGKG | |
| | TLVTVSS | |
| 110 | QPVLTQPPSVSAAPGQKVTISCSGGSSNIGNSYVSWYQQLPGTAPKLLIYDNNKRPS | MEDI-22-VL |
| | GIPDRFSGSKSGTSATLAITGLQTGDEADYFCGTWDTSTVWEWPFGTGTKLTVL | |
| 111 | QVQLVQSGAEVRKPGASVKVSCKASGYAFTSYYMHWARQAPGQGLEWMGIINPS | MEDI-23-VH |
| | GGSTSYAQKFQGRVTMTRDTSTSTVYMELSSLRSEDTAVYYCARGKYWMYDWGKG | |
| | TLVTVSS | |
| 112 | QSVLTQPPSVSAAPGQKVTISCSGGSSNIGNNYVSWYQQLPGTAPKLLIYDNNKRPP | MEDI-23-VL |
| | GIPDRFSGSKSGTSATLAITGLQTGDEADYYCGTWDTSTVWEWPFGTGTKLTVL | |
| 113 | QVQLVQSGAEVKKPGASVKVSCKASGYAFTSYYMHWARQAPGQGLEWMGIINPR | MEDI-24-VH |
| | GGSTSYAQKFQGRVTMTRDTSTSTVYMELSSLRSEDTAVYYCARGKYWMYDWGKG | |
| | TLVTVSS | |
| 114 | QSVLTQPPSVSAAPGQKVTISCSGGSSNIGNSYVSWYQQLPGTAPKLLIYDNNKRPS | MEDI-24-VL |
| | GIPDRFSGSKSGTSATLAITGLQTGDEADYFCGTWDTSTVWEWPFGTGTKLTVL | |
| 115 | QVQLVQSGAEVKKPGASVKVSCKASGYAFTSYYMHWARQAPGQGLEWMGIINPR | MEDI-25-VH |
| | GGSASYAQKFQGRVSMTRDTSTSTVYMELSSLRSEDTAVYYCARGKYWMYDWGK | |
| | GTLVTVSS | |
| 116 | QSVLTQPPSVSAAPGQKVTISCSGGSSNIGNSYVSWYQQLPGTAPKLLIYDNNKRPS | MEDI-25-VL |
| | GIPDRFSGSKSGTTATLAITGLQTGDEADYYCGTWVTSTVWEWPFGTGTKLTVL | |
| 117 | QVQLVQSGAEVKKPGASVKVSCKASGYAFTSYYMHWARQAPGQGLEWMGIINPS | MEDI-26-VH |
| | GGSTSYAQKFQGRVTMTRDTSTSTVYMELSSLRSEDTAVYYCARGKYWMYDWGKG | |
| | TLVTVSS | |
| 118 | QSVLTQPPSVSAAPGQKVTISCSGGSSNIGNSYVSWYQQLPGTAPKLLIYDNNKRPS | MEDI-26-VL |
| | GIPDRFSGSKSGTSATLAITGLQTGDEADYFCGTWDTSTVWEWPFGTGTKLTVL | |
| 119 | QVQLVQSGAEVRKPGASVKVSCKASGYAFTSYYMHWARQAPGQGLEWMGIINPS | MEDI-27-VH |
| | GGSTSYAQKFQGRVTMTRDTSTSTVYMELSSLRPEDTAVYYCARGKYWMYDWGK | |
| | GTQVTVSS | |
| 120 | QSVLTQPPLVSAAPGQKVTISCSGGSSNIGNSYVSWYQRLPGTAPKLLIYDNNKRPSG | MEDI-27-VL |
| | IPDRFSGSKSGTSATLAITGLQTGDEADYYCGTWDTSTVWEWPFGTGTKLTVL | |
| 121 | QVQLVQSGAEVKKPGASVKVSCKASGYAFTSYYMHWARQAPGQGLEWMGIINPS | MEDI-28-VH |
| | GGSTSYAQKFQGRVTMTRDTSTSTVYMELSSLRSEDTAVYYCARGKYWMYDWGN | |
| | GTLVTVSS | |
| 122 | LPVLTQPPSVSAAPGQKVTISCSGGSSSIGNSYVSWYQQLPGAAPKLLIYDNNKRPSG | MEDI-28-VL |
| | IPDRFSGFRSGTSATLAITGLQTGDEADYYCGTWDTSPVWEWPFGTGTKLTVL | |
| 123 | QVQLVQSGAEVKKPGASVKVSCKASGYAFTSYYMHWARQAPGQGLEWMGIINPS | MEDI-29-VH |
| | GGSTSYAQKFQGRVTMTRDTSTSTVYMELSSLRSEDTAVYYCARGKYWMYDWGKG | |
| | TRVTVSS | |
| 124 | QSVLTQPPSVSAAPGQKVTISCSGGSSNIGNSYVSWYQQLPGTAPKLLIYDNNKRPS | MEDI-29-VL |
| | GIPDRFSGSKSGTSATLAITGLQTGDEADYYCGTWDTSPVWEWPFGTGTKLTVL | |
| 125 | QVQLVQSGAEVKKPGASVKVSCKASGYAFTSYYMHWARQAPGQGLEWMGIINPS | MEDI-30-VH |
| | GGSTSYAQKFQGRVTMTRDTSTSTVYMELSSLRSEDTAVYYCARGKYWMYDWGKG | |
| | TLVTVSS | |
| 126 | QSVLTQPPSVSAAPGQKVTISCSGGSSNIGNSYVSWYQRLPGAAPKLLIYDNNKRPS | MEDI-30-VL |
| | GIPDRFSGSKSGTSATLAITGLQTGDEADYYCGTWDTSTVWEWPFGTGTKLTVL | |
| 127 | QVQLVQSGAEVKKPGASVKVSCKASGYAFTSYYMHWARQAPGQGLEWMGIINPS | MEDI-31-VH |
| | GGSTSYAQKFQGRVTMTRDTSTSTVYMELSSLRSEDTAVYYCARGKYWMYDWGKG | |
| | TLVTVSS | |
| 128 | QSVLTQPPSVSAAPGQKVTISCSGGSSSIGNSYVSWYQQLPGTAPKLLIYDNNKRPSG | MEDI-31-VL |
| | IPDRFSGSKSGTSATLAITGLQTGDEADYYCGTWATSPVWEWPFGTGTKLTVL | |
| 129 | QVQLVQSGAEVKKPGASVKVSCKASGYAFTSYYMHWARQAPGQGLEWMGIINPS | MEDI-32-VH |
| | GGSTSYAQKFQGRVTMTRDTSTSTVYMELSSLRSEDTAVYYCARGKYWMYDWGKG | |
| | TLVTVSS | |
| 130 | QSVLTQPPSVSAAPGQKVTISCSGGSSNIGNSYVSWYQQLPGTAPKLLIYDNNKRPS | MEDI-32-VL |
| | GIPDRFSGSKSGTSATLAITGLQTGDEADYFCGTWDTSTAWEWPFGTGTKLTVL | |
| 131 | QVQLVQSGAEEKKPGASVKVSCKASGYAFTSYYMHWARQAPGQGLEWMGIINPS | MEDI-33-VH |
| | GGSTSYAQKFQGRVTMTRDTSTSTVYMELSSLRSEDTAVYYCARGKYWMYDWGKG | |
| | TLVTVSS | |
| 132 | QSALTQPPSVSAAPGQKVTISCSGGSSNIGNSYVSWYQQLPGTAPKLLIYDNNKRPS | MEDI-33-VL |
| | GIPDRFSGSKSGTSATLAITGLQTGDEADYFCGTWDTSTVWEWPFGTGTKLTVL | |
| 133 | QVQLVQSGAEVKKPGASVKVSCKASGYAFTSYYMHWARQAPGQGLEWMGIINPS | MEDI-34-VH |
| | GGSTSYAQKFQGRVSMTRDTSTSTVYMELSSLRSEDTAVYYCARGKYWMYDWGKG | |
| | TLVTVSS | |
| 134 | QSVLTQPPSVSAAPGQKVTISCSGGSSNIGNSYVSWYQQLPGTAPKLLIYDNNKRPS | MEDI-34-VL |
| | GIPDRFSGSKSGTSATLAITGLQTGDEADYFCGTWDTSTVWEWPFGTGTKLTVL | |
| 135 | QVQLVQSGAEVKKPGASVKVSCKASGYAFTSYYMHWARQAPGQGLEWMGIINPS | MEDI-35-VH |
| | GGSTSYAQKFQGRVTMTRDTSTSTVYMELSSLRSEDTAVYYCARGKYWMYDWGKG | |
| | TLVTVSS | |
| 136 | QSVLTQPPSVSAAPGQKVTISCSGGSSNIGNSYVSWYQQLPGTAPKLLIYDNNKRPS | MEDI-35-VL |
| | GIPDRFSGSKSGTSATLAITGLQTGDEADYYCGTWDTSPVWEWPFGTGTKLTVL | |
| 137 | QVQLVQSGAEVKKPGASVKVSCKASGYAFTSYYMHWARQAPGQGLEWMGIINPS | MEDI-36-VH |
| | GGSASYAQKFQGRVTMTRDTSTSTVYMELSSLRSEDTAVYYCARGKYWMYDWGK | |
| | GTLVTVSS | |
| 138 | QSVLTQPPSVSAAPGQKVTISCSGGSSNIGNSYVSWYQQLPGTAPKLLIYDNNKRPS | MEDI-36-VL |
| | GIPDRFSGSKSGTSATLAITGLQTGDEADYYCGTWDSSTVWEWPFGTGTKLTVL | |
| 139 | QVQLVQSGAEVKKPGASVKVSCKASGYAFTSYYMHWARQAPGQGLEWMGIINPR | MEDI-37-VH |
| | GGSTSYAQKFQGRVAMTRDTSTSTVYMELSSLRPEDTAVYYCARGKYWMYDWGK | |
| | GTLVTVSS | |
| 140 | QSVLTQPPSVSAAPGQKVTISCSGGGSSIGNSYVSWYQQLPGTAPKLLIYDNNKRPS | MEDI-37-VL |
| | GVPDRFSGSKSGTSATLAITGLQTGDEADYYCGTWDTSPVWEWPFGTGTKLTVL | |
| 141 | QVQLVQSGAEVKKPGASVKVSCKASGYAFTSYYMHWARQAPGQGLEWMGIINPS | MEDI-38-VH |
| | GGSASYAQKFQGRVTMTRDTSTSTVYMELSSLRSEDTAVYYCARGKYWMYDWGK | |
| | GTLVTVSS | |
| 142 | QSVLTQPPSVSAAPGQKVTISCSGGSSNIGNSYVSWYQQLPGTAPKLLIYDNNKRPS | MEDI-38-VL |
| | GIPDRFSGSKSGTSATLAITGLQTGDEADYFCGTWDTSTVWEWPFGTGTKLTVL | |
| 143 | QVQLVQSGAEVKKPGASVKVSCKASGYAFTSYYMHWARQAPGQGLEWMGIINPR | MEDI-39-VH |
| | GGSTSYAQKFQGRVTMTRDTSTSTVYMELSSLRSEDTAVYYCARGKYWMYDWGKG | |
| | TLVTVSS | |
| 144 | QSVLTQPPSVSAAPGQKVTISCSGGSSNIGNSYVSWYQQLPGTAPKLLIYDNNKRPS | MEDI-39-VL |
| | GIPDRFSGSKSGTSATLAITGLQTGDEADYYCGTWDTSTAWEWPFGTGTKLTVL | |
| 145 | QVQLVQSGAEVKKPGASVKVSCKASGYAFTSYYMHWARQAPGQGLEWMGIINPS | MEDI-40-VH |
| | GGSTSYAQKFQGRVTMTRDTSTSTVYMELSSLRSEDTAVYYCARGKYWMYDWGKG | |
| | TLVTVSS | |
| 146 | QSVLTQPPSVSAAPGQKVTISCSGGSSNIGNSYVSWYQQLPGTAPKLLIYDNNKRPS | MEDI-40-VL |
| | GIPDRFSGSKSGTSATLAITGLQTGDEADYYCGTWDSSTVWEWPFGTGTKLTVL | |
| 147 | QVQLVQSGAEVRKPGASVKVSCKASGYAFTSYYMHWARQAPGQGLEWMGIINPS | MEDI-41-VH |
| | GGSTSYAQKFQGRVTMTRDTSTSTVYMELSSLRPEDTAVYYCARGKYWMYDWGK | |
| | GTLVTVSG | |
| 148 | QSVLTQPPSVSAAPGQKVTISCSGGSTNIGNSYVSWYQRLPGTAPKLLIYDNNKRPP | MEDI-41-VL |
| | GIPDRFSGSKSGTSATLAITGLQTGDEADYYCGTWDTSTVWEWPFGTGTKLTVL | |
| 149 | QVQLVQSGAEVKKPGASVKVSCKASGYAFTSYYMHWARQAPGQGLEWVGIINPSG | MEDI-42-VH |
| | GSTSYAQKFQGRVTMTRDTSTSTVYMELSSLRSGDTAVYYCARGKYWMYDWGKGT | |
| | LVTVSS | |
| 150 | QAVLTQPPSVSAAPGQKVTISCSGGSSNIGNSYVSWYQRLPGAAPKLLIYDNNKRPS | MEDI-42-VL |
| | GIPDRFSGSKSGTSATLAITGLQTGDEADYYCGTWDTSTGWEWPFGTGTKLTVL | |
| 151 | QVQLVQSGAEVKKPGASVKVSCKASGYAFTSYYMHWVRQAPGQGLEWMGIINPR | MEDI-37GL-VH |
| | GGSTSYAQKFQGRVTMTRDTSTSTVYMELSSLRSEDTAVYYCARGKYWMYDWGKG | |
| | TLVTVSS | |
| 152 | QSVLTQPPSVSAAPGQKVTISCSGGGSSIGNSYVSWYQQLPGTAPKLLIYDNNKRPS | MEDI-37GL-VL |
| | GIPDRFSGSKSGTSATLGITGLQTGDEADYYCGTWDTSPVWEWPFGTGTKLTVL | |
| 153 | EVQLLESGGGLVQPGGSLRLSCAVSGFTFSNYAMSWVRQAPGKGLEWVSAISSGGG | AJOU-1-VH |
| | NIYYADSVKGRFTISRDNSKNTLYLQMNSLRAEDTAVYYCAKLRRYFDYWGQGTLVT | |
| | VSS | |
| 154 | EVQLLESGGGLVQPGGSLRLSCAASGFTFSDYAMSWVRQAPGKGLEWVSAISSGGS | AJOU-2-VH |
| | SIYYADSVKGRFTISRDNSKNTLHLQMNSLRAEDTAVYYCARGPQRSATAVFDYWG | |
| | QGTLVTVSS | |
| 155 | EVQLLESGGGLVQPGGSLRLSCAASGFTFSNYAMSWVRQAPGKGLEWVSWISPNS | AJOU-3-VH |
| | GNIYYADSVKGRFTISRDNSKNTLYLQMNSLRAEDTAVYYCARRPLSAAWSHSSYYN | |
| | AMDVWGQGTLVTVSS | |
| 156 | EVQLLESGGGLVQPGGSLRLSCAASGFTFSGYAMSWVRQAPGKGLEWVSLISHSGS | AJOU-4-VH |
| | NTYYADSVKGRFTISRDNSKNTLYLQMNSLRAEDTAVYYCARPHRAFDYWGQGTLV | |
| | TVSS | |
| 157 | EVQLLESGGGLVQPGGSLRLSCAASGFTFSNYAMSWVRQAPGKGLEWVSGISHGS | AJOU-5-VH |
| | GSIYYADSVKGRFTISRDNSKNTLYLQMNSLRAEDTAVYYCARPHRAFDYWGQGTLV | |
| | TVSS | |
| 158 | EVQLLESGGGLVQPGGSLRLSCAASGFTFSNYAMSWVRQAPGKGLEWVSGISHGN | AJOU-6-VH |
| | GSIYYADSVKGRFTISRDNSKNTLYLQMNSLRAEDTAVYYCAKTGRHFDYWGQGTLV | |
| | TVSS | |
| 159 | EVQLLESGGGLVQPGGSLRLSCAASGFTFSNYAMSWVRQAPGKGLEWVSSISPSGS | AJOU-7-VH |
| | SIYYADSVKGRFTISRDNSKNTLYLQMNSLRAEDTAVYYCARSYRAFDYWGQGTLVT | |
| | VSS | |
| 160 | EVQLLESGGGLVQPGGSLRLSCAASGFTFSNYAMSWVRQAPGKGLEWVSAISPSGG | AJOU-8-VH |
| | SIYYADSVKGRFTISRDNSKNTLYLQMNSLRAEDTAVYYCARAKRAFDYWGQGTLVT | |
| | VSS | |
| 161 | EVQLLESGGGLVQPGGSLRLSCAASGFTFSNYAMSWVRQAPGKGLEWVSAISPGSG | AJOU-9-VH |
| | STYYADSVKGRFTISRDNSKNTLYLQMNSLRAEDTAVYYCAKFRRHFDYWGQGTLVT | |
| | VSS | |
| 162 | EVQLLESGGGLVQPGGSLRLSCAASGFTFSNYAMSWVRQAPGKGLEWVSAISSGGG | AJOU-10-VH |
| | NIYYADSVKGRFTISRDNSKNTLYLQMNSLRAEDTAVYYCARVHRAFDYWGQGTLV | |
| | TVSS | |
| 163 | EVQLLESGGGLVQPGGSLRLSCAASGFTFSNYAMSWVRQAPGKGLEWVSAITSSGR | AJOU-69-VH |
| | SIYYADSVKGRFTISRDNSKNTLYLQMNSLRAEDTAVYYCARVHRAFDYWGQGTLVT | |
| | VSS | |
| 164 | EVQLLESGGGLVQPGGSLRLSCAASGFTFSNYAMSWVRQAPGKGLEWVSAITSSGA | AJOU-70-VH |
| | NIYYADSVKGRFTISRDNSKNTLYLQMNSLRAEDTAVYYCARVHRAFDYWGQGTLV | |
| | TVSS | |
| 165 | EVQLLESGGGLVQPGGSLRLSCAASGFTFSNYAMSWVRQAPGKGLEWVSAITSSGG | AJOU-71-VH |
| | NIYYADSVKGRFTISRDNSKNTLYLQMNSLRAEDTAVYYCARVHRAFDYWGQGTLV | |
| | TVSS | |
| 166 | EVQLLESGGGLVQPGGSLRLSCAASGFTFSNYAMSWVRQAPGKGLEWVSAITAGG | AJOU-72-VH |
| | GSIYYADSVKGRFTISRDNSKNTLYLQMNSLRAEDTAVYYCARVHRAFDYWGQGTLV | |
| | TVSS | |
| 167 | EVQLLESGGGLVQPGGSLRLSCAASGFTFSRHAMAWVRQAPGKGLEWVSAITSSGR | AJOU-83-VH |
| | SIYYADSVKGRFTISRDNSKNTLYLQMNSLRAEDTAVYYCARVHRAFDYWGQGTLVT | |
| | VSS | |
| 168 | QSVLTQPPSASGTPGQRVTISCSGSSSNIGNNYVNWYQQLPGTAPKLLIYDNSHRPS | AJOU-33-VL |
| | GVPDRFSGSKSGTSASLAISGLRSEDEADYYCGTWDASLSAYVFGGGTKLTVL | |
| 169 | QSVLTQPPSASGTPGQRVTISCSGSSSNIGNNNVSWYQQLPGTAPKLLIYANSKRPS | AJOU-34-VL |
| | GVPDRFSGSKSGTSASLAISGLRSEDEADYYCGSWDDSLSAYVFGGGTKLTVL | |
| 170 | QSVLTQPPSAPGTPGQRVTISCTGSSSNIGSNSVNWYQQLPGTAPKLLIYDDSHRPS | AJOU-35-VL |
| | GVPDRFSGSKSGTSASLAISGLRSEDEADYYCDAWDSSLSAYVFGGGTKLTVL | |
| 171 | QSVLTQPPSASGTPGQRVTLSCTGSSSNIGSNYVSWYQQLPGTAPKLLIYADSQRPSG | AJOU-36-VL |
| | VPDRFSGSKSGTSASLAISGLRSEDEADYYCGTWDDSLSGYVFGGGTKLTVL | |
| 172 | QSVLTQPPSASGTPGQRVTISCSSSSSNIGSNYVSWYQQLPGTAPKLLIYSDSHRPSG | AJOU-37-VL |
| | VPDRFSGSKSGTSASLAISGLRSEDEADYYCGSWDYSLSAYVFGGGTKLTVL | |
| 173 | QSVLTQPPSASGTPGQRVTISCTGSSSNIGNNTVSWYQQLPGTAPKLLIYDNSHRPS | AJOU-38-VL |
| | GVPDRFSGSKSGTSASLAISGLQSEDEADYYCGSWDYSLSAYVFGGGTKLTVL | |
| 174 | QSVLTQPPSASGTPGQRVTISCTGSSSNIGNNDVNWYQQLPGTAPKLLIYYDSQRPS | AJOU-39-VL |
| | GVPDRFSGSKSGTSASLAISGLRSEDEADYYCATWDASLSAYVFGGGTKLTVL | |
| 175 | QSVLTQPPSASGTPGQRVTISCSGSSSNIGSNAVNWYQQLPGTAPKLLIYYDNQRPS | AJOU-40-VL |
| | GVPDRFSGSKSGTSASLAISGLRSEDEADYYCGTWDDSLNGYVFGGGTKLTVL | |
| 176 | QSVLTQPPSASGTPGQRVTISCSGSSSNIGNNAVTWYQQLPGTAPKLLIYDDSHRPS | AJOU-41-VL |
| | GVPDRFSGSKSGTSASLAISGLRSEDEADYYCGSWDYSLSAYVFGGGTKLTVL | |
| 177 | QSVLTQPPSASGTPGQRVTISCSGSSSNIGSNTFNWYQQLPGTAPKLLIYADSHRPSG | AJOU-42-VL |
| | VPDRFSGSKSGTSASLAISGLRSEDEADYYCGTWDYSLSGYVLGGGTKLTVL | |
| 178 | QSVLTQPPSASGTPGQRVTISCSGSSSNIGSNTFNWYQQLPGTAPKLLIYADSHRPSG | AJOU-77-VL |
| | VPDRFSGSKSGTSASLAISGLRSEDEADYYCGTWDYSLSGYVLGGGTKLTVL | |
| 179 | QSVLTQPPSASGTPGQRVTISCSGSSSNIGSNTFNWYQQLPGTAPKLLIYADSHRPSG | AJOU-78-VL |
| | VPDRFSGSKSGTSASLAISGLRSEDEADYYCGTWDYSLRGYVLGGGTKLTVL | |
| 180 | QSVLTQPPSASGTPGQRVTISCSGSSSNIGSNTFNWYQQLPGTAPKLLIYADSHRPSG | AJOU-79-VL |
| | VPDRFSGSKSGTSASLAISGLRSEDEADYYCGYWDYSLSGYVLGGGTKLTVL | |
| 181 | QSVLTQPPSASGTPGQRVTISCSGSSSNIGSNTFNWYQQLPGTAPKLLIYADSHRPSG | AJOU-80-VL |
| | VPDRFSGSKSGTSASLAISGLRSEDEADYYCGTWDYSLSGYVLGGGTKLTVL | |
| 182 | QSVLTQPPSASGTPGQRVTISCSGSSANSRTDGFNWYQQLPGTAPKLLIYADSHRPS | AJOU-86-VL |
| | GVPDRFSGSKSGTSASLAISGLRSEDEADYYCGTWDYSLSGYVLGGGTKLTVLG | |
| 183 | QSVLTQPPSASGTPGQRVTISCSGSAQFGSRDNFNWYQQLPGTAPKLLIYADSHRPS | AJOU-87-VL |
| | GVPDRFSGSKSGTSASLAISGLRSEDEADYYCGTWDYSLSGYVLGGGTKLTVLG | |
| 184 | QSVLTQPPSASGTPGQRVTISCSGSTKQMHNYQFNWYQQLPGTAPKLLIYADSHRP | AJOU-88-VL |
| | SGVPDRFSGSKSGTSASLAISGLRSEDEADYYCGTWDYSLSGYVLGGGTKLTVLG | |
| 185 | QSVLTQPPSASGTPGQRVTISCSGSLLRGENLQFNWYQQLPGTAPKLLIYADSHRPS | AJOU-89-VL |
| | GVPDRFSGSKSGTSASLAISGLRSEDEADYYCGTWDYSLSGYVLGGGTKLTVLG | |
| 186 | QSVLTQPPSASGTPGQRVTISCSGSPLFPDSGSFNWYQQLPGTAPKLLIYADSHRPSG | AJOU-90-VL |
| | VPDRFSGSKSGTSASLAISGLRSEDEADYYCGTWDYSLSGYVLGGGTKLTVLG | |
| 187 | QSVLTQPPSASGTPGQRVTISCSGSAALDLSPSFNWYQQLPGTAPKLLIYADSHRPSG | AJOU-91-VL |
| | VPDRFSGSKSGTSASLAISGLRSEDEADYYCGTWDYSLSGYVLGGGTKLTVLG | |
| 188 | QVQLVQSGAEVKKPGASVKVSCKASGYTFTNYGISWVRQAPGQGLEWMGWISVY | REGN-VH-3 |
| | NGKTNYAQKLQGRVTMTTDTSTTTAYMEMRSLRSDDTAVYYCARGSGYDLDYWG | |
| | QGTLVSVSS | |
| 189 | EVQLVESGGGLVQPGGSLRLSCAASGFTFSSFWMTWVRQAPGKGLEWVANIKQD | REGN-VH-19 |
| | GSEKYYVDSVKGRFTISRDNAKNSLYLQMNSLRAEDTAVYYCARDPGRTMVRGGIRY | |
| | YYGMDVWGQGTTVTVSS | |
| 190 | EVKLAESGGGLVQPGGSLRLSCAASGFTFSSHWMNWVRQAPGKGLEWVANIKQD | REGN-VH-35 |
| | GSDKYYVDSVKGRFTISRDNAKNSLYLQLNSLIAEDTAVYYCARDRGVRPPRGAFDIW | |
| | GQGTMVTVSS | |
| 191 | QVQLVQSGAEVKKPGASVKVSCKASGYTFNSYGISWVRQAPGQGLEWMGWIRTY | REGN-VH-51 |
| | NGNTNYAQKLQGRVTMTTDTSTSTAYMELRSLRSDDTAVYYCARDEARIVVAGTTP | |
| | YYYGMDVWGQGTTVTVSS | |
| 192 | QVQLVESGGGLVQPGGSLRLSCAVSGFTISDHYMSWIRQAPGKGLEWISYISSSGSKI | REGN-VH-67 |
| | YYADSVKGRFTISRDNAKNSLFLQMNSLRAEDTAVYYCARTRQLVGDYWGQGTLVT | |
| | VSS | |
| 193 | EVQLVESGGGLVQPGRSLRLSCAASGFTFDNYAMHWVRQAPGKGLEWVSGIRWN | REGN-VH-83 |
| | SGSIGYADSVKGRFTISRDNAKNSLYLQMNSLRAEDTALYYCAKEGGYSGYRPGPFFD | |
| | YWGQGTLVTVSS | |
| 194 | QVQLVQSGAEVKKPGASVKVSCKASGYTFTNYGISWVRQAPGQGLEWMGWISVY | REGN-VH-99 |
| | NGHTNYAQKLQGRVTMTTDTSTSTAYMELRSLRSDDTAVYYCARGSGYDFDSWGQ | |
| | GTLVTVSS | |
| 195 | QVQLVQSGAEVKKPGASVKVSCKASRYTFTSYDINWVRQATGQGLEWMGWMNP | REGN-VH-115 |
| | NSGNTGYAQKFQGRVTMTRNTSTSTAYMELSSLRSEDTAVYYCARVRRFFDYWGQ | |
| | GTLVTVSS | |
| 196 | QVQLVQSGPEVKKPGASVKVSCKASGYTFTNYGISWVRQAPGQGLEWMGWISVY | REGN-VH-147 |
| | NGNINYAQKLQGRVTMTTDTSTSTAYMDLRSLRSDDTAVYYCARGSGYDFDYWGQ | |
| | GTLVTVSS | |
| 197 | QVQLVQSGAEVKKPGASVKVSCKDSAYTFNRYGISWVRQAPGQGLEWMGWISAY | REGN-VH-163 |
| | TGNTVYAQKLQGRVTMTTDNSTSTAYMELRSLRSDDTAVYYCARDKSIFGVVRGFD | |
| | YWGQGTLVTVSS | |
| 198 | AIQMTQSPSSLSASVGDRVTITCRASQGIRNALGWYQQKPGKAPKLLIYAASSLQSG | REGN-VL-11 |
| | VPSRFSGSGSGTDFTLTFSSLQPEDFATYYCLQDFNYPYTFGQGTKLEIK | |
| 199 | DIQMTQSPSSVSASVGDRVTISCRASQGVSSWLAWYQQKPGNAPKLLISAASSIQSG | REGN-VL-27 |
| | VPSRFSGSGSGTDFTLTISSLQPEDFATYYCQQANSFPLTFGGGTKVEIK | |
| 200 | DIQMTQSPSSVSASVGDRVTITCRASQGISSWLAWYQQKPGKAPKLLIYAASSFQSG | REGN-VL-43 |
| | VPSRFSGSGSGTDFTLTISSLQPEDFATYFCQQANSFPLTFGGGTTVEIK | |
| 201 | DIQMTQSPSSVSASVGDRVTITCRASQDISIWLAWYQQSPGKAPKLLINVASRLQSG | REGN-VL-59 |
| | VPSRFSGSGSGTDFTLTINSLQPEDFVTYYCQQANSFPITFGQGTRLATK | |
| 202 | DIQLTQSPSFLSASVGDRVTITCWASQGISSYLAWYQQKPGKAPKLLIFAASTLQSGV | REGN-VL-75 |
| | PSRFSGSGSGTEFTLTISSLQPEDFATYYCQQLNSYPLTFGGGTKVEIR | |
| 203 | EIVMTQSPATLSVSPGERATLSCRASQSVNYNLAWYQHKPGQAPRLLIYGASTRATGI | REGN-VL-91 |
| | PARFSGSGSGTEFTLTISSLQSEDFAVYYCQQYNNWPLTFGGGTKVEIK | |
| 204 | AIQMTQSSSSLSASVGDRVTITCRASQAIRNALGWYQQKPGKAPKVLIYAASSLQSGI | REGN-VL-107 |
| | PSRFSGSGSGTDFTLTISSLQPEDFATYYCLQDYDYPYTFGQGTKLEIK | |
| 205 | DIQLTQSPSFLSASVGDRVTITCWASQGIISYLAWYQQKPGKAPKLLIYAASTLHSGVP | REGN-VL-123 |
| | SRFSGSGSGTEFTLTISSLQPEDFATYYCHQLKSYPITFGQGTRLEIK | |
| 206 | AIQMTQSPSSLSASVGDRVTITCRASQDIRNALGWYQQKPGKAPKLLIYAASSLQSG | REGN-VL-155 |
| | VPSRFSGSASGTDFTLTISSLQPEDFAAYYCLQDYNYPYTFGQGTKLEIK | |
| 207 | EIVMTQSPVTLSLSPGERATLPCRASQSVSSSLAWYQQKAGQSPRLLIYGASTRATGI | REGN-VL-171 |
| | PARFSGSGSGTEFTLTISNLQSEDFAVYYCQQYNNWPLTFGGGTKVEIK | |
| 208 | EVQLLESGGGLVQPGGSLRLSCAASGFTLSSYAMHWVRQAPGKGLEYVSGISSNGG | STSA-C27-VH |
| | STYYANSVKGRFTISRDNPKNTLFLQMSSLRAEDTAVYYCVRVKVGYRGGMDVWG | |
| | QGTTVTVSS | |
| 209 | EVQLLESGGGLVQPGGSLRLSCAASGFTLSSYAMHWVRQAPGKGLEYVSGISPSGSS | STSA-C27-6-33-VH |
| | TYYANSVKGRFTISRDNPKNTLFLQMSSLRAEDTAVYYCVRSKVRYRGGMDVWGQ | |
| | GTTVTVSS | |
| 210 | EVQLLESGGGLVQPGGSLRLSCAASGFTLSSYAMHWVRQAPGKGLEYVSGISPSGVS | STSA-C27-7-33-VH |
| | TYYANSVKGRFTISRDNPKNTLFLQMSSLRAEDTAVYYCVRVKVKYRGGMDVWGQ | |
| | GTTVTVSS | |
| 211 | EVQLLESGGGLVQPGGSLRLSCAASGFTLSSYAMHWVRQAPGKGLEYVSGISPTSGS | STSA-C27-24-56-VH |
| | TYYANSVKGRFTISRDNPKNTLFLQMSSLRAEDTAVYYCVRVKVRYRGGMDVWGQ | |
| | GTTVTVSS | |
| 212 | EVQLLESGGGLVQPGGSLRLSCAASGFTLSSYAMHWVRQAPGKGLEYVSGISPTGTS | STSA-C27-47-56-VH |
| | TYYANSVKGRFTISRDNPKNTLFLQMSSLRAEDTAVYYCVRVKGAYRGGMDVWGQ | |
| | GTTVTVSS | |
| 213 | EVQLLESGGGLVQPGGSLRLSCAASGFTLSSYAMHWVRQAPGKGLEYVSGISSSGSS | STSA-C27-33-33-VH |
| | TYYANSVKGRFTISRDNPKNTLFLQMSSLRAEDTAVYYCVRVKVAYRGGMDVWGQ | |
| | GTTVTVSS | |
| 214 | EVQLLESGGGLVQPGGSLRLSCAASGFTLSSYAMHWVRQAPGKGLEYVSGISPSSTS | STSA-C27-56-56-VH |
| | TYYANSVKGRFTISRDNPKNTLFLQMSSLRAEDTAVYYCVRVKVLYRGGMDVWGQ | |
| | GTTVTVSS | |
| 215 | EVQLLESGGGLVQPGGSLRLSCAASGFTLSSYAMHWVRQAPGKGLEYVSGISPSSAS | STSA-C27-78-78-VH |
| | TYYANSVKGRFTISRDNPKNTLFLQMSSLRAEDTAVYYCVRVKSKYRGGMDVWGQ | |
| | GTTVTVSS | |
| 216 | EVQLLESGGGLVQPGGSLRLSCAASGFTLSSYAMHWVRQAPGKGLEYVSGISGNSAS | STSA-C27-82-58-VH |
| | TYYANSVKGRFTISRDNPKNTLFLQMSSLRAEDTAVYYCVRVKLKYRGGMDVWGQG | |
| | TTVTVSS | |
| 217 | EVQLLESGGGLVQPGGSLRLSCAASGFTLSSYAMHWVRQAPGKGLEYVSGISHSGTS | STSA-C27-54-54-VH |
| | TYYANSVKGRFTISRDNPKNTLFLQMSSLRAEDTAVYYCVRVRVLYRGGMDVWGQ | |
| | GTTVTVSS | |
| 218 | EVQLLESGGGLVQPGGSLRLSCAASGFTLSSYAMHWVRQAPGKGLEYVSGISPSGVS | STSA-C27-36-36-VH |
| | TYYANSVKGRFTISRDNPKNTLFLQMSSLRAEDTAVYYCVRVKVKYRGGMDVWGQ | |
| | GTTVTVSS | |
| 219 | EVQLLESGGGLVQPGGSLRLSCAASGFTLSSYAMHWVRQAPGKGLEYVSGISSNGG | STSA-C27-53-53-VH |
| | STYYANSVKGRFTISRDNPKNTLFLQMSSLRAEDTAVYYCVRVFVRYRGGMDVWGQ | |
| | GTTVTVSS | |
| 220 | EVQLLESGGGLVQPGGSLRLSCAASGFTLSSYAMHWVRQAPGKGLEYVSGISPTSAS | STSA-C27-67-67-VH |
| | TYYANSVKGRFTISRDNPKNTLFLQMSSLRAEDTAVYYCVRVKGRYRGGMDVWGQ | |
| | GTTVTVSS | |
| 221 | EVQLLESGGGLVQPGGSLRLSCAASGFTLSSYAMHWVRQAPGKGLEYVSGISPTGGS | STSA-C27-55-55-VH |
| | TYYANSVKGRFTISRDNPKNTLFLQMSSLRAEDTAVYYCVRVKGRYRGGMDVWGQ | |
| | GTTVTVSS | |
| 222 | EVQLLESGGGLVQPGGSLRLSCAASGFTLSSYAMHWVRQAPGKGLEYVSGISHSGN | STSA-C27-59-59-VH |
| | STYYANSVKGRFTISRDNPKNTLFLQMSSLRAEDTAVYYCVRVKRRYRGGMDVWGQ | |
| | GTTVTVSS | |
| 223 | EVQLLESGGGLVQPGGSLRLSCAASGFTLSSYAMHWVRQAPGKGLEYVSGISPSSNS | STSA-C27-58-58-VH |
| | TYYANSVKGRFTISRDNPKNTLFLQMSSLRAEDTAVYYCVRVKVRYRGGMDVWGQ | |
| | GTTVTVSS | |
| 224 | EVQLLESGGGLVQPGGSLRLSCAASGFTLSSYAMHWVRQAPGKGLEYVSGISSSGSS | STSA-C27-52-52-VH |
| | TYYANSVKGRFTISRDNPKNTLFLQMSSLRAEDTAVYYCVRVKPAYRGGMDVWGQ | |
| | GTTVTVSS | |
| 225 | EVQLLESGGGLVQPGGSLRLSCAASGFTLSSYAMHWVRQAPGKGLEYVSGISYSSAS | STSA-C27-Y2-Y2-VH |
| | TYYANSVKGRFTISRDNPKNTLFLQMSSLRAEDTAVYYCVRVKVRYRGGMDVWGQ | |
| | GTTVTVSS | |
| 226 | ETTLTQSPDTLPLSPGDRASLSCRASQSVSSAYLAWYQQKPGQAPRLLIYGTSRRATG | STSA-C27-VL |
| | VPGRFSGSGSGTDFTLTISRLEPEDFAVYYCQLYGSSSVTFGQGTKLEIK | |
| 227 | EIVLTQSPGTLSLSPGERATLSCRASQGISSAYLAWYQQKPGQAPRLLIYGTSRRATGI | STSA-C27-6-33-VL |
| | PDRFSGSGSGTDFTLTISRLEPEDFAVYYCQLYGATSVTFGQGTKLEIK | |
| 228 | EIVLTQSPGTLSLSPGERATLSCRASQGISSAYLAWYQQKPGQAPRLLIYGTSRRATGI | STSA-C27-7-33-VL |
| | PDRFSGSGSGTDFTLTISRLEPEDFAVYYCQLYGATSVTFGQGTKLEIK | |
| 229 | EIVLTQSPGTLSLSPGERATLSCRASQSVSSAYLAWYQQKPGQAPRLLIYGTSRRATGI | STSA-C27-24-56-VL |
| | PDRFSGSGSGTDFTLTISRLEPEDFAVYYCQLYGASSVTFGQGTKLEIK | |
| 230 | EIVLTQSPGTLSLSPGERATLSCRASQSVSSAYLAWYQQKPGQAPRLLIYGTSRRATGI | STSA-C27-47-56-VL |
| | PDRFSGSGSGTDFTLTISRLEPEDFAVYYCQLYGASSVTFGQGTKLEIK | |
| 231 | EIVLTQSPGTLSLSPGERATLSCRASQGISSAYLAWYQQKPGQAPRLLIYGTSRRATGI | STSA-C27-33-33-VL |
| | PDRFSGSGSGTDFTLTISRLEPEDFAVYYCQLYGATSVTFGQGTKLEIK | |
| 232 | EIVLTQSPGTLSLSPGERATLSCRASQSVSSAYLAWYQQKPGQAPRLLIYGTSRRATGI | STSA-C27-56-56-VL |
| | PDRFSGSGSGTDFTLTISRLEPEDFAVYYCQLYGASSVTFGQGTKLEIK | |
| 233 | EIVLTQSPGTLSLSPGERATLSCRASQSISTAYLAWYQQKPGQAPRLLIYGTSRRATGIP | STSA-C27-78-78-VL |
| | DRFSGSGSGTDFTLTISRLEPEDFAVYYCQLYGASSVTFGQGTKLEIK | |
| 234 | EIVLTQSPGTLSLSPGERATLSCRASQDISSAYLAWYQQKPGQAPRLLIYGTSRRATGI | STSA-C27-82-58-VL |
| | PDRFSGSGSGTDFTLTISRLEPEDFAVYYCQLYGATSVTFGQGTKLEIK | |
| 235 | EIVLTQSPGTLSLSPGERATLSCRASQDVSSAYLAWYQQKPGQAPRLLIYGTSRRATGI | STSA-C27-54-54-VL |
| | PDRFSGSGSGTDFTLTISRLEPEDFAVYYCQLYGATSVTFGQGTKLEIK | |
| 236 | EIVLTQSPGTLSLSPGERATLSCRASQNISTAYLAWYQQKPGQAPRLLIYGTSRRATGI | STSA-C27-36-36-VL |
| | PDRFSGSGSGTDFTLTISRLEPEDFAVYYCQLYGATSVTFGQGTKLEIK | |
| 237 | EIVLTQSPGTLSLSPGERATLSCRASQDASNAYLAWYQQKPGQAPRLLIYGTSRRATG | STSA-C27-53-53-VL |
| | IPDRFSGSGSGTDFTLTISRLEPEDFAVYYCQLYGSSSVTFGQGTKLEIK | |
| 238 | EIVLTQSPGTLSLSPGERATLSCRASQGVSSAYLAWYQQKPGQAPRLLIYGTSRRATGI | STSA-C27-67-67-VL |
| | PDRFSGSGSGTDFTLTISRLEPEDFAVYYCQLYGRSSVTFGQGTKLEIK | |
| 239 | EIVLTQSPGTLSLSPGERATLSCRASQNISTAYLAWYQQKPGQAPRLLIYGTSRRATGI | STSA-C27-55-55-VL |
| | PDRFSGSGSGTDFTLTISRLEPEDFAVYYCQLYGTSSVTFGQGTKLEIK | |
| 240 | EIVLTQSPGTLSLSPGERATLSCRASQSVSTAYLAWYQQKPGQAPRLLIYGTSRRATGI | STSA-C27-59-59-VL |
| | PDRFSGSGSGTDFTLTISRLEPEDFAVYYCQLYGATSVTFGQGTKLEIK | |
| 241 | EIVLTQSPGTLSLSPGERATLSCRASQDISSAYLAWYQQKPGQAPRLLIYGTSRRATGI | STSA-C27-58-58-VL |
| | PDRFSGSGSGTDFTLTISRLEPEDFAVYYCQLYGATSVTFGQGTKLEIK | |
| 242 | EIVLTQSPGTLSLSPGERATLSCRASQGVSTAYLAWYQQKPGQAPRLLIYGTSRRATGI | STSA-C27-52-52-VL |
| | PDRFSGSGSGTDFTLTISRLEPEDFAVYYCQLYGATSVTFGQGTKLEIK | |
| 243 | EIVLPQSPGTLSLSPGERATLSCRASQGVSSAYLAWYQQKPGQAPRLLIYGTSRRATGI | STSA-C27-Y2-Y2-VL |
| | PDRFSGSGSGTDFTLTISRLEPEDFAVYYCQLYGSTSVTFGQGTKLEIK | |
| 244 | EVQLVESGGGLVQPKGSLKLSCAASGFTFNTYGMHWVRQAPGKGLEWVAHIRSKS | Y0188-1 VH |
| | SNYATYYADSVKDRFTISRDDSQSMLYLQMNNLKTEDTAMYYCVRWFRAMDYWG | |
| | QGTSVTVSS | |
| 245 | EVQLIESGGGLVQPKGSLKLSCAASGFTFNMYAMDWVRQAPGKGLEWVARIRSKG | Y0188-2 VH |
| | SNFETNYADSVKDRFTISRDDSQSMVYLQMINLKTEDTAMYYCVRHRGGAWFAYW | |
| | GQGTLVSVSA | |
| 246 | QVQLVETGGGLVRPGNSLKLSCVTSGFTFSNYRMHWLRQPPGKRLEWIAVITVKSN | Y0188-3 VH |
| | NYGANYAESVKGRFAISRDDSKSSVYLEMNRLREEDTATYFCSRERAYGNPFDYWG | |
| | QGTTLTVSS | |
| 247 | EVQLVESGGGLVQPKGSLKLSCAASGFTFNMYAMNWVRQAPGQGLEWVARIRSKS | Y0188-4 VH |
| | NNYATYYADSVKDRFIISRDDSESMVYLQMSNLRAADTAMYYCVRHLRAMDYWG | |
| | QGTSVTVSS | |
| 248 | EVQLVESGGGLVQPKGSLKLSCAASGFSFNMYAMNWVRQAPGKGLEWVARIRTKS | Y0188-6 VH |
| | NHYSTYYADSVKDRFTISRDDSASMFYLQMNNLKTEDTAMYFCVRHLRAMDYWG | |
| | QGTSVTVSS | |
| 249 | EVQLIESGGGLVQPKGSLKLSCAASGFTFNMYAMDWVRQAPGKGLEWVARIRSKG | Y0188-8 VH |
| | SNFETNYADSVKDRFTISRDDSQSMVYLQMNNLKTEDTAMYYCVRHRGGAWFAY | |
| | WGQGTLVTVSA | |
| 250 | EVQLVESGGGLVRPKGSLKLSCAASGFSFNTYAMNWVRQAPGKGLEWIVWIRSKSH | Y0188-9 VH |
| | NYATYYADSVKDRFTISRDDSESMLYLQMNNLKTEDTAMYYCVRHLRAMDYWGQ | |
| | GTSVTVSS | |
| 251 | EVRLVESGGGLVQPKGSLKLSCEASGFSFNMYAMNWVRQAPGKGLEWITHIRSKSN | Y0188-10 VH |
| | NYATYYADSVKDRFIISRDDSESMVYLQMNNLKTEDTAMYYCVRLLRALDYWGQGT | |
| | SVTVSS | |
| 252 | EVQLVESGGGLVQPKGSLKLSCAASGFTFNMYGMHWVRQAPGKGLEWVAHIRSKS | Y0188-14 VH |
| | SNYATYYADSVKDRLTISRDDSQSMLYLQMNNLKTEDTAMYYCVRWFRAMDYWG | |
| | QGTSVTVSS | |
| 253 | EVQLVESGGGLVKPGGSLRLSCAASGFTFSMYGMHWVRQAPGKGLEWVGHIRSKS | HV3-15-14 VH |
| | SNYATYYADSVKDRFTISRDDSKNTLYLQMNSLKTEDTAVYYCTTWFRAMDYWGQ | |
| | GTLVTVSS | |
| 254 | EVQLVESGGGLVQPGGSLRLSCAASGFTFSMYGMHWVRQAPGKGLEWVSHIRSKS | HV3-48-14 VH |
| | SNYATYYADSVKDRFTISRDNAKNSLYLQMNSLRAEDTAVYYCARWFRAMDYWGQ | |
| | GTLVTVSS | |
| 255 | EVQLVESGGGLVQPGGSLKLSCAASGFTFSMYGMHWVRQASGKGLEWVGHIRSKS | HV3-73*2-14 VH |
| | SNYATYYADSVKDRFTISRDDSKNTAYLQMNSLKTEDTAVYYCTRWFRAMDYWGQ | |
| | GTLVTVSS | |
| 256 | EVQLVESGGGLVQPGGSLRLSCAASGFTFSMYGMHWVRQAPGKGLEWVGHIRSKS | HV3-72-14 VH |
| | SNYATYYADSVKDRFTISRDDSKNSLYLQMNSLKTEDTAVYYCARWFRAMDYWGQ | |
| | GTLVTVSS | |
| 257 | EVQLVESGGGLVQPGGSLRLSCAASGFTFSMYGMHWVRQAPGKGLEWVSHIRSKS | Y01-14 VH |
| | SNYATYYADSVKDRFTISRDNAKNSLYLQMNSLRAEDTAVYYCARWFRAMDYWGQ | |
| | GTLVTVSS | |
| 258 | EVQLVESGGGLEQPGGSLRLSCAGSGFTFRMYGMHWVRQAPGKGLEWVSHIRSKS | 162-14 VH |
| | SNYATYYADSVKDRFTISRDNSKNTLYLQMNSLRAEDTAVYYCAKWFRAMDYWGQ | |
| | GTTVTVSS | |
| 259 | EVQLVESGGGLVQPGGSLKLSCAASGFTFSMYGMHWVRQASGKGLEWVGHIRSKS | VH73-14 VH |
| | SNYATYYADSVKDRFTISRDDSKNTAYLQMNSLKTEDTAVYYCTRWFRAMDYWGQ | |
| | GTTVTVSS | |
| 260 | DIVMTQSHKFMSTSVGDRVSITCKASQDVSTAVAWYQEKPGQSPKLLIYWASTRHT | Y0188-1 VL |
| | GVPDRFTGSGSGTDYTLTISSVQAEDLALYYCQQHYSTPLTFGAGTKLELK | |
| 261 | DIVVTQSPASLAVSLGQRATISCRASKSVSTSGYSYMHWYQQKPGQPPKLLIYLASNL | Y0188-2 VL |
| | ESGVPARFSGSGSGTDFTLNIHPVEEEDVAIYYCQHSRELPLTFGAGTKLELK | |
| 262 | DIQMTQSPSSLSASLGERVSLTCRASQEISGYLSWLQQKPDGTIKRLIYAASTLDSGVP | Y0188-3 VL |
| | KRFSGSRSGSDYSLTISSLESEDFADYYCLQYGSYPYTFGGGTKLEIK | |
| 263 | DIVLTQSPASLTVSLGQRATISCRASKSVSTSGYSYMHWYQQKPGQPPKLLIYLASNLE | Y0188-4 VL |
| | SGVPARFSGSGSGTDFTLNIHPVEEEDAATYYCQHSRELPITFGSGTKLEIK | |
| 264 | DIVLTQSPASLVVSLGQRATISCRASQSVSTSGYSYMHWYQQKPGQPPKLLIYLASNV | Y0188-6 VL |
| | QSGVPARFSGSGSGTDFTLNIHPVEEEDVATYYCHHNRDLPFTFGSGTKLEIK | |
| 265 | DIVVTQSPASLAVSLGQRATISCRASKSVSTSGYSYMHWYQQKPGQPPKLLIYLASNL | Y0188-8 VL |
| | ESGVPARFSGSGSGTDFTLNIHPVEEEDVAIYYCQHSRELPLTFGAGTKLELK | |
| 266 | DIVLTQSPASLAVSLGQRATISCRASKSVSASGYSYMHWYQQKPGQPPKLLIYLASNL | Y0188-9 VL |
| | QSGVPARFSGSGSGTDFTLNIHPVEEEDAATYYCQHSRELPPTFGGGTKLEIK | |
| 267 | DIVLTQSPASLAVFLGQRATISCRASKSVSTSGYSYMHWYQQKAGQPPKLLIYLASNL | Y0188-10 VL |
| | ESGVPARFSGSGSGTDFTLNIHPVEEEDAATYYCHHSRELPITFGSGTKLEMK | |
| 268 | DIVMTQSHKFMSTSVGDRVSITCKASQDVSTAVAWYQEKPGQSPKLLIYWASTRHT | Y0188-14 VL |
| | GVPDRFTGSGSGTDYTLTISSVQAEDLALYYCQQHYSTPLTFGAGTKLELK | |
| 269 | EIVLTQSPGTLSLSPGERATLSCKASQDVSTAVAWYQQKPGQAPRLLIYWASTRHTGI | Y01-14 VL |
| | PDRFSGSGSGTDFTLTISRLEPEDFAVYYCQQHYSTPLTFGQGTKVEIK | |
| 270 | DIVMTQSPLSLPVTPGEPASISCKASQDVSTAVAWYLQKSGQSPQLLIYWASTRHTG | 164-14 VL |
| | VPDRFSGSGSGTDFTLKISRVEAEDVGFYYCQQHYSTPLTFGQGTKLEIK | |
| 271 | DIVMTQSPDSLAVSLGERATINCKASQDVSTAVAWYQQKPGQPPKLLIYWASTRHT | KV4-14 VL |
| | GVPDRFSGSGSGTDFTLTISSLQAEDVAVYYCQQHYSTPLTFGGGTKVEIK | |
| 272 | DIQMTQSPSSLSASVGDRVTITCKASQDVSTAVAWYQQKPGKVPKLLIYWASTRHT | KV1-27-14 VL |
| | GVPSRFSGSGSGTDFTLTISSLQPEDVATYYCQQHYSTPLTFGGGTKVEIK | |
| 273 | DIQLTQSPSFLSASVGDRVTITCKASQDVSTAVAWYQQKPGKAPKLLIYWASTRHTG | KV1-9-14 VL |
| | VPSRFSGSGSGTEFTLTISSLQPEDFATYYCQQHYSTPLTFGGGTKVEIK | |
| 274 | DIQMTQSPSSLSASVGDRVTITCKASQDVSTAVAWYQQKPGKAPKLLLYWASTRHT | KV1-NL1-14 VL |
| | GVPSRFSGSGSGTDYTLTISSLQPEDFATYYCQQHYSTPLTFGGGTKVEIK | |
| 275 | AIRMTQSPFSLSASVGDRVTITCKASQDVSTAVAWYQQKPAKAPKLFIYWASTRHTG | KV1D-43-14 VL |
| | VPSRFSGSGSGTDYTLTISSLQPEDFATYYCQQHYSTPLTFGGGTKVEIK | |
| 276 | QVQLQQSGTELVKPGASVKMSCKASVNTFTGYNMHWIKQTPGQGLEWIG | 1A6 VH |
| | GLHPGNGDSSYNQKFKGRATLTDKSSNTAYMQLSSLTSEDSAVYYCAL | |
| | TTAGRAWFPYWGQGTLVTVSA | |
| 277 | DIQMTQTTSSLSASLGDRVTISCRASQDISNYLNWYQQKPDGTVKLLIYYTSRLHSGV | 1A6 VL |
| | PSRFSGSGSGTDYSLTISNLEQEDIATYFCQQGNTLPPTFGGGTKLEIK | |
| 278 | QVQLQQSGTELVKPGASVKMSCKASVNTFTGYNMHWIKQTPGQGLEWIG | 1D8 VH |
| | GLHPGNGDSSYNQKFKGRATLTADKSSNTAYMQLSSLTSEDSAVYYCALTTAGRAW | |
| | FAYWGQGTLVTVSA | |
| 279 | DIQMTQTTSSLSASLGDRVTISCRASQDISNYLNWYQQKPDGTVKLLIYYTSRLHSGV | 1D8 VL |
| | PSRFSGSGSGTDYSLTISNLEQEDIATYFCQQGNTLHTFGGGTKLEIK | |
| 280 | QVQLQQSGAELVKPGASVKMSCKASVNTFAGYNMHWVKQTPGQGLEWIGGLHP | 1H9 VH |
| | GNGDTSYNQKFKGKATLTADKSSNTAYMQLSSLTSEDSAVYYCALTTAGRAWFAYW | |
| | GQGTLVTVSA | |
| 281 | DIQMIQSTSSLSASLGDRVTISCRASQDISNYLNWYQQKPDGTVKLLIYYTSRLHSGVP | 1H9 VL |
| | SRFSGSGSGTDYSLTISNLEQEDIATYFCQQGNTLPWTFGGGTKLEIK | |
| 282 | QVQLQQSGAELVKPGASVKMSCKASVNTFTGYNMHWVKQTPGQGLEWIGGLHP | 2H1 VH |
| | GNGDTSYNQKFKGKATLTADKSSNTAYMQLSSLTSEDSAVYYCALTTAGRAWFAYW | |
| | GQGTLVTVSA | |
| 283 | DIQMIQSTSSLSASLGDRVTISCRASQDISNYLNWYQQKPDGTVKLLIYYTSRLHSGVP | 2H1 VL |
| | SRFSGSGSGTDYSLTISNLEQEDIATYFCQQGNTLPLTFGAGTKLELK | |
| 284 | QVQLQQSGAELVKPGASVKMSCKASVNTFTGYNMHWVKQTPGQGLEWIGGLHP | 2F8 VH |
| | GNGDTSYNQKFKGKATLTADKSSNTAYMQLSSLTSEDSAVYYCALTTAGRAWFPYW | |
| | GQGTLVTVSA | |
| 285 | DIQMIQSTSSLSASLGDRVTISCRASQDISNYLNWYQQKPDGTVKLLIYYTSRLHSGVP | 2F8 VL |
| | SRFSGSGSGTDYSLTISNLEQEDIATYFCQQGNTLPWTFGGGTKLELK | |
| 286 | QVQLQQSGAELVKPGASVKMSCKASVNTFTGYNMHWIKQTPGQGLEWIGGLHPG | 9B4 VH |
| | NGDTSYNQKFKGKATLTADKSSNTAYMQLSSLTSEDSAVYYCALTTAGRAWFAYWG | |
| | QGTLVTVSA | |
| 287 | DIQMIQSTSSLSASLGDRVTISCRASQDISNYLNWYQQKPDGTVKLLIYYTSRLHSGVP | 9 B4 VL |
| | SRFSGSGSGTDYSLTISNLEQEDIATYFCQQGNTLPWTFGGGTKLELK | |
| 288 | EVQLQQSGAELVKPGASVKMSCKASVNIFTGYNMHWVKQTPGQGLEWIGGLHPG | 9E7 VH |
| | NGDTSYNQKFKDKATLTADKSSNTAYMQLSSLTSEDSAVYYCALTTAGRAWFAYWG | |
| | QGTLVTVSA | |
| 289 | DIQMTQTTSSLSASLGDRVTISCRASQDISNYLNWYQQKPDGTVKLLIYYTSRLHSGV | 9E7 VL |
| | PSRFSGSGSGTDYSLTISNLEQEDIATYFCQQGNTLPWTFGGGTKLELK | |
| 290 | EVKLVESGGGLVKPGGSLKLSCAASGFTFSRYAMSWVRQTPEKRLEWVATISSGGSY | 24G10 VH |
| | TNYADSVKGRFTISRDNVKNTLYLQMNSLRAEDTAVYYCARATARATEFAYWGQGT | |
| | LVTVSS | |
| 291 | DIQMTQTTSSLSASLGDRVTISCRASQDISNYLNWYQQKPDGTVKLLIY | 24G10 VL |
| | YTSRLHSGVPSRFSGSGSGTDYSLTISNLEQEDIATYFCQQGNTLPPTFGGGTKLEL | |
| 292 | EVQLQQSGAELVKPGASVKMSCKASVNIFTGYNMHWVKQTPGQGLEWIGGLHPG | 25D6 VH |
| | NGDTSYNQKFKDKATLTADKSSNTAYMQLSSLTSEDSAVYYCALTTAGRAWFAYWG | |
| | QGTLVTVSA | |
| 293 | DIQMTQSTSSLSASLGDRVTISCRASQDISNYLNWYQQKPDGTVKLLIYYTSRLHSGV | 25D6 VL |
| | PSRFSGSGSGTDYSLTISNLEQEDIATYFCQQGNTLPWTFGGGTKLEIK | |
| 294 | EVQLKESGAELVKPGASVKMSCKASVNIFTGYNMHWVKQTPGQGLEWIGGLHPG | 25G9 VH |
| | NGDTSYNQKFKDKATLTADRSSNTAYMQLSSLTSEDSAVYYCALTTAGRAWFAYWG | |
| | QGTLVTVSA | |
| 295 | DIQMTQSTSSLSASLGDRVTISCRASQDISNYLNWYQQKPDGTVKLLIYYTSRLHSGV | 25G9 VL |
| | PSRFSGSGSGTDYSLTISNLEQEDIATYFCQQGNTLPWTFGGGTKLEIK | |
| 296 | QVQLKQSGAELVKPGASVKMSCTASVNIFTGYNMHWIKQTPGQGLEWIG | 35A7-1 VH |
| | GLHPGNGDTSYNQKFKDKATLTADRSSNTAYMQLSSLTSEDSAVYYCAL | |
| | TTAGRAWFAYWGQGTLVTVSA | |
| 297 | DIQMNQSTSSLSASLGDRVTISCRASQDISNYLNWYQQKPDGTIKLLIYYTSRLYSGVP | 35A7-1 VL |
| | SRFSGSGSGTDYSLTISNLEEEDIATYFCQQGNTIPYTFGGGTKLEIK | |
| 298 | EVQLQQSGAELVKPGASVKMSCKASVNIFTGYNMHWVKQTPGQGLEWIGGLHPG | 31B9 VH |
| | NGDTSYNQKFKDKATLTADRSSNTAYMQLSSLTSEDSAVYYCALTTAGRAWFAYWG | |
| | QGTLVTVSA | |
| 299 | DIQMTQSTSSLSASLGDRVTISCRASQDISNYLNWYQQKPDGTIKLLIYYTSRLHSGVP | 31B9 VL |
| | SRFSGSGSGTDYSLTISNLEEEDIATYFCQQGNTLPWTFGGGTKLEIK | |
| 300 | EVQLQQSGAELVKPGASVKMSCKASVNIFTSYNMHWVKQTPGQGLEWIGGLHPG | 34A2 VH |
| | NGDTSYNQKFKGKATLTADKSSSTAYMQVSMLTSEDSAVYYCVLTTAGRAWFAYW | |
| | GQGTLVTVSA | |
| 301 | DIQMTQSTSSLSASLGDRVTISCRASQDISNYLNWYQQKPDGTIKLLIYYTSRLHSGVP | 34A2 VL |
| | SRFSGSGSGTDYSLTISNLEEEDIATYFCQQGNTLPWTFGGGTKLEIK | |
| 302 | QVQLQESGAELVKPGASVKMSCKASVNTFTGYNMHWVKQTPGQGLEWIGGLHPG | 34H11 VH |
| | NGDTSYNQKFKGKATLTADRSSNTAYMQLSSLTSEDSAVYYCALTTAGRAWFAYWG | |
| | QGTLVTVSA | |
| 303 | DIQMTQTTSSLSASLGDRVTISCRASQDISNYLNWYQQKPDGTVKLLIYYTSRLHSGV | 34H11 VL |
| | PSRFSGSGSGTDYSLTISNLEEEDIATYFCQQGNTLPWTFGGGTKLEIK | |
| 304 | QVQLKQSGAELVKPGASVKMSCKASVNTFTGYNMHWVKQTPGQGLEWIGGLHPG | 35D5 VH |
| | NGDTSYNQKFKGKATLTADKSSNTAYMQLSSLTSEDSAVYYCALTTAGRAWFAYWG | |
| | QGTLVTVSA | |
| 305 | DIQMTQSTSSLSASLGDRVTISCRASQDISNYLNWYQQKPDGTVKLLIYYTSRLHSGV | 35D5 VL |
| | PSRFSGSGSGTDYSLTISNLEEEDIATYFCQQGNTLPWTFGGGTKLEIK | |
| 306 | QVQLKQSGAELVKPGASVKMSCKASVNTFTGYNMHWIKQTPGQGLEWIGGLHPG | 35A7-2 VH |
| | NGDTSYNQKFKGKATLTADKSSNTAYMQLSSLTSEDSAVYYCALTTAGRAWFAYWG | |
| | QGTLVTVSA | |
| 307 | DIQMNQSTSSLSASLGDRVTISCRASQDISNYLNWYQQKPDGTIKLLIYYTSRLYSGVP | 35A7-2 VL |
| | SRFSGSGSGTDYSLTISNLEEEDIATYFCQQGNTLPYTFGGGTKLEIK | |
| 308 | QVQLQQSGAELVKPGASVKMSCKASVNTITGYNMHWVKQTPGQGLEWIGGLHPG | 36F4 VH |
| | NGDTSYNQKFKDKATLTADKSSNTAYMQLSSLTSEDSAVYYCALTTAGRAWFAYWG | |
| | QGTLVTVSA | |
| 309 | DIKMTQSTSSLSASLGDRVTTSCRASQDISNYLNWYQQKPDGTVKLLIYYTSRLHSGV | 36F4 VL |
| | PSRFSGSGSGTDYSLTISNLEEEDIATYFCQQGNTLPPWTFGGGTKLEIK | |
| 310 | EVQLVESGGGLVKPGGSLRLSCAASGFTFSRYAMSWVRQAPGKGLEWVS | VH1021 |
| | TISSGGSYTNYADSVKGRFTISRDNVKNTLYLQMNSLRAEDTAVYYCARATARATEFA | |
| | YWGQGTLVTVSS | |
| 311 | EVQLLESGGGLVQPGGSLRLSCAASGFTFSRYAMSWVRQAPGKGLEWVS | VH1022 |
| | TISSGGSYTNYADSVKGRFTISRDNVKNTLYLQMNSLRAEDTAVYYCARATARATEFA | |
| | YWGQGTLVTVSS | |
| 312 | QVQLVQSGAELKKPGASVKVSCKASGNTFTGYNMHWIQQSPGQGLEWMGGLHP | VH1023 |
| | GNGDSSYNQKFQGRVTLTADKSSNTAYMELSSLRSEDSAVYYCALTTAGRAWFPYW | |
| | GQGTLVTVSS | |
| 313 | QVQLVQSGAEVKKPGASVKMSCKASVYTFTGYNMHWIQQSPGQGLEWMGGLHP | VH1024 |
| | GNGDSSYNQKFQGRATLTADKSSNTAYMELSSLRSEDSAVYYCALTTAGRAWFPYW | |
| | GQGTLVTVSS | |
| 314 | QVQLVQSGAEVKKPGASVKVSCKASGNIFTGYNMHWVRQAPGQGLEWMGGLHP | VH1025 |
| | GNGDSSYNQKFQGRVTLTADKSSNTAYMELSSLRSEDSAVYYCALTTAGRAWFAYW | |
| | GQGTLVTVSS | |
| 315 | QVQLVQSGAEVKKPGASVKMSCKASVYTFTGYNMHWVRQAPGQGLEWIGGLHP | VH1026 |
| | GNGDSSYNQKFQGRATLTADKSSNTAYMELSSLRSEDSAVYYCALTTAGRAWFAYW | |
| | GQGTLVTVSS | |
| 316 | EVQLVQSGAEVKKPGATVKISCKASGNIFTSYNMHWVRQAPGQGLEWMGGLHPG | VH1027 |
| | NGDTSYNQKFQGRVTLTADKSSSTAYMELSSLRSEDTAVYYCVLTTAGRAWFAYWG | |
| | QGTLVTVSS | |
| 317 | EVQLVQSGAEVKKPGATVKMSCKASVYTFTSYNMHWVRQAPGQGLEWIGGLHPG | VH1028 |
| | NGDTSYNQKFKGRATLTADKSSSTAYMELSSLRSEDTAVYYCVLTTAGRAWFAYWG | |
| | QGTLVTVSS | |
| 318 | DIQMTQSPSSLSASVGDRVTITCQASQDISNYLNWYQQKPGKAPKLLIYYTSRLHSGV | VL1011 |
| | PSRFSGSGSGTDYTFTISSLQPEDIATYFCQQGNTLPWTFGGGTKVEIK | |
| 319 | DIQMTQSPSSLSASVGDRVTITCRASQDISNYLNWYQQKPGKAPKLLIYYTSRLHSGV | VL1012 |
| | PSRFSGSGSGTDYTLTISSLQPEDFATYFCQQGNTLPWTFGGGTKVEIK | |
| 320 | DIQMTQSPSSLSASVGDRVTITCQASQDISNYLNWYQQKPGKAPKLLIYYTSRLHSGV | VL1013 |
| | PSRFSGSGSGTDYTLTISSLQPEDFATYFCQQGNTLPLTFGGGTKVEIK | |
| 321 | DIQMTQSPSSLSASVGDRVTITCQASQDISNYLNWYQQKPGKAPKLLIYYTSRLHSGV | VL1014 |
| | PSRFSGSGSGTDYTLTISSLQPEDFATYFCQQGNTLPWTFGGGTKVEIK | |
| 322 | EVQLQQSGPELVKPGASVKISCKTSGYTFTEYTIHWVKQNHGKSLEWIGGINPNNGG | 13E5 VH |
| | TVYNQNFKGKATLTVDKSSSTAYMELRSLTSEDSAVYYCARVRRGMDYWGQGTSVT | |
| | VSS | |
| 323 | DIVMTQSHKFMSTSVGDRVSITCKASQDVTTAVAWYQQKPGQSPKLLIYSASYRYTG | 13E5 VL |
| | VPDRFTGSGSGTDFTFTISSVQAEDLAVYYCQQHYSAPWTFGGGTNLEIK | |
| 324 | QVQLQQSGAEVVKPGASVKISCKTSGYTFTEYTIHWVKQAHGQSLEWIGGINPNNG | 13E5 H1 VH |
| | GTVYNQKFQGKATLTVDKSTSTAYMELRSLTSEDTAVYYCARVRRGMDYWGQGTS | |
| | VTVSS | |
| 325 | DIQMTQSPKSLSTSVGDRVTITCRASQDVTTAVAWYQQKPGKSPKLLIYSASYRYTGV | 13E5 L1 VL |
| | PSRFSGSGSGTDFTFTISSVQPEDLATYYCQQHYSAPWTFGGGTNLEIK | |
| 326 | QVQLVQSGAEVVKPGASVKVSCKTSGYTFTEYTIHWVRQAPGQSLEWIGGINPNNG | 13E5 H4 VH |
| | GTVYNQKFQGKVTLTVDKSTSTAYMELSSLRSEDTAVYYCARVRRGMDYWGQGTS | |
| | VTVSS | |
| 327 | DIQMTQSPSSLSASVGDRVTITCRASQDVTTAVAWYQQKPGKAPKLLIYSASYRYTG | 13E5 L4 VL |
| | VPSRFSGSGSGTDFTLTISSVQPEDLATYYCQQHYSAPWTFGGGTNLEIK | |
| 328 | EVQLVESGGGLVQPGGSLRLSCAASGIDLRTNSMSWVRQAPGKGLEWVGIIGSSGY | PD2-31 VH |
| | MDYASWAKGRFTISRDNSKNTLYLQMNSLRAEDTAVYYCARHGDSSSFALWGQGT | |
| | LVTVSS | |
| 329 | DIQMTQSPSSLSASVGDRVTITCRASESVYKNNRLSWYQQKPGKAPKLLIYEASKVAS | PD2-31 VL |
| | GVPSRFSGSGSGTDFTLTISSLQPEDFATYYCAGAYRGNIYPFGQGTKVEIK | |
| 330 | EVQLVESGGGLVQPGGSLRLSCAASGFSLNSNAVGWVRQAPGKGLEYIGFINFSGIT | HZD82-12 VH |
| | YYANWAKGRFTISRDNSKNTLYLQMNSLRAEDTAVYYCARGSAGSFDLWGQGTLVT | |
| | VSS | |
| 331 | DIQMTQSPSSLSASVGDRVTITCQASESVYKNNYLAWYQQKPGKAPKLLIYRASNLAS | HZD82-12 VL |
| | GVPSRFSGSGSGTDFTLTISSLQPEDFATYYCQGGYSSGMYPFGQGTKVEIK | |
| 332 | EVQLVESGGGLVKPGGSLKLSCAASGFTFSDYGMHWVRQAPEKGLEWVAFISSGSSI | 25G7 VH |
| | IYYADIVKGRSTISRDNAKNTLFLQMTSLRSEDTAMYYCTRGNKRGFFDYWGQGTILT | |
| | VSS | |
| 333 | QIVLTQSPALMSASPGEKVTMTCNASSSVSYMYWYQRKPRSSPKPWIYLTSNLASG | 25G7 VL |
| | VPVRFSGSGSGTSYSLTISSMEAEDAATYYCQQWRSNPPMLTFGSGTKLEVK | |
| 334 | EVQLVESGGGLVQPGGSLRLSCAASGFTFSDYGMHWVRQAPGKGLEWVAFISSGSS | hu25G7-VH |
| | IIYYADIVKGRSTISRDNAKNTLYLQMNSLRAEDTAVYYCTRGNKRGFFDYWGQGTL | |
| | VTVSS | |
| 335 | EIVLTQSPATLSLSPGERATLSCRASSSVPYMYWYQQKPGQAPRLLIYLTSNLASGIPA | hu25G7-A VL |
| | RFSGSGSGTDFTLTISSLEPEDFAVYYCQQWRAYPPMLTFGGGTKVEIK | |
| 336 | EIVLTQSPATLSLSPGERATLSCRASPGVPPLAWYQQKPGQAPRLLIYLASSRPSGIPA | hu25G7-B VL |
| | RFSGSGSGTDFTLTISSLEPEDFAVYYCQQWRSNPPMLTFGGGTKVEIK | |
| 337 | EVQLVESGGGLVKPGGSLKLSCAASGFTFSDYGMHWVRQAPEKGLEWVAYISSGST | ab 136 VH |
| | TIYYADTVKGRFTISRDNGKNTLFLQMTSLRSEDTAMYYCARISTVVAKRYAMD | |
| | YWGQGTSVTVSS | |
| 338 | DIQMTQTASSLSASLGDRVTISCRASQDISNYLNWYQQKPDGTVKLLIYYTSRLHSGV | ab 136 VL |
| | PSRFSGSGSGTDYSLTISNLEQEDIATYFCQQINALPLTFGAGTKLELK | |
| 339 | EVQLVESGGGLVQPGGSLRLSCAASGFTFSDYGMHWVRQAPGKGLEWVSYISSGST | 136-Hu VH |
| | TIYYADTVKGRFTISRDNAKNSLYLQMNSLRAEDTAVYYCARISTVVAKRYAMDYWG | |
| | QGTLVTVSS | |
| 340 | DIQMTQSPSSLSASVGDRVTITCRASQDISNYLNWYQQKPGKAPKLLIYYTSRLHSGV | 136-Hu VL |
| | PSRFSGSGSGTDFTLTISSLQPEDIATYYCQQINALPFTFGQGTKLEIK | |
| 341 | EVQLVESGGGLVQPGGSLRLSCAVSGFTFSSYAMSWVRQAPGKGLEWVSSITGGG | S1E6 VH |
| | GGIYYADSVKGRFTISRDNSKNTVYLQMNSLRAEDTAVYYCAKDRISITIRPRYFGLDF | |
| | WGQGTTVTVSS | |
| 342 | DIVMTQSPLSLPVTPGEPASISCRSSQSLLYSIGYNYLDWYLQKSGQSPQLLIYLGSNR | L18D7 VL |
| | ASGVPDRFSGSGSGTDFTLKISRVEAEDVGFYYCMQSFKAPYTFGQGTKLEIK | |
| 343 | DIVMTQSPLSLPVTPGEPASISCRSSRNVIYGNGYNYLDWYLQKSGQSPQLLIYLGNN | L28G5 VL |
| | VAAGVPDRFSGSGSGTDFTLKISRVEAEDVGFYYCMQSLQAPYTFGQGTKLEIK | |
| 344 | DIVMTQSPLSLPVTPGEPASISCRSSQNVVYGNGYNYLDWYLQKSGQSPQLLIYLGTN | L28F8 VL |
| | VAAGVPDRFSGSGSGTDFTLKISRVEAEDVGFYYCMQSLQAPYTFGQGTKLEIK | |
| 345 | DIVMTQSPLSLPVTPGEPASISCRSSQNVVYGNGYNYLDWYLQKSGQSPQLLIYLGN | L28C9 VL |
| | NVAAGVPDRFSGSGSGTDFTLKISRVEAEDVGFYYCMQSLKAPYTFGQGTKLEIK | |
| 346 | DIVMTQSPLSLPVTPGEPASISCRSSHNLLYSNGYNYLDWYLQKPGQSPQLLIYLGSN | L10B2 VL |
| | RAYGVPDRFSGSGSGTDFTLKISRVEAEDVGVYHCMQALQSPYTFGQGTKVDIK | |
| 347 | DIVMTQSPLSLPVTAGAPASISCRSSQSLLYSNGYNYLDWYMQKPGQSPQLLIYLGSN | L10C2 VL |
| | RASGVPDRFSGSGSGTDFTLKISRVEAEDVGVYYCMQALETPYAFGQGTKVEIK | |
| 348 | QVQLQQSGPELVKPGASVKISCTASGSTLTDDYINWVKQRPGRGLEWVGWIFPGN | 4-2 VH |
| | GNSYYNEKFKDKATLIVDKSSSTAYMLLSSLTSEDSAVYFCARGLVRYRALFDYWGQG | |
| | TTLTVSS | |
| 349 | QIVLSQSPAILSASPGEKVTMTCRASSSINYMHWYQQKPGSSPKPWIYAASNLASGV | 4-2 VL |
| | PARFSGSGSGTSFSLTISRVEAEDAATYYCQQWSSYPITFGSGTKLEIK | |
| 350 | QVQLVQSGAEVKKPGASVKVSCKASGSTLTDDYINWVRQAPGQRLEWVGWIFPGN | 4-2-Humanized VH |
| | GNSYYNEKFKDRATLTVDKSASTAYMELSSLRSEDTAVYFCARGLVRYRALFDYWGQ | |
| | GTLVTVSS | |
| 351 | DIQMTQSPSSLSASVGDRVTITCRASSSINYMHWYQQKPGKAPKPWIYAASNLASG | 4-2-Humanized VL |
| | VPSRFSGSGSGTDFTLTISSLQPEDFATYYCQQWSSYPITFGQGTKVEIK | |
| 352 | QVQLVESGGGVVQPGRSLRLSCAASGFTFSSYAMHWVRQAPGKGLEWVAVISYDG | BA167 VH |
| | SNKYYADSVKGRFTISRDNSKNTLYLQMNSLRAEDTAVYYCARGLTTVRGVLYWGQ | |
| | GTLVTVSS | |
| 353 | EIVMTQSPSSLSASLGDRVTITCRASQNIGSRLAWYQQKPGKAPKLLIYKASSLESGVP | BA167 VL |
| | SRFSGSGSGTEFTLTISSLQPDDFATYYCQQYNSYSWTFGQGTKLEIK | |
| 354 | EVQLVESGGGLVQPGGSLRLSCAASGFTFSTYGMSWVRQAPGKGLVXVXTINSNGG | huC2C1A1A1 VH |
| | STSYPDSVKGRFTISRDNAKNTLYLQMNSLRAEDTAVYYCARFFRFRNAMDYWGQG | |
| | TLVTVSS | |
| 355 | DIVMTQTPLSLSVTPGQPASISCRSSQSIVHSNGNTYLEWYLQKPGQSPQLLIYKVTN | huB8G11F2B7G5E8 VL |
| | RFSGVPDRFSGSGSGTDFTLKISRVEAEDVGVYYCFQGSHVPYTFGQGTKLEIK | |
| 356 | EVQLVQSGAEVKKPGATVKISCKVSGFNIKDTYMHWVQQAPGKGLEWMGLIDPTN | huB8G11F2B7G5E8-V11 |
| | GYTIYAEKFQGRVTITADTSTDTAYMELSSLRSEDTAVYYCVSRRPWFAYWGQGTLV | VH |
| | TVSS | |
| 357 | QVQLQQSGAELVKPGASVKLSCTASGFNIKDTYMHWVKQRPEQGLEWVGRIDPTN | huB8G11F2B7G5E8-V14 |
| | GYTIYASKFQGKATITADTSSNTAYMQLSSLTSEDTAVYHCVSRRPWFAYWGQGTTL | VH |
| | TVSS | |
| 358 | EVQLVESGGGLVKPGGSLKLSCAASGFTFSSYAMSWVRQTPEKRLEWVAGIRSGGS | mB8D10G7G6E4 VH |
| | YTYYPDTVKGRFTISRDNARNTLYLQMNSLRSEDTAIYYCARGDKLRPYHFDYWGQG | |
| | TTLTVSS | |
| 359 | DIVMTQSHKFMSTSVGDKVSITCKASQDVTTAVAWYQQKPGQSPKLLIYSASYRYTG | mB8D10G7G6E4 VL |
| | VPDRFAGSGSGTDFTVTISTVQAEDLAVYYCQQHYSDPYTFGGGTKLEIK | |
| 360 | EVQLVESGGGLVKPGGSLKLSCAASGFTFSNYAMSWVRQTPEKRLEWVAGIRSGGS | mB9A7C9A4H5 VH |
| | YTYYPDTVKGRFTISRDNARNTLYLQMSSLRSEDTAIYYCARGDKLRPYHFDYWGQG | |
| | TTLTVSS | |
| 361 | DIVMTQSHKFMSTSVGDRVSITCKASQDVSTAVVWYQQKPGQSPKLLIYSASYRYTG | mB9A7C9A4H5 VL |
| | VPDRFTGSGSGTDFTFTIITVQAEDLAVYYCQQHYSAPYTFGGGTQLEIK | |
| 362 | EVKLVESGGGLVKPGGSLKLSCAASGFTFSSYAMSWVRQTPEKRLEWVASISSGDST | mB9D1D11F8D8 VH |
| | YYLDSVKGRFTISRDNARNILYLQVSSLRSEDTAMYYCERSGGSAPYWGQGTLVTVSA | |
| 363 | QIVLTQSPAIMSASPGDMVTISCSASSSVNYMYWYQQKPGSSPKPWIYRTSNLASG | mB9D1D11F8D8 VL |
| | VPARFSGSGSGTSYSLTISSMEAEDAATYYCQQYHSFPLTFGAGTKLELK | |
| 364 | EVKLVESGGGLVKPGGSLKLSCAASGFTFSSYAMSWVRQTPEKRLEWVASISSGDST | mB1D2F7D3B5 VH |
| | YYLDSVKGRFTISRDNAMNILYLQMSSLRSEDTAVYYCERSGGSAPYWGQGTLVSVS | |
| | A | |
| 365 | QIVLTQSPAIMSASPGEMVTISCSASSSVSYMYWYQQKPGSSPKPWIYRTSNLASGV | mB1D2F7D3B5 VL |
| | PARFSGSGSGTSYSLTISSMEAEDAATYYCQQYHSFPLTFGAGTKLELK | |
| 366 | EVQLVESGGGLVQPGGSLRLSCAASGFTFSNFWMTWVRQAPGKGLEWVANMNQ | Anti-BCMA HCVR |
| | DGSEKYYVDSVKGRFTISRDNAKSSLYLQMNSLRAEDTAVYYCARDREYCISTSCYDD | |
| | FDYWGQGTLVTVSS | |
| 367 | GFTFSNFW | Anti-BCMA HCDR1 |
| 368 | MNQDGSEK | Anti-BCMA HCDR2 |
| 369 | ARDREYCISTSCYDDFDY | Anti-BCMA HCDR3 |
| 370 | EVQLVESGGGLVQPGRSLRLSCAASGFTFDDYSMHWVRQAPGKGLEWVSGISWNS | Anti-CD3 HCVR |
| | GSKGYADSVKGRFTISRDNAKNSLYLQMNSLRAEDTALYYCAKYGSGYGKFYHYGLD | (REGN5458) |
| | VWGQGTTVTVSS | |
| 371 | GFTFDDYS | Anti-CD3 HCDR1 |
| | | (REGN5458) |
| 372 | ISWNSGSK | Anti-CD3 HCDR2 |
| | | (REGN5458) |
| 373 | AKYGSGYGKFYHYGLDV | Anti-CD3 HCDR3 |
| | | (REGN5458) |
| 374 | EVQLVESGGGLVQPGRSLRLSCAASGFTFDDYSMHWVRQAPGKGLEWVSGISWNS | Anti-CD3 HCVR |
| | GSIGYADSVKGRFTISRDNAKNSLYLQMNSLRAEDTALYYCAKYGSGYGKFYYYGMD | (REGN5459) |
| | VWGQGTTVTVSS | |
| 375 | GFTFDDYS | Anti-CD3 HCDR1 |
| | | (REGN5459) |
| 376 | ISWNSGSI | Anti-CD3 HCDR2 |
| | | (REGN5459) |
| 377 | AKYGSGYGKFYYYGMDV | Anti-CD3 HCDR3 |
| | | (REGN5459) |
| 378 | DIQMTQSPSSLSASVGDRVTITCRASQSISSYLNWYQQKPGKAPKLLIYAASSLQSGV | Anti-BCMA and Anti-CD3 |
| | PSRFSGSGSGTDFTLTISSLQPEDFATYYCQQSYSTPPITFGQGTRLEIK | LCVR |
| 379 | QSISSY | Anti-BCMA and Anti-CD3 |
| | | LCDR1 |
| 380 | AAS | Anti-BCMA and Anti-CD3 |
| | | LCDR2 |
| 381 | QQSYSTPPIT | Anti-BCMA and Anti-CD3 |
| | | LCDR3 |
| 382 | EVQLVESGGGLVQPGGSLRLSCAASGFTFSNFWMTWVRQAPGKGLEWVANMNQ | REGN5458 heavy chain |
| | DGSEKYYVDSVKGRFTISRDNAKSSLYLQMNSLRAEDTAVYYCARDREYCISTSCYDD | (BCMA-binding arm) |
| | FDYWGQGTLVTVSSASTKGPSVFPLAPCSRSTSESTAALGCLVKDYFPEPVTVSWNS | |
| | GALTSGVHTFPAVLQSSGLYSLSSVVTVPSSSLGTKTYTCNVDHKPSNTKVDKRVESKY | |
| | GPPCPPCPAPPVAGPSVFLFPPKPKDTLMISRTPEVTCVVVDVSQEDPEVQFNWYV | |
| | DGVEVHNAKTKPREEQFNSTYRVVSVLTVLHQDWLNGKEYKCKVSNKGLPSSIEKTIS | |
| | KAKGQPREPQVYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQKSLSLSLGK | |
| 383 | EVQLVESGGGLVQPGRSLRLSCAASGFTFDDYSMHWVRQAPGKGLEWVSGISWNS | REGN5458 heavy chain |
| | GSKGYADSVKGRFTISRDNAKNSLYLQMNSLRAEDTALYYCAKYGSGYGKFYHYGLD | (CD3-binding arm) |
| | VWGQGTTVTVSSASTKGPSVFPLAPCSRSTSESTAALGCLVKDYFPEPVTVSWNSGA | |
| | LTSGVHTFPAVLQSSGLYSLSSVVTVPSSSLGTKTYTCNVDHKPSNTKVDKRVESKYGP | |
| | PCPPCPAPPVAGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNRFTQKSLSLSPGK | |
| 384 | EVQLVESGGGLVQPGGSLRLSCAASGFTFSNFWMTWVRQAPGKGLEWVANMNQ | REGN5459 heavy chain |
| | DGSEKYYVDSVKGRFTISRDNAKSSLYLQMNSLRAEDTAVYYCARDREYCISTSCYDD | (BCMA-binding arm) |
| | FDYWGQGTLVTVSSASTKGPSVFPLAPCSRSTSESTAALGCLVKDYFPEPVTVSWNS | |
| | GALTSGVHTFPAVLQSSGLYSLSSVVTVPSSSLGTKTYTCNVDHKPSNTKVDKRVESKY | |
| | GPPCPPCPAPPVAGPSVFLFPPKPKDTLMISRTPEVTCVVVDVSQEDPEVQFNWYV | |
| | DGVEVHNAKTKPREEQFNSTYRVVSVLTVLHQDWLNGKEYKCKVSNKGLPSSIEKTIS | |
| | KAKGQPREPQVYTLPPSQEEMTKNQVSLTCLVKGFYPSDIAVEWESNGQPENNYKT | |
| | TPPVLDSDGSFFLYSRLTVDKSRWQEGNVFSCSVMHEALHNHYTQKSLSLSLGK | |
| 385 | EVQLVESGGGLVQPGRSLRLSCAASGFTFDDYSMHWVRQAPGKGLEWVSGISWNS | REGN5459 heavy chain |
| | GSIGYADSVKGRFTISRDNAKNSLYLQMNSLRAEDTALYYCAKYGSGYGKFYYYGMD | (CD3-binding arm) |
| | VWGQGTTVTVSSASTKGPSVFPLAPCSRSTSESTAALGCLVKDYFPEPVTVSWNSGA | |
| | LTSGVHTFPAVLQSSGLYSLSSVVTVPSSSLGTKTYTCNVDHKPSNTKVDKRVESKYGP | |
| | PCPPCPAPPVAGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNRFTQKSLSLSPGK | |
| 386 | DIQMTQSPSSLSASVGDRVTITCRASQSISSYLNWYQQKPGKAPKLLIYAASSLQSGV | REGN5458 and |
| | PSRFSGSGSGTDFTLTISSLQPEDFATYYCQQSYSTPPITFGQGTRLEIKRTVAAPSVF | REGN5459 light chain |
| | IFPPSDEQLKSGTASVVCLLNNFYPREAKVQWKVDNALQSGNSQESVTEQDSKDSTYS | |
| | LSSTLTLSKADYEKHKVYACEVTHQGLSSPVTKSFNRGEC | |
| 387 | EVQLVESGGGLEQPGGSLRLSCAGSGFTFRDYAMTWVRQAPGKGLEWVSSISGSG | Dupilumab heavy chain |
| | GNTYYADSVKGRFTISRDNSKNTLYLQMNSLRAEDTAVYYCAKDRLSITIRPRYYGLD | amino acid sequence |
| | VWGQGTTVTVSSASTKGPSVFPLAPCSRSTSESTAALGCLVKDYFPEPVTVSWNSGA | without C-terminal |
| | LTSGVHTFPAVLQSSGLYSLSSVVTVPSSSLGTKTYTCNVDHKPSNTKVDKRVESKYGP | lysine |
| | PCPPCPAPEFLGGPSVFLFPPKPKDTLMISRTPEVTCVVVDVSQEDPEVQFNWYVDG | |
| | VEVHNAKTKPREEQFNSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSLG | |

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Clinical Trial to Assess the Safety, Tolerability, and Pharmacodynamic Effects of Short-Term Linvoseltamab Treatment and Chronic Dupilumab Treatment in Patients with Food Allergy

This example describes a Phase 1 dose-escalation study in adults with severe IgE-mediated food allergy, to assess the safety, tolerability, and pharmacodynamic effects of short-term treatment with linvoseltamab (a BCMAxCD3 bispecific antibody) in combination with chronic treatment with dupilumab (an IL-4R antibody). Without being bound to a particular theory, it is hypothesized that short-term linvoseltamab treatment induces T-cell killing of IgE-producing plasma cells, while chronic dupilumab treatment prevents the formation of new IgE-producing plasma cells.

### Study Design and Objectives

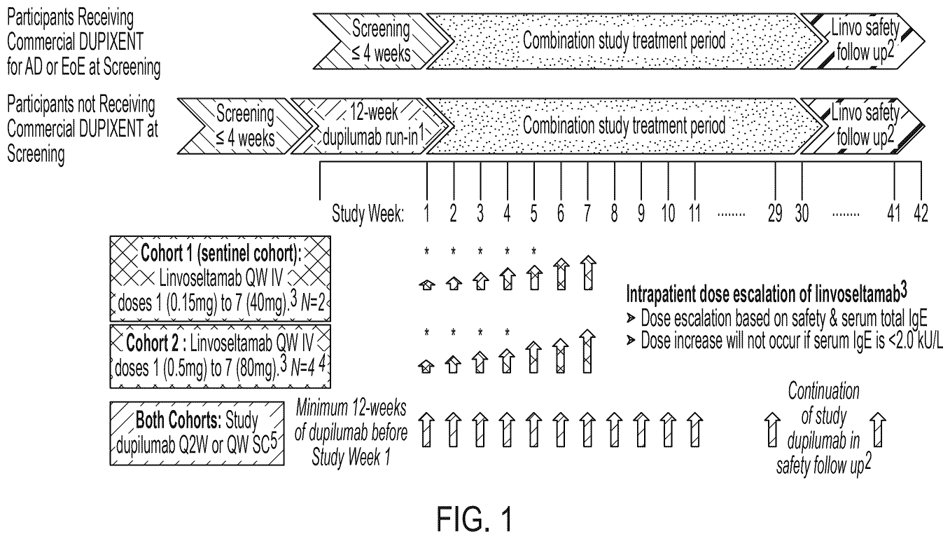

This is a phase 1, open-label, single-arm, multicenter study in participants who have documented severe IgE-mediated food allergy. The study design is shown in FIG. 1. The goal of this study is to demonstrate the safety and tolerability of linvoseltamab in adults with IgE-mediated severe food allergy who receive concomitant dupilumab. This study will evaluate increasing intra-participant dose levels of linvoseltamab to determine the dose that is acceptably safe and tolerable and provides a reduction in total IgE concentrations to below the lower limit of quantification.

The study consists of the following periods:

Screening (up to 4 weeks) and enrollment: Participants must meet eligibility criteria to be enrolled in the study. Two participants will be enrolled into the sentinel cohort (Cohort 1) first. Enrollment for Cohort 2 will commence if the sentinel participants are assessed to tolerate the linvoseltamab doses. Dosing of linvoseltamab will be staggered to allow for evaluation of safety.

Dupilumab run-in (minimum of 12 weeks); Participants not receiving DUPIXENT (dupilumab) prior to screening will enter a 12-week minimum run-in period of dupilumab treatment 300 mg SC Q2W (600 mg SC loading dose) before entering the combination study treatment period. Participants who are receiving DUPIXENT as standard of care for AD (300 mg SC Q2W) or EoE (300 mg SC QW) are not required to complete the 12-week minimum run-in period.

Combination study treatment period (30 weeks): Participants will continue dupilumab treatment as follows: 300 mg SC Q2W (AD or dupilumab naïve participants) or 300 mg SC QW (EoE participants) during the combination study treatment period. In addition, patients may receive up to 7 doses of linvoseltamab. Linvoseltamab will be administered IV at baseline (week 1) and at weeks 2, 3, 4, 5, 6 and 7. In Cohort 1 (sentinel cohort), linvoseltamab will be intra-participant dose escalated to a maximum of 40 mg (starting dose 0.15 mg). In Cohort 2, linvoseltamab will be intra-participant dose escalated to a maximum of 80 mg (starting dose 0.5 mg). After each dose of linvoseltamab, the decision to dose escalate for each participant will be made following review of all available safety and serum total IgE data. Serum total IgE levels will be measured a week before each linvoseltamab dosing day. Linvoseltamab dosing will be stopped if the most recent available serum total IgE concentrations are below the LLOQ (<2.0 kU/L).

Linvoseltamab safety follow-up period: All participants will have a 52-week follow-up period for linvoseltamab safety monitoring following the combination study treatment period. All participants will remain on study dupilumab during this 52-week linvoseltamab safety follow-up period.

At the end of the study, participants who received commercial DUPIXENT prior to enrollment will seamlessly revert to DUPIXENT, as prescribed prior to study enrollment. Participants who were initiated on dupilumab as part of this study may seamlessly continue dupilumab therapy at the end of this study by enrolling in an OLE study. The decision to continue dupilumab treatment under an OLE study will be based on the investigator's clinical judgment and participant discretion. There should be no gap in dupilumab therapy between study treatment and commercial DUPIXENT/dupilumab under this approach, to prevent rebound of IgE levels.

Optional linvoseltamab re-dosing: Participants who have completed the initial 30-week combination study treatment period with linvoseltamab (even if a participant did not receive all 7 doses of linvoseltamab), may be eligible for optional re-dosing of linvoseltamab. All participants who choose re-dosing with linvoseltamab will have a re-dosing 30-week combination study treatment period followed by a re-dosing 52-week follow-up period for linvoseltamab safety monitoring. All participants will remain on study dupilumab during this 52 week linvoseltamab safety follow-up period (without interruption).

The primary objective of the study is to assess the safety and tolerability of linvoseltamab on a background of dupilumab therapy when administered to participants with severe IgE mediated allergy to ingested food.

The key secondary objectives of the study are: (i) to describe the changes of serum total IgE concentrations after administration of linvoseltamab on a background of dupilumab therapy; and (ii) to describe concentrations of immunoglobulin isotypes and IgG subtype in serum over time after administration of linvoseltamab on a background of dupilumab therapy.

Other secondary objectives of the study are: (i) to describe the number of participants who achieve a serum total IgE <2 kU/L (LLOQ); and (ii) to describe the changes of food allergen-specific serum IgE concentrations after administration of linvoseltamab on a background of dupilumab therapy.

### Study Population

The study population comprises adult participants with severe IgE-mediated food allergy, with a history of anaphylaxis requiring epinephrine use, emergency room visit(s), and/or hospitalization for a food-induced reaction.

The following are inclusion criteria for the trial:
(1) Age 18 to 50 years (inclusive) at screening
(2) Clinical history of documented, ongoing, severe IgE-mediated allergy to food (peanut, hazelnut, walnut, cashew, milk, egg/egg white, soy, wheat, sesame, cod, salmon, tuna, lobster, crab and/or shrimp; documented symptom[s] of anaphylaxis due to exposure)
(3) History of physician reported anaphylaxis to food requiring epinephrine administration and/or requiring an emergency visit or inpatient hospitalization
(4) Participants with dupilumab-indicated AD must be receiving DUPIXENT 300 mg SC Q2W as standard of care for the treatment of AD for a minimum of 12 weeks prior to screening, OR participants with dupilumab-indicated EoE must be receiving DUPIXENT 300 mg SC QW as standard of care for the treatment of EoE for a minimum of 12 weeks prior to screening, OR must be willing to initiate dupilumab 300 mg SC Q2W treatment for food allergy
(5) Participants initiating dupilumab treatment must agree to remain on dupilumab 300 mg SC Q2W for the duration of the study treatment period. Participants on commercial DUPIXENT must agree to remain on their prescribed dose (300 mg SC Q2W for AD or 300 mg SC QW for EoE) for the duration of the study treatment period.
(6) Serum total IgE of >150 kU/L during the screening period or prior to start of run-in period
(7) Food-specific IgE to at least one of the following food allergens: peanut, hazelnut, walnut, cashew, milk, egg/egg white, soy, wheat, sesame, cod, salmon, tuna, lobster, crab, shrimp, of ≥0.35 kU/L (serum) during screening period or prior to start of run-in period. Note: Food-specific IgE must be ≥0.35 kU/L for the same allergen to which the participant demonstrated clinical history. Food-specific IgE may also be ≥0.35 kU/L for additional allergens.
(8) Positive SPT to at least one of the following food allergens: peanut, hazelnut, black walnut, English walnut, cashew, milk, egg/egg white, soy, wheat, sesame, cod, salmon, tuna, lobster, crab, shrimp, defined as a mean wheal diameter ≥4 mm wheal compared to saline negative control during the screening period or prior to start of run-in period. Note: SPT must be positive for the same allergen to which the participant demonstrated clinical history. SPT may also be positive for additional allergens.
(9) Participant must be willing to use an epinephrine auto-injector device
(10) Participant must be willing and able to comply with all clinic visits and study-related procedures
(11) Participant must be willing to receive booster and/or re-vaccination(s), including for live (attenuated) vaccinations, based on results of vaccine antibody titers and investigator opinion
(12) Provide informed consent signed by the study participant
(13) Has a body mass index between 18 and 32 kg/m², inclusive
(14) Is judged by the investigator to be in good health based on medical history, physical examination, vital sign measurements, and ECGs performed at screening and/or prior to administration of initial dose of study drug
(15) Is in good health based on laboratory safety testing obtained at the screening visit
(16) Has normal blood pressure readings, defined as normal semi-recumbent blood pressure readings at screening and baseline visits (systolic blood pressure ≤140 and ≥90; diastolic blood pressure ≤90 and ≥50; and resting pulse ≤105 and ≥40.

The following are exclusion criteria for the trial:
(1) Women of childbearing potential (WOCBP) who are unwilling to practice highly effective contraception prior to the initial dose of linvoseltamab for participants who have already been on DUPIXENT for AD or EoE for 12 weeks prior to screening or prior to the start of the dupilumab run-in treatment for participants who are not on DUPIXENT treatment for AD or EoE prior to screening, during the study, and for at least 6 months after the last dose of linvoseltamab (regardless if the participant remained in the study or not) and until end of study whichever is longer.
(2) Sexually active male participants with partners of childbearing potential who are unwilling to use the following forms of medically acceptable birth control at start of the first treatment, during the study, and for at least 6 months after the last dose of linvoseltamab (regardless of if the participant remains in the study or not): vasectomy with medical assessment of surgical success OR consistent use of a condom.
(3) Pregnant or breastfeeding women
(4) History of chronic disease (other than AD or EoE) requiring therapy (e.g., heart disease, diabetes, hypertension) that, in the opinion of the principal investigator, would represent a risk to the participant's health or safety in this study or the participant's ability to comply with the study protocol. Participants on DUPIXENT for conditions other than AD or EoE (e.g., asthma, chronic rhinosinusitis with nasal polyps, prurigo nodularis, etc.) are excluded.
(5) Known or suspected progressive multifocal leukoencephalopathy (PML), or history of PML, neurodegenerative condition, CNS movement disorder, or seizure within 12 months prior to Day 1
(6) Recent history (within past 30 days) of a grade 3 or grade 4 gastrointestinal bleed, history of inflammatory bowel disease or severe diverticulitis or previous gastrointestinal perforation
(7) History of moderate or severe asthma based on the Global Initiative for Asthma (GINA) guidelines
(8) Pre-bronchodilator FEV1<80% of predicted using local reference values
(9) Any history of heart failure or any evidence of symptomatic atherosclerotic cardiovascular disease, such as history of angina, stenting, any vascular bypass procedure, transient ischemic attacks
(10) Cardiac ejection fraction <40% by echocardiogram
(11) Hemoglobin ≤8.0 g/dL
(12) Total bilirubin ≥1.5×ULN
(13) Transaminase (ALT and/or AST) ≥2.5×ULN
(14) Hypogammaglobulinemia, defined as serum total IgG <400 mg/dl during screening
(15) Absolute neutrophil count <1,500 cells/mm³ during screening
(16) Lymphocyte count <1,500 cells/mm³ during screening
(17) CD4 T cell count <400 cells/mm³ during screening
(18) Has an estimated glomerular filtration rate (eGFR) of <60 mL/min/1.73 m² at the screening visit
(19) Any prior exposure to a BCMA targeted therapy
(20) Use of systemic corticosteroids within 2 months prior to screening
(21) Use of omalizumab within 6 months prior to screening
(22) Use of beta-blockers during the screening period or plan to initiate beta-blockers during the treatment period
(23) Use of other forms of allergen immunotherapy (e.g., oral, SC, patch, or sublingual) or immunomodulatory therapy (not including corticosteroids) within 6 months prior to screening
(24) Unwilling to discontinue use of antihistamines within 5 days prior to screening and within 5 days prior to SPT
(25) Known allergy or hypersensitivity to components of linvoseltamab
(26) Known allergy or hypersensitivity to components of dupilumab
(27) Hypersensitivity to epinephrine and any of the excipients in the epinephrine product
(28) Intolerance or allergy to any of the medications used for premedication (dexamethasone, acetaminophen, diphenhydramine)
(29) Contraindication or history of intolerance to IVIG
(30) History of a mast cell disorder, including mastocytosis, urticarial pigmentosa, and hereditary or idiopathic angioedema
(31) Negative for antibodies against Varicella, measles, mumps and/or rubella at screening; for these participants, if they are willing to be vaccinated, they can be rescreened after at least 3 months after vaccination
(32) Treatment with a live (attenuated) vaccine within 3 months prior to screening and during the study
(33) Any active infection within the last 2 weeks prior to baseline
(34) Within the previous 2 months of the screening visit has a history of bacterial, protozoal, viral or parasite infection requiring hospitalization or treatment with IV anti-infectives
(35) Any history of tuberculosis (including active tuberculosis, latent tuberculosis infection (LTBI) or history of incompletely treated tuberculosis or LTBI)
(36) Known history of HIV infection or HIV seropositivity at the screening visit
(37) With an established diagnosis of hepatitis B viral infection at the time of screening or is positive for hepatitis B surface antigen (HBsAg) or hepatitis B core antibody (HBcAb) at the time of screening
(38) Is positive for hepatitis C antibody and positive for qualitative (i.e., detected or not detected) HCV RNA test at the screening visit
(39) Presence of CMV viremia at screening
(40) Has a history of systemic fungal diseases
(41) History of malignancy within 5 years before the screening visit, except completely treated in situ carcinoma of the cervix, completely treated, and resolved non-metastatic squamous or basal cell carcinoma of the skin
(42) Established diagnosis of a primary immunodeficiency disorder (e.g., Severe Combined Immunodeficiency, Wiskott-Aldrich Syndrome, DiGeorge Syndrome, X-linked Agammaglobulinemia, Common Variable Immunodeficiency), or secondary immunodeficiency
(43) Presents any concern to the study investigator that might confound the results of the study or poses an additional risk to the participant by their participation in the study
(44) Was hospitalized (i.e., >24 hours) for any reason within 30 days of the screening visit
(45) Is a current smoker or former smoker, including e-cigarettes, who stopped smoking within 3 months prior to the screening visit
(46) Participated in a clinical study of an investigational biologic drug within 90 days or at least 5 half-lives (whichever is longer), or of other investigational drug within 4 weeks, prior to the screening visit
(47) Immune effector cell-associated encephalopathy (ICE) score less than 10 at screening or baseline
(48) Is receiving a prohibited medication that the participant is unwilling or unable to discontinue (e.g., for medical reasons) during the study

### Study Treatment

The investigational treatments to be administered in the study are linvoseltamab and dupilumab. Table 2 below provides further details about the study interventions to be administered.

TABLE 2
Study Interventions Administered

| | | |
|---|---|---|
| Intervention Label | | |
| Intervention Name | Linvoseltamab | Dupilumab |
| Intervention Description | Cohort 1 (sentinel): 0.15 to 40 | For participants not receiving |
| | mg IV QW for a maximum of 7 | commercial DUPIXENT at baseline: |
| | doses | 300 mg SC Q2W (600 mg loading |
| | Cohort 2: 0.5 to 80 mg IV QW | dose |
| | for a maximum of 7 doses | For participants receiving |
| | | commercial DUPIXENT for AD at |
| | | baseline: 300 mg SC Q2W |
| | | For participants receiving |
| | | commercial DUPIXENT for EoE at |
| | | baseline: 300 mg QW |
| Dose Formulation | Solution for injection | Prefilled syringe |
| Unit Dose Strength(s) | 10 mg/mL | 150 mg/mL |
| Dosage Level(s) | Cohort 1 (sentinel): 0.15 to 40 | 300 mg (600 mg loading dose for |
| | mg | participants not receiving |
| | Cohort 2: 0.5 to 80 mg | commercial DUPIXENT at screening) |
| Route of Administration | Infusion | Subcutaneous injection |
| Use | Experimental | Experimental |

Linvoseltamab drug product is supplied as a liquid solution in sterile, single-use vials for IV administration. Each vial contains linvoseltamab at a concentration of 10 mg/mL (10 mL vial; 5 mL extractable volume). Linvoseltamab dose levels are shown in Table 3 (for the Sentinel Cohort 1) and Table 4 (for Cohort 2).

TABLE 3
Intra-Participant Linvoseltamab Dose
Levels¹ for Cohort 1 (Sentinel Cohort)

| | | | Pre-Medication | |
|---|---|---|---|---|
| | Study | | | Antihistamine |
| | Week | | | (oral or IV) and |
| Dose | (visit | Dose | Dexamethasone | acetaminophen/ |
| number | window) | Level | (20 mg IV) | paracetamol (oral) |
| 1 | Week 1 | 0.15 mg | X | X |
| | (day 1) | IV | | |
| 2 | Week 2 (+3 | 0.5 mg IV | X | X |
| | days) | | | |
| 3 | Week 3 (+3 | 1.5 mg IV | X | X |
| | days) | | | |
| 4 | Week 4 (+3 | 5 mg IV | | X |
| | days) | | | |
| 5 | Week 5 (+3 | 10 mg IV | | X |
| | days) | | | |
| 6 | Week 6 (+3 | 20 mg IV | | X |
| | days) | | | |
| 7 | Week 7 (+3 | 40 mg IV | | X |
| | days) | | | |

¹Dose levels are nominal maximum doses. The actual dose administered may be reduced based on observed safety, tolerability, and/or PK. Doses of linvoseltamab may be modified (lowered) with each new participant dosing based on emerging safety data and available PD and PK data.

TABLE 4
Intra-Participant Linvoseltamab Dose Levels¹ for Cohort 2

| | | | Pre-medication | |
|---|---|---|---|---|
| | Study | | | Antihistamine |
| | Week | | | (oral or IV) and |
| Dose | (visit | Dose | Dexamethasone | acetaminophen/ |
| number | window) | level | (20 mg IV) | paracetamol (oral) |
| 1 | Week 1 (day 1) | 0.5 mg IV | X | X |
| 2 | Week 2 (+3 | 1.5 mg IV | X | X |
| | days) | | | |
| 3 | Week 3 (+3 | 5 mg IV | | X |
| | days) | | | |
| 4 | Week 4 (+3 | 10 mg IV | | X |
| | days) | | | |
| 5 | Week 5 (+3 | 20 mg IV | | X |
| | days) | | | |
| 6 | Week 6 (+3 | 40 mg IV | | X |
| | days) | | | |
| 7 | Week 7 (+3 | 80 mg IV | | X |
| | days) | | | |

¹Dose levels are nominal maximum doses. The actual dose administered may be reduced based on observed safety, tolerability, and/or PK. Doses of linvoseltamab may be modified (lowered) with each new participant dosing based on emerging safety data and available PD and PK data.

Dupilumab drug product is supplied in a pre-filled syringe at a concentration of 150 mg/mL for subcutaneous administration. Each 2.25 mL single-use prefilled syringe with rigid needle shield delivers 300 mg of dupilumab (2.0 mL of a 150 mg/mL solution). Dupilumab injections can be administered by trained study personnel at the site or at home (by participant, caregiver, or healthcare professional). Participants and/or caregivers will be trained on administration of dupilumab and provided with written instructions for home administration. Participants with AD who are already receiving dupilumab will be switched to clinical study supplied dupilumab on their first treatment visit.

Run-in Treatment: For participants not receiving dupilumab for AD or EoE prior to screening, dupilumab 300 mg SC Q2W will be administered during a 12-week run-in. Participants will receive a 600 mg SC loading dose.

Pretreatments: Premedication will be administered prior to each infusion with linvoseltamab to limit the potential for CRS. Premedication will consist of dexamethasone or equivalent (20 mg), antihistamine (diphenhydramine [H1-blocker]) or equivalent (25 mg), and acetaminophen (650 mg) or paracetamol (500 mg).

Permitted Medications: The use of epinephrine is permitted; all participants will be trained on the proper use of epinephrine autoinjector devices and must have access to these at all times. Antihistamines are permitted except within 5 days prior to screening or skin prick tests.

### Study Variables

For this study, baseline characteristics include standard demography (e.g., age, race, weight, height, etc.), medical history, food allergy history and history of symptoms of anaphylaxis due to exposure to allergen, and medication (including dupilumab) history. Safety variables include AEs; vital signs including heart rate, blood pressure, respiration rate, pulse oximetry, weight and body temperature; physical examination findings; ECG results; FEV1 and peak flow; results of laboratory tests including hematology, blood chemistry (including HS CRP, ferritin, and amylase), urinalysis, and pregnancy test or FSH. Pharmacokinetic variables include concentrations of total linvoseltamab, functional dupilumab, and total sBCMA in serum for individual participants and time. Pharmacodynamic variables include serum immunoglobulin (lg) isotype and subtype (including total IgE, food-allergen specific IgEs, total IgA, total IgM, total IgG, IgG1, IgG2 and IgG3) test results and time point/visit.

Endpoints: The primary, key secondary, and secondary endpoints of this study are provided below.

Primary endpoint: Incidence and severity of TEAEs, including AESIs and SAEs, during the combination study treatment period (from first dose of linvoseltamab through the end of the combination study treatment period [i.e., week 30])

Key secondary endpoints: (1) Absolute and percent change from baseline in the serum concentration of total IgE over time to week 30. (2) Time to reach baseline level and/or the lower limit of the normal ranges of serum IgG, IgM and IgA from numerically lowest level, through week 30.

Secondary endpoints: (1) Incidence of participants with unquantifiable concentrations (<2 kU/L) of serum total IgE through week 30. (2) Absolute and percent change from baseline in the serum concentration of food allergen-specific IgE through to week 30 (in participants who tested positive for a measured food allergen-specific IgE at baseline). (3) Time to reach unquantifiable food allergen-specific serum IgE levels (reduction to below LLOQ) through to week 30 (in participants who tested positive for a measured food allergen-specific IgE at baseline).

Immunoglobulin Quantification: Serum samples will be collected as specified time points for quantification of immunoglobulins IgE, IgG, IgA and IgM. These measures are relevant to the mechanism of action of linvoseltamab/dupilumab. Serum samples will be collected as specified time points for food allergen-specific IgEs and IgG quantification (peanut, hazelnut, walnut, cashew, milk, egg/egg white, soy, wheat, sesame, cod, salmon, tuna, lobster, crab, and shrimp). Serum samples will be used to quantify total IgG, IgG1, IgG2 and IgG3. Inhaled/environmental specific IgEs will also be measured (cat, dog, house dust mite, Timothy grass, White Oak, Silver Birch, and ragweed). These measures are relevant for the pathophysiology of food allergy as well as mechanism of action of linvoseltamab/dupilumab.

Skin Prick Test: The standard SPT is performed on the volar surface of the participant's forearm using standardized extract reagents (peanut, hazelnut, black walnut, English walnut, cashew, milk, egg/egg white, soy, wheat, sesame, cod, salmon, tuna, lobster, crab, and shrimp). At screening, a broad panel of reagents will be used; in subsequent tests, only those reagents which yielded a positive result at screening will be used. A positive result is ≥4 mm 15 minutes after applying the lancet. The positive control is histamine base, 6 mg/mL and with a wheal ≥4 mm indicating a valid test. The negative control is glycerol saline.

The SPT is the skin testing for atopic response at different concentrations of allergen extract with saline as negative control and histamine as positive controls. The test will be performed at specified time points. The longest diameter and longest orthogonal diameter will be collected. Mean wheal size (diameter) induced by allergen extract, histamine, and saline at each concentration will be calculated by adding the longest diameter to the longest orthogonal diameter and dividing by 2. Normalized SPT will be calculated by subtracting mean saline wheal size from mean allergen wheal size.

Blood B Cell and T Cell Counts: Peripheral blood will be collected for B cell and T cell counts. Samples will be tested locally using a panel including but not limited to the following markers: CD45, CD3, CD4, CD8, and CD19.

Open Oral Food Challenge: An optional open food challenge may be performed, under intensive monitoring, at the end of the combination study treatment period in participants who (1) have 2 consecutive serum total and food allergen-specific IgE levels to the food below the limit of quantification within 1 week prior to challenge, (2) a negative skin prick test to the food within 2 weeks prior to the challenge (3) a negative skin prick test to the food on the morning of the challenge, and (4) remain on a stable dupilumab dose regimen without interruption during the combination study treatment period. Participants with a peanut, hazelnut, walnut or cashew allergy must demonstrate a negative SPT and have food-allergen specific IgE below the LLOQ for both the whole allergen and any components. Food challenges will be conducted according to the American Academy of Allergy, Asthma and Immunology Work Group Report, using a four-dose protocol (Bird et al., J Allergy Clin Immunol Pract 2020, 8:75-90.e17). The participant's sensitivity to an allergen is defined as the dose at which the participant experiences allergic reactions. Up-dosing during the food challenge will be stopped according to the criteria specified in Bird et al, incorporated by reference herein. After the last dose in the food challenge, the participant will be monitored for at least 2 hours and then discharged from the clinic. Participants will be considered to have tolerated the food challenge and passed if they do not experience any symptoms and/or signs that indicate an allergic reaction. If the participant experiences reactions, they will be treated with the necessary rescue medications.

### Results

### Study Participant #1

Study participant #1 was a 20-year-old male with mild asthma, allergic rhinitis, atopic dermatitis, and multiple severe IgE-mediated food allergies with documented recurrent anaphylaxis (including a clinical history of anaphylaxis to wheat, peanut, and cow's milk), ER visits, and hospitalizations, which significantly impacted his quality of life. Baseline screening data for study participant #1 is shown in Table 5 below. After screening, the participant was administered dupilumab for a 12-week run-in period (600 mg loading dose followed by 300 mg Q2W for subsequent doses). After the 12-week run-in period, the participant was dosed with linvoseltamab as shown in Table 3 for a total of 4 doses of linvoseltamab, each dose administered 7 days apart; for doses 1-3, the participant received pre-medication with dexamethasone 20 mg IV. During the linvoseltamab dosing period, dupilumab treatment Q2W was maintained. As shown in the schematic in FIG. 1, dupilumab treatment Q2W was continued after the last dose of linvoseltamab was administered.

TABLE 5
Baseline Data for Study Participant #1

| Food specific-IgE (kU/L) | Environmental/Perennial IgE (kU/L) | |
|---|---|---|
| (ref range <0.35 kU/L) | (ref range <0.35 kU/L) | Immunoglobulins |
| Peanut: 208.00* | Ragweed: 3.92 | Total IgE: 2215 kU/L |
| | | (ref range 0-119 kU/L) |
| Cashew nut: 61.30 | Silver birch: 21.00 | IgG: 1,012 mg/dL |
| | | (ref range 700-1600 mg/dL) |
| Hazelnut: 64.40 | White oak: 5.76 | IgM: 43 mg/dL |
| | | (ref range 40-230 mg/dL) |
| Walnut: 8.63 | Timothy grass: 4.46 | IgA: 82 mg/dL |
| | | (ref range 70-400 mg/dL) |
| Cow's milk: 138.0* | D farina: 65.30 | |
| Egg white: 21.80 | Dog dander: 26.70 | |
| Wheat: 291.00* | Cat dander: 18.90 | |
| Whole egg: 31.50 | House dust mite: 25.60 | |
| Sesame seed: 48.10 | | |
| Shrimp: 30.90 | | |
| Lobster: 15.60 | | |
| Crab: 20.40 | | |
| Cod: 0.14 | | |
| Salmon: 0.32 | | |
| Soybean: 43.70 | | |
| Tuna: 0.22 | | |

*Recent anaphylaxis/ER visit or IgE >200 kU/L

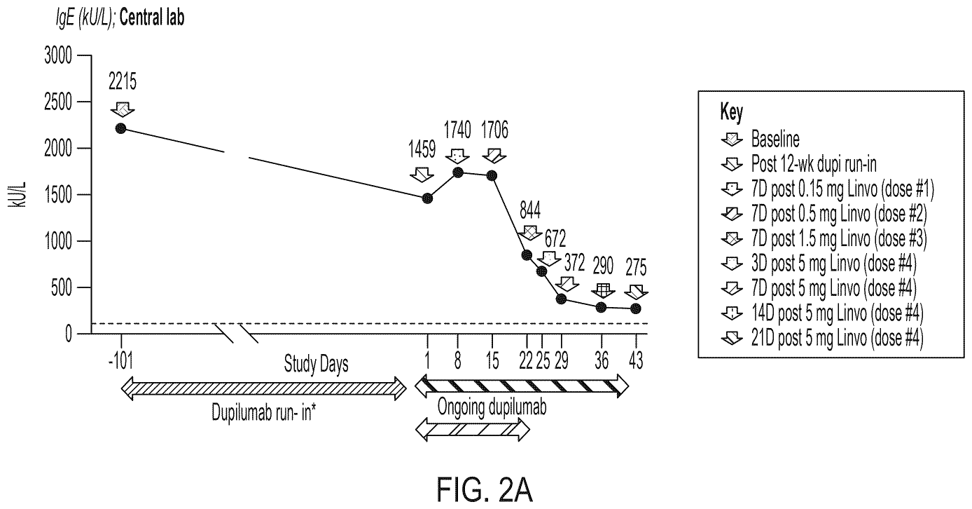

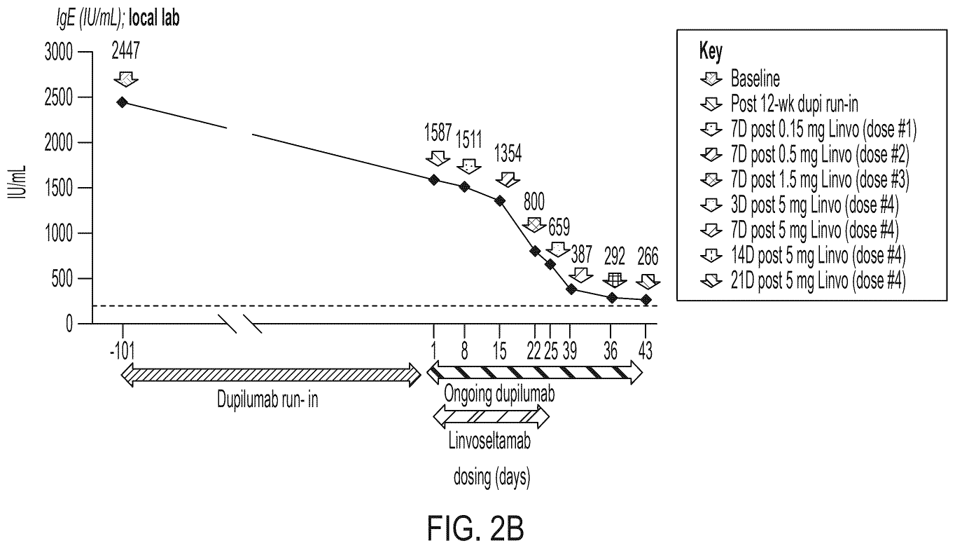

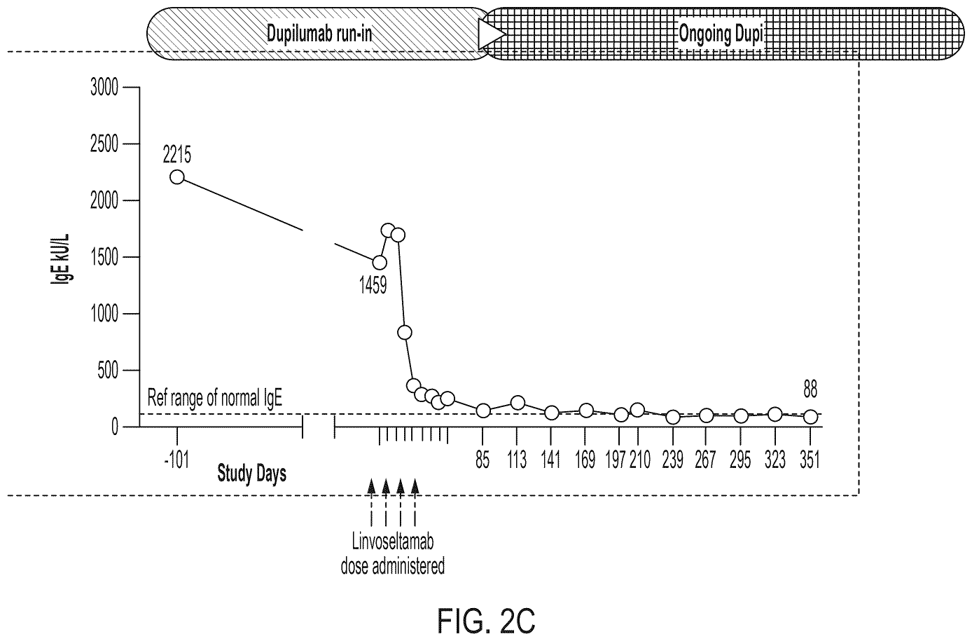

The participant's total IgE levels were measured over time by a central lab and a local lab. As shown in Table 6 below and in FIGS. 2A-2B, total IgE decreased after administration of the initial linvoseltamab doses (up to 5 mg). The decrease in total IgE was sustained at day 43 (6 weeks after the first dose of linvoseltamab was administered, and 3 weeks after the 4th and final dose of linvoseltamab was administered), suggesting that combining linvoseltamab and dupilumab has the potential to eliminate IgE, the key driver of allergic reactions, and durably reverse severe allergies. FIG. 2C shows that the significant reduction in IgE levels (approximately 90% reduction) was sustained with ongoing dupilumab treatment up to study day 351.

TABLE 6
Total IgE Levels Over Time for Study Participant #1
Central lab measurements of IgE (kU/L) levels over time

| | Study Day | Linvo Dose (mg) | | | Reference range |
|---|---|---|---|---|---|
| Visit | Post Linvo | (Dose #) | | IgE (kU/L) | (kU/L) |
| V1 | | N/A | (baseline) | 2215 | 0.11-119.00 |
| V2* | 1 | 0.15 | (1) | 1459 | |
| V3 | 8 | 0.5 | (2) | 1740 | |
| V4 | 15 | 1.5 | (3) | 1706 | |
| V5 | 22 | 5 | (4) | 844 | |

| | | | |
|---|---|---|---|
| V5 + 3 days | 25 | N/A | 672 |
| V6 | 29 | N/A | 372 |
| V7 | 36 | N/A | 290 |
| V8 | 43 | N/A | 275 |

Local lab measurements of IgE (IU/mL) levels over time

| | Study Day | | | Reference range |
|---|---|---|---|---|
| Visit | Post Linvo | Linvo Dose (mg) | IgE (IU/mL) | (IU/mL) |
| V1 | | N/A (baseline) | 2447 | 0-200 |
| V2* | 1 | 0.15 | 1587 | |
| V3 | 8 | 0.5 | 1511 | |
| V4 | 15 | 1.5 | 1354 | |
| V5 | 22 | 5 | 800 | |
| V5 + 3 days | 25 | N/A | 659 | |
| V6 | 29 | N/A | 387 | |
| V7 | 36 | N/A | 292 | |
| V8 | 43 | N/A | 266 | |

Visits (V) are 7 days apart (unless specified otherwise)
*V2 was post 12-week dupilumab run-in; ongoing treatment with dupilumab Q2W kU/L = IU/mL

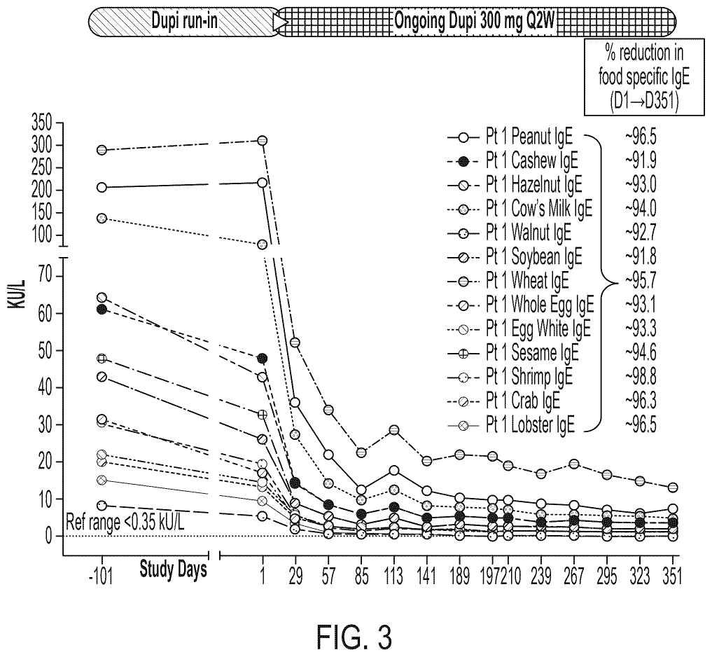

The participant's allergen-specific IgE levels were measured over time by a central lab. As shown in Table 7 below, allergen-specific IgE levels for all food and perennial allergens tested were significantly decreased at Visit 6 (one week after the final dose of linvoseltamab (5 mg) was administered), suggesting that a combination treatment with dupilumab and linvoseltamab has the potential to allow for the introduction of highly allergenic foods into the diets of individuals with severe IgE-mediated allergies. As shown in FIG. 3, for participant #1 there was a greater than 90% reduction in food-specific IgE (peanut, cashew, hazelnut, cow's milk, walnut, soybean, wheat, whole egg, sesame, shrimp, crab, and lobster) from study day 1 to study day 351.

TABLE 7
Allergen-Specific IgE Levels Over Time for Study Participant #1
Allergen-Specific IgE (kU/L) levels over time - Peanut Allergen

| | Linvo | | | | | | |
|---|---|---|---|---|---|---|---|
| | Dose | | | | | | |
| Visit | (mg) | | Peanut | Peanut | Peanut | Peanut | Peanut |
| (wk) | (Dose #) | Peanut | (Ara h1) | (Ara h2) | (Ara h3) | (Ara h6) | (Ara h8) |
| V1 | N/A | 208.00 | 50.10 | 86.20 | 81.60 | 39.80 | 2.75 |
| (screening) | (baseline) | | | | | | |
| V2* (1) | 0.15 (1) | 221.00 | 27.70 | 62.60 | 66.20 | 28.40 | 1.69 |
| V6** (5) | Post 5 mg | 36.40 | 8.02 | 17.00 | 14.80 | 7.67 | 1.15 |
| | (4) | | | | | | |

Allergen-Specific IgE (kU/L) levels over time - Food Allergens

| | Linvo | | | | | | | | |
|---|---|---|---|---|---|---|---|---|---|
| | Dose | | | | | | | | |
| Visit | (mg) | | | | | | Whole | Egg | |
| (wk) | (Dose #) | Cashew | Hazelnut | Walnut | Soybean | Wheat | Egg | White | Sesame |
| V1 | N/A | 61.30 | 64.40 | 8.63 | 43.70 | 291.00 | 31.50 | 21.80 | 48.10 |
| (screening) | (baseline) | | | | | | | | |
| V2* (1) | 0.15 (1) | 48.20 | 42.70 | 5.73 | 26.60 | 312.00 | 17.30 | 15.10 | 33.10 |
| V6** (5) | Post 5 mg | 14.60 | 15.10 | 2.20 | 8.99 | 52.50 | 5.55 | 4.82 | 9.11 |
| | (4) | | | | | | | | |

Allergen-Specific IgE (kU/L) levels over time - Peanut Allergen

| | Linvo Dose | | | | | | | |
|---|---|---|---|---|---|---|---|---|
| | (mg) | | | | | | | |
| Visit | (Dose #) | | | | | | | |
| (wk) | Linvo Dose | Peanut | Peanut | Peanut | Peanut | Peanut | Peanut | |
| Visit | (mg) | Cow's | (Ara h1) | (Ara h2) | (Ara h3) | (Ara h6) | (Ara h8) | |
| (wk) | (Dose #) | Milk | Shrimp | Crab | Cod | Lobster | Salmon | Tuna |
| V1 | N/A | 138.00 | 30.90 | 20.40 | 0.14 | 15.60 | 0.32 | 0.22 |
| (screening) | (baseline) | | | | | | | |
| V2* (1) | 0.15 (1) | 79.80 | 19.60 | 13.60 | <0.10 | 9.73 | 0.19 | 0.14 |
| V6** (5) | Post 5 mg | 27.60 | 6.13 | 4.85 | <0.10 | 3.24 | <0.10 | <0.10 |
| | (4) | | | | | | | |

Allergen-Specific IgE (kU/L) levels over time - Perennial Allergens

| | Linvo | | | | | | | | |
|---|---|---|---|---|---|---|---|---|---|
| | Dose | | | | House | | | | |
| Visit | (mg) | Dog | Cat | D. | Dust | White | Timothy | | Silver |
| (wk) | (Dose #) | Dander | Dander | farina | Mite | Oak | Grass | Ragweed | Birch |
| V1 | N/A | 26.70 | 18.90 | 65.30 | 25.60 | 5.76 | 4.46 | 3.92 | 21.00 |
| (screening) | (baseline) | | | | | | | | |
| V2* (1) | 0.15 (1) | 17.70 | 14.40 | 41.40 | 17.70 | 3.20 | 2.67 | 3.51 | 11.50 |
| V6** (5) | Post 5 mg | 7.80 | 4.20 | 13.10 | 5.36 | 1.02 | 0.97 | 1.73 | 3.56 |
| | (4) | | | | | | | | |

*Post 12-week dupilumab run-in; ongoing treatment with dupilumab Q2W
**Linvoseltamab 5 mg administered during V5
Reference range for all allergens is <0.35 kU/L.

The levels of other serum immunoglobulins for Study Participant #1 were measured over time, including total IgG, total IgA, and total IgM (Table 8). The IgG decrease was in line with expected decrease for linvoseltamab active doses. Notably, by Visit 8 (3 weeks after the final dose of linvoseltamab was administered), IgG levels had not fallen below 400 mg/dL, which per trial protocol was the cutoff value for discontinuing linvoseltamab treatment and initiating IVIG immunoglobulin replacement therapy.

TABLE 8
IgG, IgA, and IgM Levels Over Time for Study Participant #1

| | | Linvo Dose | | | | |
|---|---|---|---|---|---|---|
| | Study Day | (mg) | | IgG (mg/dL) | IgA (mg/dL) | IgM (mg/dL) |
| Visit | Post-Linvo | (Dose #) | | Ref: 700-1600 | Ref: 70-400 | Ref: 40-230 |
| V1 | | N/A | (baseline) | 1012 | 82 | 43 |
| V2 | 1 | 0.15 | (1) | 1110 | 88 | 42 |
| V3 | 8 | 0.5 | (2) | 1011 | 78 | 41 |
| V4 | 15 | 1.5 | (3) | 839 | 53 | 34 |
| V5 | 22 | 5 | (4) | 714 | 31 | 20 |

| | | | | | |
|---|---|---|---|---|---|
| V5 + 3 days | 25 | N/A | 629 | 24 | 17 |
| V6 | 29 | N/A | 611 | 18 | 14 |
| V7 | 36 | N/A | 552 | 13 | 11 |
| V8 | 43 | N/A | 553 | 11 | 8 |

Visits (V) are 7 days apart (unless specified otherwise)
* Post 12-weeks dupilumab run-in; ongoing treatment with dupilumab Q2W

### Study Participant #2

Study participant #2 is a 23-year-old male with EoE (on dupilumab for approximately 2.5 years), mild asthma, allergic rhinitis, and multiple severe IgE-mediated food allergies with documented recurrent anaphylaxis, ER visits, and hospitalizations. Baseline screening data for study participant #2 is shown in Table 9 below. No dupilumab run-in period was required because the participant was already on dupilumab for EoE (300 mg QW). The participant was intravenously dosed with linvoseltamab as shown in Table 3 for a total of 7 weekly doses of linvoseltamab, up to a dose of 40 mg. The participant received pre-medication with dexamethasone 20 mg IV prior to all 7 doses. During the linvoseltamab dosing period, dupilumab treatment QW was maintained.

TABLE 9
Baseline Data for Study Participant #2

| Food specific-IgE (kU/L) | Environmental/Perennial IgE (kU/L) | |
|---|---|---|
| (ref range <0.35 kU/L) | (ref range <0.35 kU/L) | Immunoglobulins |
| Peanut: 0.26 | Ragweed: <0.10 | Total IgE: 147 kU/L |
| | | (ref range 0-119 kU/L) |
| Cashew nut: 1.72* | Silver birch: 4.15 | IgG: 1,302 mg/dL |
| | | (ref range 700-1600 mg/dL) |
| Hazelnut: 4.44* | White oak: 3.58 | IgM: 126 mg/dL |
| | | (ref range 40-230 mg/dL) |
| Walnut: 8.55* | Timothy grass: <0.10 | IgA: 101 mg/dL |
| | | (ref range 70-400 mg/dL) |
| Cow's milk: 7.33* | D farina: 0.15 | |
| Egg white: <0.10 | Dog dander: 0.5 | |
| Wheat: 0.29 | Cat dander: 0.17 | |
| Whole egg: <0.10 | House dust mite: <0.10 | |
| Sesame: 28.00* | | |
| Shrimp: <0.10 | | |
| Lobster: <0.10 | | |
| Crab: <0.10 | | |
| Cod: 0.11 | | |
| Salmon: 0.11 | | |
| Soybean: 0.13 | | |
| Tuna: <0.10 | | |

*Recent anaphylaxis/ER visit

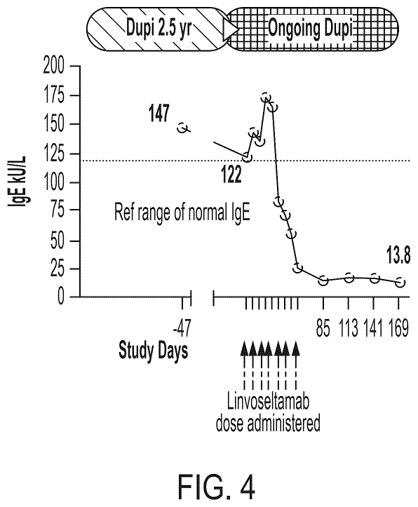

The participant's total IgE levels were measured over time. As shown in FIG. 4, a significant reduction in IgE levels was achieved and was sustained with ongoing dupilumab treatment as of study day 169.

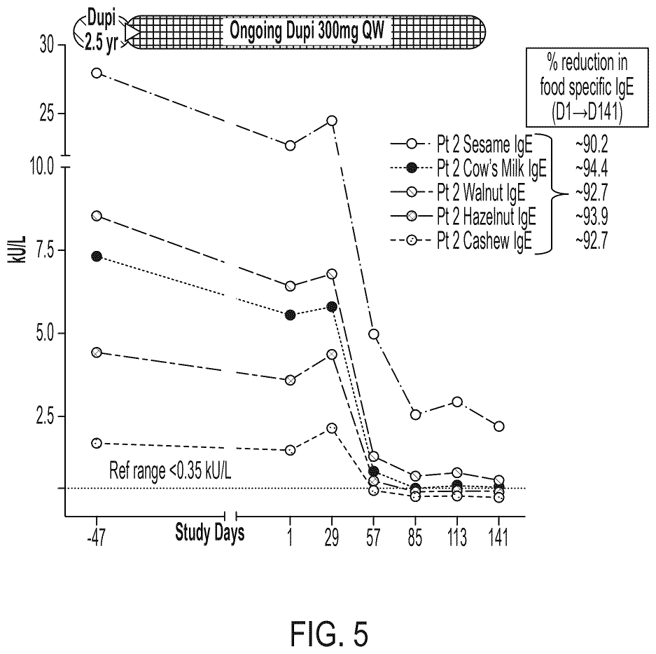

Allergen-specific IgE levels were measured over time. As shown in FIG. 5, for participant #2 there was a greater than 90% reduction in food-specific IgE (sesame, cow's milk, walnut, hazelnut, and cashew) from study day 1 to study day 141.

The total IgG, total IgA, and total IgM levels declined for Study Participant #2 after administration of linvoseltamab (data not shown). As of study day 169, IgG levels had not fallen below 400 mg/dL, which per trial protocol was the cutoff value for discontinuing linvoseltamab treatment and initiating IVIG immunoglobulin replacement therapy.

### Study Participant #3

Study participant #3 is a 27-year-old female with mild asthma, allergic rhinitis, and multiple severe IgE-mediated food allergies (sesame/peanut/tree nuts) with documented recurrent anaphylaxis, ER visits, and hospitalizations. Baseline screening data for study participant #3 is shown in Table 10 below. After screening, the participant was administered dupilumab for approximately 17 weeks (600 mg loading dose followed by 300 mg Q2W for subsequent doses). After the run-in period, the participant was dosed with linvoseltamab as shown in Table 4 for a total of 7 weekly doses of linvoseltamab, up to a dose of 80 mg. The participant received pre-medication with dexamethasone 20 mg IV prior to all 7 doses. During the linvoseltamab dosing period, dupilumab treatment Q2W was maintained.

TABLE 10
Baseline Data for Study Participant #3

| Food specific-IgE (kU/L) | Environmental/Perennial IgE (kU/L) | |
|---|---|---|
| (ref range <0.35 kU/L) | (ref range <0.35 kU/L) | Immunoglobulins |
| Sesame: 223.0* | Ragweed: 0.19 | Total IgE: 499 kU/L |
| | | (ref range 0-119 kU/L) |
| Peanut: 24.80* | Silver birch: 3.16 | IgG: 1,222 mg/dL |
| | | (ref range 700-1600 mg/dL) |
| Cashew nut: 13.50* | White oak: 6.79 | IgM: 83 mg/dL |
| | | (ref range 40-230 mg/dL) |
| Hazelnut: 19.40* | Timothy grass: 7.00 | IgA: 223 mg/dL |
| | | (ref range 70-400 mg/dL) |
| Walnut: 0.89* | D farina: 8.68 | |
| Cow's milk: 0.27 | Dog dander: 34.60 | |
| Egg white: 0.21 | Cat dander: 60.90 | |
| Wheat: 1.31 | House dust mite: 0.55 | |
| Whole egg: 0.16 | | |
| Shrimp: <0.10 | | |
| Lobster: <0.10 | | |
| Crab: <0.10 | | |
| Cod: <0.10 | | |
| Salmon: <0.10 | | |
| Soybean: 12.80 | | |
| Tuna: <0.10 | | |

*Recent anaphylaxis/ER visit

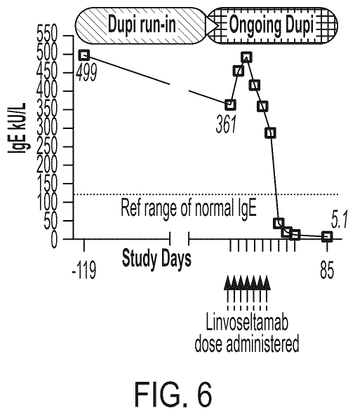

The participant's total IgE levels were measured over time. As shown in FIG. 6, a significant reduction in IgE levels was achieved and was sustained with ongoing dupilumab treatment as of study day 85.

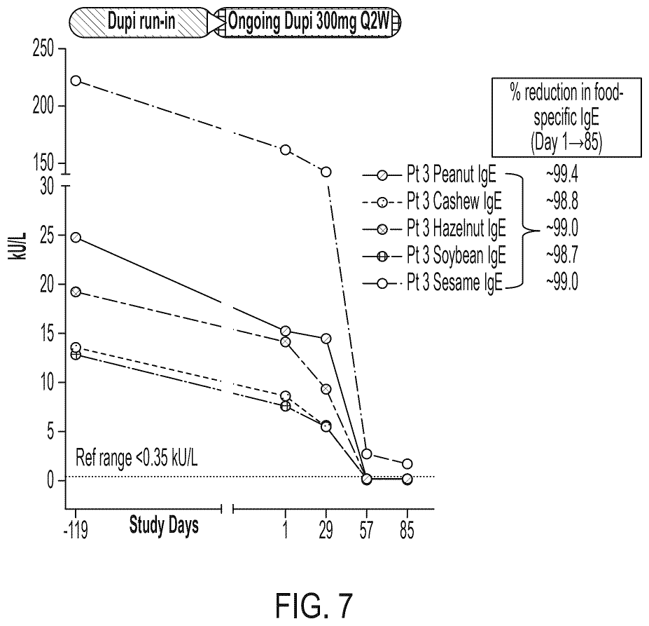

Allergen-specific IgE levels were measured over time. As shown in FIG. 7, for participant #2 there was a greater than 98% reduction in food-specific IgE (peanut, cashew, hazelnut, soybean, and sesame) from study day 1 to study day 85.

The total IgG, total IgA, and total IgM levels declined for Study Participant #3 after administration of linvoseltamab (data not shown). As of study day 85, IgG levels had not fallen below 400 mg/dL, which per trial protocol was the cutoff value for discontinuing linvoseltamab treatment and initiating IVIG immunoglobulin replacement therapy.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method of treating a subject having an IgE-mediated allergy or preventing or reducing the severity of an allergic reaction in a subject having an IgE-mediated allergy, the method comprising:
administering to the subject an interleukin-4 receptor (IL-4R) antagonist, wherein the IL-4R antagonist comprises an antibody that specifically binds IL-4Rα or an antigen-binding fragment thereof; and
administering to the subject a B-cell maturation antigen (BCMA) targeting agent, wherein the BCMA targeting agent comprises a bispecific antigen-binding molecule comprising a first antigen-binding domain that specifically binds BCMA and a second antigen-binding domain that specifically binds CD3;
wherein prior to administering the BCMA targeting agent, the IL-4R antagonist is administered to the subject for a length of time sufficient to attain a steady state concentration of the IL-4R antagonist in the subject.

2. A method for reducing serum total IgE in a subject having an IgE-mediated allergy, the method comprising:
administering to the subject an interleukin-4 receptor (IL-4R) antagonist, wherein the IL-4R antagonist comprises an antibody that specifically binds IL-4Rα or an antigen-binding fragment thereof; and
administering to the subject a B-cell maturation antigen (BCMA) targeting agent, wherein the BCMA targeting agent comprises a bispecific antigen-binding molecule comprising a first antigen-binding domain that specifically binds BCMA and a second antigen-binding domain that specifically binds CD3;
wherein prior to administering the BCMA targeting agent, the IL-4R antagonist is administered to the subject for a length of time sufficient to attain a steady state concentration of the IL-4R antagonist.

3. A method for reducing allergen-specific IgE in a subject having an IgE-mediated allergy, the method comprising:
administering to the subject an interleukin-4 receptor (IL-4R) antagonist, wherein the IL-4R antagonist comprises an antibody that specifically binds IL-4Rα or an antigen-binding fragment thereof; and
administering to the subject a B-cell maturation antigen (BCMA) targeting agent, wherein the BCMA targeting agent comprises a bispecific antigen-binding molecule comprising a first antigen-binding domain that specifically binds BCMA and a second antigen-binding domain that specifically binds CD3;
wherein prior to administering the BCMA targeting agent, the IL-4R antagonist is administered to the subject for a length of time sufficient to attain a steady state concentration of the IL-4R antagonist.

4. The method of any one of claims 1 to 3, wherein the IL-4R antagonist is administered to the subject for at least 12 weeks prior to administering the BCMA targeting agent.

5. The method of any one of claims 1 to 4, wherein the subject has an IgE-mediated food allergy.

6. The method of any one of claims 1 to 5, wherein the subject has an allergy to one or more of peanut, hazelnut, walnut, cashew, milk, egg, egg white, soy, wheat, sesame, cod, salmon, tuna, lobster, crab, and shrimp.

7. The method of any one of claims 1 to 6, wherein the subject has an allergy to multiple foods.

8. The method of any one of claims 1 to 7, wherein the subject is an adult.

9. The method of any one of claims 1 to 8, wherein the subject has a baseline serum total IgE ≥150 kU/L.

10. The method of any one of claims 1 to 9, wherein at least two doses of the BCMA targeting agent are administered to the subject.

11. The method of claim 10, wherein the BCMA targeting agent is administered to the subject once weekly (QW).

12. The method of any one of claims 1 to 11, wherein no more than 7 doses of the BCMA targeting agent are administered to the subject.

13. The method of any one of claims 1 to 9, wherein a single dose of the BCMA targeting agent is administered to the subject.

14. The method of any one of claims 1 to 13, wherein:
(i) the BCMA targeting agent is administered at a dose of from about 0.15 mg to about 80 mg; or
(ii) the BCMA targeting agent is administered at a dose of about 0.5 mg; or
(iii) the BCMA targeting agent is administered at a dose of about 1.5 mg; or
(iv) the BCMA targeting agent is administered at a dose of about 5 mg; or
(v) the BCMA targeting agent is administered at a dose of about 10 mg; or
(vi) the BCMA targeting agent is administered at a dose of about 20 mg; or
(vii) the BCMA targeting agent is administered at a dose of about 40 mg; or
(viii) the BCMA targeting agent is administered at a dose of about 80 mg.

15. The method of any one of claims 1 to 14, wherein the IL-4R antagonist is administered to the subject at a dose of 50 mg to 600 mg.

16. The method of claim 15, wherein the IL-4R antagonist is administered to the subject at a dose of 200 mg or 300 mg.

17. The method of claim 15 or 16, wherein the IL-4R antagonist is administered to the subject once weekly (QW) or once every two weeks (Q2W).

18. The method of claim 15, wherein the IL-4R antagonist is administered to the subject as an initial dose followed by one or more secondary doses, wherein:
the IL-4R antagonist is administered as an initial dose of 400 mg followed by one or more secondary doses of 200 mg; or
the IL-4R antagonist is administered as an initial dose of 600 mg followed by one or more secondary doses of 300 mg.

19. The method of claim 18, wherein each secondary dose is administered one week or two weeks after the immediately preceding dose.

20. The method of any one of claims 1 to 19, wherein the anti-IL-4Rα antibody or antigen-binding fragment thereof comprises three heavy chain complementarity determining regions (HCDR1, HCDR2 and HCDR3) contained within a heavy chain variable region (HCVR) comprising the amino acid sequence of SEQ ID NO:1, and three light chain complementarity determining regions (LCDR1, LCDR2 and LCDR3) contained within a light chain variable region (LCVR) comprising the amino acid sequence of SEQ ID NO:2.

21. The method of any one of claims 1 to 20, wherein the anti-IL-4Rα antibody or antigen-binding fragment thereof comprises an HCDR1 comprising the amino acid sequence of SEQ ID NO:3, an HCDR2 comprising the amino acid sequence of SEQ ID NO:4, an HCDR3 comprising the amino acid sequence of SEQ ID NO:5, an LCDR1 comprising the amino acid sequence of SEQ ID NO:6, an LCDR2 comprising the amino acid sequence of SEQ ID NO:7, and an LCDR3 comprising the amino acid sequence of SEQ ID NO:8.

22. The method of any one of claims 1 to 21, wherein the anti-IL-4Rα antibody or antigen-binding fragment thereof comprises an HCVR comprising the amino acid sequence of SEQ ID NO:1 and an LCVR comprising the amino acid sequence of SEQ ID NO:2.

23. The method of any one of claims 1 to 22, wherein the anti-IL-4Rα antibody is a full antibody.

24. The method of claim 23, wherein the full antibody is an IgG antibody.

25. The method of claim 23 or 24, wherein the full antibody is an IgG4 antibody.

26. The method of any one of claims 1 to 25, wherein the anti-IL-4Rα antibody comprises a heavy chain comprising the amino acid sequence of SEQ ID NO:9 or SEQ ID NO:387 and a light chain comprising the amino acid sequence of SEQ ID NO:10.

27. The method of any one of claims 1 to 26, wherein the IL-4R antagonist is dupilumab.

28. The method of any one of claims 1 to 19, wherein the IL-4R antagonist is AMG317, APG808, BA2101, comekibart, manfidokimab, LQ036, MEDI 9314, NS-402, QX-005N, rademikibart, stapokibart, or TQH-2722.

29. The method of any one of claims 1 to 28, wherein the BCMA targeting agent is an anti-BCMA x anti-CD3 bispecific antibody or antigen-binding fragment thereof comprising a first antigen-binding domain that specifically binds BCMA and a second antigen-binding domain that specifically binds CD3;
wherein the first antigen-binding domain comprises the HCDRs of an HCVR comprising the amino acid sequence of SEQ ID NO:366 and the LCDRs of an LCVR comprising the amino acid sequence of SEQ ID NO:378; and
wherein the second antigen-binding domain comprises the HCDRs of an HCVR comprising the amino acid sequence of SEQ ID NO:370 or SEQ ID NO:374 and the LCDRs of an LCVR comprising the amino acid sequence of SEQ ID NO:378.

30. The method of claim 29, wherein the first antigen-binding domain comprises an HCDR1 comprising the amino acid sequence of SEQ ID NO:367, an HCDR2 comprising the amino acid sequence of SEQ ID NO:368, an HCDR3 comprising the amino acid sequence of SEQ ID NO:369, an LCDR1 comprising the amino acid sequence of SEQ ID NO:379, an LCDR2 comprising the amino acid sequence of SEQ ID NO:380, and an LCDR3 comprising the amino acid sequence of SEQ ID NO:381; and wherein the second antigen-binding domain comprises an HCDR1 comprising the amino acid sequence of SEQ ID NO:371, an HCDR2 comprising the amino acid sequence of SEQ ID NO:372, an HCDR3 comprising the amino acid sequence of SEQ ID NO:373, an LCDR1 comprising the amino acid sequence of SEQ ID NO: 379, an LCDR2 comprising the amino acid sequence of SEQ ID NO:380, and an LCDR3 comprising the amino acid sequence of SEQ ID NO:381.

31. The method of claim 30, wherein the first antigen-binding domain comprises an HCVR comprising the amino acid sequence of SEQ ID NO:366 and an LCVR comprising the amino acid sequence of SEQ ID NO:378; and wherein the second antigen-binding domain comprises an HCVR comprising the amino acid sequence of SEQ ID NO:370 and an LCVR comprising the amino acid sequence of SEQ ID NO:378.

32. The method of claim 30 or 31, wherein the first antigen-binding domain comprises a heavy chain comprising the amino acid sequence of SEQ ID NO:382 and a light chain comprising the amino acid sequence of SEQ ID NO:386; and wherein the second antigen-binding domain comprises a heavy chain comprising the amino acid sequence of SEQ ID NO:383 and a light chain comprising the amino acid sequence of SEQ ID NO:386.

33. The method of any one of claims 30 to 32, wherein the BCMA targeting agent is linvoseltamab.

34. The method of claim 29, wherein the first antigen-binding domain comprises an HCDR1 comprising the amino acid sequence of SEQ ID NO:367, an HCDR2 comprising the amino acid sequence of SEQ ID NO:368, an HCDR3 comprising the amino acid sequence of SEQ ID NO:369, an LCDR1 comprising the amino acid sequence of SEQ ID NO:379, an LCDR2 comprising the amino acid sequence of SEQ ID NO:380, and an LCDR3 comprising the amino acid sequence of SEQ ID NO:381; and wherein the second antigen-binding domain comprises an HCDR1 comprising the amino acid sequence of SEQ ID NO:375, an HCDR2 comprising the amino acid sequence of SEQ ID NO:376, an HCDR3 comprising the amino acid sequence of SEQ ID NO:377, an LCDR1 comprising the amino acid sequence of SEQ ID NO:379, an LCDR2 comprising the amino acid sequence of SEQ ID NO:380, and an LCDR3 comprising the amino acid sequence of SEQ ID NO:381.

35. The method of claim 34, wherein the first antigen-binding domain comprises an HCVR comprising the amino acid sequence of SEQ ID NO:366 and an LCVR comprising the amino acid sequence of SEQ ID NO:378; and wherein the second antigen-binding domain comprises an HCVR comprising the amino acid sequence of SEQ ID NO:374 and an LCVR comprising the amino acid sequence of SEQ ID NO:378.

36. The method of claim 34 or 35, wherein the first antigen-binding domain comprises a heavy chain comprising the amino acid sequence of SEQ ID NO:384 and a light chain comprising the amino acid sequence of SEQ ID NO:386; and wherein the second antigen-binding domain comprises a heavy chain comprising the amino acid sequence of SEQ ID NO:385 and a light chain comprising the amino acid sequence of SEQ ID NO:386.

37. The method of any one of claims 34 to 36, wherein the BCMA targeting agent is vonsetamig.

38. The method of any one of claims 1 to 28, wherein the BCMA targeting agent is alnuctamab, elranatamab, pacanalotamab, pavurutamab, teclistamab, CM336, EM801, EMB-06, HBM7020, TNB-383B, TNB-384B, TQB2934, or YKST02.

39. The method of any one of claims 1 to 38, wherein the level of serum total IgE in the subject is reduced by at least 50%.

40. The method of any one of claims 1 to 39, wherein the level of serum total IgE in the subject is reduced to <2 kU/L.

41. The method of any one of claims 1 to 40, wherein after administering the BCMA targeting agent to the subject, the method further comprises determining the concentration of total IgE in a serum sample from the subject; wherein a further dose of the BCMA targeting agent is administered to the subject only if the subject has a serum total IgE concentration ≥2 KU/L.

42. A method of treating a subject having an IgE-mediated food allergy or preventing or reducing the severity of an allergic reaction in a subject having an IgE-mediated food allergy, the method comprising:
administering to the subject an antibody that specifically binds IL-4Rα, wherein the antibody is dupilumab; and
administering to the subject an anti-BCMA x anti-CD3 bispecific antibody or an antigen-binding fragment thereof;
wherein prior to administering the anti-BCMA x anti-CD3 bispecific antibody or an antigen-binding fragment thereof, the dupilumab is administered to the subject for at least 12 weeks, wherein the dupilumab is administered: (i) at a dosage of 300 mg every week (QW) or every two weeks (Q2W); or (ii) at an initial dose of 600 mg followed by one or more secondary doses of 300 mg, wherein each secondary dose is administered one week or two weeks after the immediately preceding dose.

43. The method of claim 42, wherein the anti-BCMA x anti-CD3 bispecific antibody is alnuctamab, elranatamab, linvoseltamab, pacanalotamab, pavurutamab, teclistamab, vonsetamig, CM336, EM801, EMB-06, HBM7020, TNB-383B, TNB-384B, TQB2934, or YKST02.

44. The method of claim 42 or 43, wherein the anti-BCMA x anti-CD3 bispecific antibody is linvoseltamab.

45. The method of claim 42 or 43, wherein the anti-BCMA x anti-CD3 bispecific antibody is vonsetamig.

46. The method of any one of claims 42 to 45, wherein the subject has an allergy to one or more of peanut, hazelnut, walnut, cashew, milk, egg, egg white, soy, wheat, sesame, cod, salmon, tuna, lobster, crab, and shrimp.

47. The method of any one of claims 42 to 46, wherein the subject:
(i) has an allergy to multiple foods;
(ii) has an allergy to one or more perennial allergens; and/or
(iii) has a baseline serum total IgE ≥150 kU/L.

48. The method of any one of claims 42 to 47, wherein the anti-BCMA x anti-CD3 bispecific antibody is administered at a dose of from about 0.15 mg to about 80 mg.

49. The method of any one of claims 42 to 48, wherein at least two doses of the anti-BCMA x anti-CD3 bispecific antibody are administered to the subject.

50. The method of claim 49, wherein the anti-BCMA x anti-CD3 bispecific antibody is administered to the subject once weekly (QW).

51. The method of any one of claims 42 to 48, wherein a single dose of the anti-BCMA x anti-CD3 bispecific antibody is administered to the subject.
